SEQUENCE LISTING (23 sequences)

SEQ ID NO: 1 - protein, 117 aa, synthetic construct
  source 1..117: note = Synthetic amino acid sequnce of consensus allergen CA1
MASSAAMKLALVVLLCMVVAAPIAEAITCGQVSSSLAPCISYLKSGGAVPPACCNGIRSLNGAAKTTADRQAACNCLKSAAGSISGLNPGLAAGLPGKCGVNIPYKISTSTNCATVK

SEQ ID NO: 2 - protein, 113 aa, synthetic construct
  source 1..113: note = Synthetic amino acid sequnce of consensus allergen CA2
SAVLKLALVVVLCMVVAAPMAAITCGQVTSNLAPCINYLRSGGAVPPACCNGVRNLNNAARTTADRQAACNCLKQAAGSIKGLNPNNAAGLPGKCGVNIPYKISPSTNCATVK

SEQ ID NO: 3 - protein, 116 aa, synthetic construct
  source 1..116: note = Synthetic amino acid sequnce of consensus allergen CA3
MASAMLKLALVVVLCMVVVAAAAAAITCGQVSSSLAPCISYVRGGGAVPPACCNGIRSLNNAARTTADRQAACNCLKQAAGSISGLNPNNAASLPGKCGVNIPYKISTSTNCATVK

SEQ ID NO: 4 - protein, 115 aa, synthetic construct
  source 1..115: note = Synthetic amino acid sequence of consensus allergen CA4
MAAVVLALVVALCMVVAAAASEAAITCGQVSSSLAPCLSYVRGGGAVPSACCSGVRNLNSAAKTTADRQAACNCLKNAAGSISGLNAGNAASLPGKCGVSIPYKISTSTDCSRVK

SEQ ID NO: 5 - DNA, 351 bp, synthetic construct
  source 1..351: mol_type = other DNA
atggccagcagcgccgccatgaagctggccctggtggtgctgctgtgcatggtggtggccgcccccatcgccgaggccatcacctgcggccaggtgagcagcagcctggccccctgcatcagctacctgaagagcggcggcgccgtgccccccgcctgctgcaacggcatcagaagcctgaacggcgccgccaagaccaccgccgacagacaggccgcctgcaactgcctgaagagcgccgccggcagcatcagcggcctgaaccccggcctggccgccggcctgcccggcaagtgcggcgtgaacatcccctacaagatcagcaccagcaccaactgcgccaccgtgaag

SEQ ID NO: 6 - DNA, 351 bp, synthetic construct
  source 1..351: mol_type = other DNA
atggcnwsnwsngcngcnatgaarytngcnytngtngtnytnytntgyatggtngtngcngcnccnathgcngargcnathacntgyggncargtnwsnwsnwsnytngcnccntgyathwsntayytnaarwsnggnggngcngtnccnccngcntgytgyaayggnathmgnwsnytnaayggngcngcnaaracnacngcngaymgncargcngcntgyaaytgyytnaarwsngcngcnggnwsnathwsnggnytnaayccnggnytngcngcnggnytnccnggnaartgyggngtnaayathccntayaarathwsnacnwsnacnaaytgygcnacngtnaar

SEQ ID NO: 7 - DNA, 339 bp, synthetic construct
  source 1..339: mol_type = other DNA
agcgccgtgctgaagctggccctggtggtggtgctgtgcatggtggtggccgcccccatggccgccatcacctgcggccaggtgaccagcaacctggccccctgcatcaactacctgagaagcggcggcgccgtgccccccgcctgctgcaacggcgtgagaaacctgaacaacgccgccagaaccaccgccgacagacaggccgcctgcaactgcctgaagcaggccgccggcagcatcaagggcctgaaccccaacaacgccgccggcctgcccggcaagtgcggcgtgaacatcccctacaagatcagccccagcaccaactgcgccaccgtgaag

SEQ ID NO: 8 - DNA, 339 bp, synthetic construct
  source 1..339: mol_type = other DNA; note = synthetic consensus DNA sequnce encoding CA2
wsngcngtnytnaarytngcnytngtngtngtnytntgyatggtngtngcngcnccnatggcngcnathacntgyggncargtnacnwsnaayytngcnccntgyathaaytayytnmgnwsnggnggngcngtnccnccngcntgytgyaayggngtnmgnaayytnaayaaygcngcnmgnacnacngcngaymgncargcngcntgyaaytgyytnaarcargcngcnggnwsnathaarggnytnaayccnaayaaygcngcnggnytnccnggnaartgyggngtnaayathccntayaarathwsnccnwsnacnaaytgygcnacngtnaar

SEQ ID NO: 9 - DNA, 348 bp, synthetic construct
  source 1..348: mol_type = other DNA; note = synthetic codon optimized DNA sequnce encoding CA3
atggccagcgccatgctgaagctggccctggtggtggtgctgtgcatggtggtggtggccgccgccgccgccgccatcacctgcggccaggtgagcagcagcctggccccctgcatcagctacgtgagaggcggcggcgccgtgccccccgcctgctgcaacggcatcagaagcctgaacaacgccgccagaaccaccgccgacagacaggccgcctgcaactgcctgaagcaggccgccggcagcatcagcggcctgaaccccaacaacgccgccagcctgcccggcaagtgcggcgtgaacatcccctacaagatcagcaccagcaccaactgcgccaccgtgaag

SEQ ID NO: 10 - DNA, 348 bp, synthetic construct
  source 1..348: mol_type = other DNA; note = synthetic consensus DNA sequnce encoding CA3
atggcnwsngcnatgytnaarytngcnytngtngtngtnytntgyatggtngtngtngcngcngcngcngcngcnathacntgyggncargtnwsnwsnwsnytngcnccntgyathwsntaygtnmgnggnggnggngcngtnccnccngcntgytgyaayggnathmgnwsnytnaayaaygcngcnmgnacnacngcngaymgncargcngcntgyaaytgyytnaarcargcngcnggnwsnathwsnggnytnaayccnaayaaygcngcnwsnytnccnggnaartgyggngtnaayathccntayaarathwsnacnwsnacnaaytgygcnacngtnaar

SEQ ID NO: 11 - DNA, 345 bp, synthetic construct
  source 1..345: mol_type = other DNA; note = synthetic codon optimized DNA sequence encoding CA4
atggccgccgtggtgctggccctggtggtggccctgtgcatggtggtggccgccgccgccagcgaggccgccatcacctgcggccaggtgagcagcagcctggccccctgcctgagctacgtgagaggcggcggcgccgtgcccagcgcctgctgcagcggcgtgagaaacctgaacagcgccgccaagaccaccgccgacagacaggccgcctgcaactgcctgaagaacgccgccggcagcatcagcggcctgaacgccggcaacgccgccagcctgcccggcaagtgcggcgtgagcatcccctacaagatcagcaccagcaccgactgcagcagagtgaag

SEQ ID NO: 12 - DNA, 345 bp, synthetic construct
  source 1..345: mol_type = other DNA; note = synthetic consensus DNA sequnce encoding CA4
atggcngcngtngtnytngcnytngtngtngcnytntgyatggtngtngcngcngcngcnwsngargcngcnathacntgyggncargtnwsnwsnwsnytngcnccntgyytnwsntaygtnmgnggnggnggngcngtnccnwsngcntgytgywsnggngtnmgnaayytnaaywsngcngcnaaracnacngcngaymgncargcngcntgyaaytgyytnaaraaygcngcnggnwsnathwsnggnytnaaygcnggnaaygcngcnwsnytnccnggnaartgyggngtnwsnathccntayaarathwsnacnwsnacngaytgywsnmgngtnaar

SEQ ID NO: 13 - RNA, 351 nt, synthetic construct
  source 1..351: mol_type = other RNA; note = synthetic mRNA encoding CA1
atggccagcagcgccgccatgaagctggccctggtggtgctgctgtgcatggtggtggccgcccccatcgccgaggccatcacctgcggccaggtgagcagcagcctggccccctgcatcagctacctgaagagcggcggcgccgtgccccccgcctgctgcaacggcatcagaagcctgaacggcgccgccaagaccaccgccgacagacaggccgcctgcaactgcctgaagagcgccgccggcagcatcagcggcctgaaccccggcctggccgccggcctgcccggcaagtgcggcgtgaacatcccctacaagatcagcaccagcaccaactgcgccaccgtgaag

SEQ ID NO: 14 - RNA, 339 nt, synthetic construct
  source 1..339: mol_type = other RNA; note = synthetic mRNA encoding CA2
agcgccgtgctgaagctggccctggtggtggtgctgtgcatggtggtggccgcccccatggccgccatcacctgcggccaggtgaccagcaacctggccccctgcatcaactacctgagaagcggcggcgccgtgccccccgcctgctgcaacggcgtgagaaacctgaacaacgccgccagaaccaccgccgacagacaggccgcctgcaactgcctgaagcaggccgccggcagcatcaagggcctgaaccccaacaacgccgccggcctgcccggcaagtgcggcgtgaacatcccctacaagatcagccccagcaccaactgcgccaccgtgaag

SEQ ID NO: 15 - RNA, 348 nt, synthetic construct
  source 1..348: mol_type = other RNA; note = synthetic mRNA encoding CA3
atggccagcgccatgctgaagctggccctggtggtggtgctgtgcatggtggtggtggccgccgccgccgccgccatcacctgcggccaggtgagcagcagcctggccccctgcatcagctacgtgagaggcggcggcgccgtgccccccgcctgctgcaacggcatcagaagcctgaacaacgccgccagaaccaccgccgacagacaggccgcctgcaactgcctgaagcaggccgccggcagcatcagcggcctgaaccccaacaacgccgccagcctgcccggcaagtgcggcgtgaacatcccctacaagatcagcaccagcaccaactgcgccaccgtgaag

SEQ ID NO: 16 - RNA, 345 nt, synthetic construct
  source 1..345: mol_type = other RNA; note = synthetic mRNA encoding CA4
atggccgccgtggtgctggccctggtggtggccctgtgcatggtggtggccgccgccgccagcgaggccgccatcacctgcggccaggtgagcagcagcctggccccctgcctgagctacgtgagaggcggcggcgccgtgcccagcgcctgctgcagcggcgtgagaaacctgaacagcgccgccaagaccaccgccgacagacaggccgcctgcaactgcctgaagaacgccgccggcagcatcagcggcctgaacgccggcaacgccgccagcctgcccggcaagtgcggcgtgagcatcccctacaagatcagcaccagcaccgactgcagcagagtgaag

SEQ ID NO: 17 - protein, 91 aa, Prunus persica
ITCGQVSSALAPCIPYVRGGGAVPPACCNGIRNVNNLARTTPDRQAACNCLKQLSASVPGVNPNNAAALPGKCGVHIPYKISASTNCATVK

SEQ ID NO: 18 - protein, 93 aa, Juglans regia
VITCGQVASSVGSCIGYLRGTVPTVPPSCCNGVKSLNKAAATTADRQAACECLKKTSGSIPGLNPGLAAGLPGKCGVSVPYKISTSTNCKAVK

SEQ ID NO: 19 - protein, 91 aa, Malus domestica
ITCGQVTSSLAPCIGYVRSGGAVPPACCNGIRTINGLARTTADRQTACNCLKNLAGSISGVNPNNAAGLPGKCGVNVPYKISTSTNCATVK

SEQ ID NO: 20 - protein, 159 aa, synthetic construct
MGVFNYETETTSVIPAARLFKAFILDGDNLIPKVAPQAISSVENIEGNGGPGTIKKITFPEGSPFKYVKERVDEVDHANFKYSYSVIEGGALGDTLEKISNEIKIVASPDGGSILKISSKYHTKGDHEIKAEQVKAGKEKAEGLFKAVESYLLAHDAYN

SEQ ID NO: 21 - protein, 160 aa, synthetic construct
MGVFTYETEFTSVIPPARLFKAFVLDADNLIPKIAPQAIKSVEIIEGNGGPGTIKKITFGEGSQYKYVKHRVDEIDKDNFKYSYSVIEGDALSDTLEKISYEIKLVASPDGGSIIKSTSKYHTKGDVEIKEEHVKAGKEKASGLFKAVESYLLAHPDAYN

SEQ ID NO: 22 - protein, 150 aa, synthetic construct
FNYETETTSVIPAARLFKAFILDGDNLIPKVAPQAISSVENIEGNGGPGTIKKITFPEGSPFKYVKERVDEVDHANFKYSYSVIEGGPVGDTLEKISNEIKIVATPDGGSILKISNKYHTKGDHEVKAEQIKASKEKGETLLRAVESYLL

SEQ ID NO: 23 - protein, 160 aa, synthetic construct
MGVFTYETEFTSVIPPARLFKAFVLDADNLIPKIAPQAIKSVEIIEGDGGPGTIKKITFGEGSQYKYVKHRVDEIDKDNFTYSYSLIEGDALSDTLEKISYEIKLVASPDGGSIIKSTSKYHTKGDVEIKEEHVKAGKEKASGLFKAIESYLLAHPDAYN

(19) United States
(12) Patent Application Publication
(10) Pub. No.: US 2025/0367280 A1
(43) Pub. Date: Dec. 4, 2025

(54) ALLERGY VACCINES BASED ON CONSENSUS ALLERGENS

(71) Applicant: Danmarks Tekniske Universitet, Kongens Lyngby (DK)

(72) Inventors: Esperanza Rivera de Torre, Kongens Lyngby (DK); Andreas Hougaard Laustsen-Kiel, Kongens Lyngby (DK); Timothy Patrick Jenkins, Kongens Lyngby (DK)

(21) Appl. No.: 18/874,844

(22) PCT Filed: Jun. 19, 2023
(86) PCT No.: PCT/EP2023/066434

§ 371 Date: Dec. 13, 2024

(30) Foreign Application Priority Data
Jun. 17, 2022 (EP) 22179541.2
Apr. 5, 2023 (EP) 23166732.0

(51) Int. Cl. A61K 39/35; A61K 39/00; A61P 37/04; C07K 14/415

(52) U.S. Cl. CPC A61K 39/35; A61P 37/04; C07K 14/415; A61K 2039/545; A61K 2039/6018

(57) ABSTRACT

The present invention relates to synthetic and/or recombinant consensus allergens and to their use as allergy vaccines in particular in the treatment of peach-cypress allergy, in the form of an allergy vaccine comprising a consensus allergen and/or a nucleic acid sequence encoding such, wherein the consensus allergen comprises at least (60) amino acids and is derived from a consensus sequence of the amino acid sequences of at least five (5) protein allergens, and wherein said protein allergens share at least 20% amino acid sequence identity.

### FIELD

The present invention relates to synthetic and/or recombinant consensus allergens and to their use as allergy vaccines, in particular in the treatment of peach-cypress allergy syndrome.

### BACKGROUND

Allergy is a severe reaction of the immune system to foreign substances commonly known as allergens, with respiratory and food allergies affecting as much as 30% of the global population. Furthermore, there is a structural similarity between pollen and food allergens, which manifests itself in many people suffering from both types of allergies at the same time. Notably, such allergies have a significant negative impact on quality of life, social interactions, and economics for the afflicted. The only available treatments for this chronic condition are either focused on reducing the symptomatology (e.g., with antihistamines), or long-term desensitization via allergen-specific immunotherapy (AIT), which consists of 3-5 years of monthly exposure to low doses of the triggering allergen with the aim of desensitizing the patients' immune system against the triggering allergen. Few patients choose to undergo AIT due to the time required, the significant side effects, a low chance of prolonged protection, and that the efficacy is limited to a single allergen at a time, leaving patients vulnerable to other allergens.

Patients with pollen allergies are frequently allergic to a wide variety of substances in pollen but also in food-stuff. This cross-reactivity occurs because pollens contain epitopes that are conserved across multiple species. Westernberg et al., (Journal of Allergy and Clinical Immunology, Vol. 138, issue 2, 2016, pages 571-578.e7) discloses that antigenic proteins are more conserved than nonimmunogenic proteins and that P. pratense epitopes, which are highly conserved epitopes across pollens, elicit a higher T-cell response in donors with food and/or pollen allergy, compared to less conserved epitopes. They therefore propose the potential use of conserved peptides/epitopes in diagnostics or immunomodulatory approaches, addressing multiple pollen allergies. Westernberg et al., disclose epitopes of 10-15 amino acids and investigate 27 different epitopes. They propose that cocktails of peptides could be used in diagnostics or as immunotherapeutic reagents to simultaneously target multiple pollen allergies.

### SUMMARY OF THE INVENTION

The current invention is based on the design of a new class of synthetic and/or recombinant consensus allergens that can be used to desensitize a patients' immune system against multiple allergens at the same time.

Consequently, in a first aspect, the current invention relates to an allergy vaccine comprising a consensus allergen and/or a nucleic acid sequence encoding said consensus allergen, wherein the consensus allergen comprises at least 60 amino acids and is derived from the amino acid sequences of at least five (5) protein allergens, which share at least 20% amino acid sequence identity. The consensus allergen itself being not a wild-type allergen. In some embodiments, the consensus allergen is a polypeptide.

Furthermore, in some cases, the protein allergens from which the consensus allergen is derived share at least 20%, 30%, 40%, 50%, 60%, 70%, or at least 80% amino acid sequence identity over a sequence length of at least 60, 65, 70, 85, 90, 95, 100, 110 or 115 amino acids. Some protein allergens share at least 20% amino acid sequence identity and at least 70%, such as at least 75%, such as at least 80%, such as at least 85%, such as at least 90%, such as at least 95% sequence similarity over a sequence length of at least 60, such as at least 65, such as at least 70, such as at least 85, such as at least 90, such as at least 95, such as at least 100, such as at least 110, or such as at least 115 amino acids. The group of protein allergens, such as wild-type protein allergens may comprise at least 5, 7, such as at least 10, such as at least 20, such as at least 50, such as at least 75, or such as at least 100 allergen sequences.

In some embodiments, the consensus allergen sequence is obtained by sequence alignment of said protein allergens.

In particular, said consensus allergen can be obtained by:
a) selecting at least five (5) amino acid sequences of protein allergens, such as wild-type protein allergens as defined herein,
b) performing an alignment of the amino acid sequences of said protein allergens, and
c) determining the de novo consensus sequence of said protein allergens from said alignment, wherein the selection of amino acids in the consensus sequence is based on the incidence number of the specific amino acid in each specific position (n) in the sequence of said alignment.

In addition, for the selection of conserved amino acids in the consensus sequence, when two or more amino acids are equally represented in a position (n) in the aligned sequences, the amino acid with the highest molecular volume, according to table 3, is selected as the conserved amino acid. In cases where two amino acids remain equally represented in a position (n) following selection of the amino acid with the highest molecular volume, according to table 3, the conserved amino acid is selected based on the physicochemical properties of amino acid according to the following groups:
group 1 [polar] comprising Asn, Gln, Ser, and Thr,
group 2 [aliphatic] comprising Val, Ala, Leu, Ile, and Met,
group 3 [basic] comprising Lys, Arg, and His,
group 4 [acidic] comprising Asp, and Glu,
group 5 [aromatic] comprising Phe, Trp, and Tyr,
group 6 comprising Pro,
group 7 comprising Gly, and
group 8 comprising Cys,
wherein the conserved amino acid in the specific position is selected based on the selection criteria defined in table 2.

In embodiments, the vaccine comprises a polypeptide comprising the consensus allergen.

In additional embodiments, the vaccine comprises more than one consensus allergen, which can be in the form of more than one polypeptide.

In additional embodiments, the vaccine comprises more than one consensus allergens, which can be in the form of more than one polypeptide, wherein the consensus allergen(s) are derived from at least five wild-type protein allergens.

In more embodiments, the vaccine comprises a nucleotide construct encoding the consensus allergen, and, optionally, the nucleic acid construct comprises at least one self-amplifying mRNA, or at least one non-replicating mRNA sequence, in addition, the nucleic acid construct may comprise a 5′-end cap, one or more coding sequence(s) (CDS), a poly-A tail and/or a replicase encoding one or more nucleic acid sequence(s). The nucleic acid construct may further comprise one or more elements selected from the groups consisting of 5′-end UTR, 3′-end UTR, β-globin leader sequence, capO and capI, and one or more modified nucleotides, such as sugar modified nucleotides, backbone modified nucleotides, base modified nucleotides and unnatural bases. Furthermore, the nucleic acid construct in some embodiments comprises at least one further nucleic acid sequence encoding an RNA sequence and/or a polypeptide.

Both the nucleic acid construct and/or the polypeptide may be encapsulated in a nanoparticle, such a nanoparticle comprising one or more elements selected from the group consisting of lipids, proteins, peptides, dendrimers, protamines, polymers, polysaccharide, mixtures thereof and conjugates thereof.

In some embodiments, the vaccine comprises at least one further adjuvant, such as an adjuvant selected from the group consisting of aluminium salt-based adjuvants, emulsion adjuvants, TLR agonists, CpG-DNA and cytokines.

In embodiments, the consensus allergen according to the current invention is derived from the consensus sequence of non-specific Lipid Transfer Proteins (nsLTP). For example the consensus allergen comprises or consists of an amino acid sequence selected from the group consisting of SEQ ID NOs 1-4 and functional homologues thereof with an amino acid sequence, which is at least 80%, 85%, 90%, 95%, 97%, 98%, or 99% identical to any one of SEQ ID NOs 1-4.

In embodiments, the consensus allergen according to the current invention is derived from the consensus sequence of pathogenic-related protein family 10 (PR10). For example, the consensus allergen comprises or consists of an amino acid sequence selected from the group consisting of SEQ ID NOs 20-23 and functional homologues thereof with an amino acid sequence, which is at least 80%, 85%, 90%, 95%, 97%, 98%, or at least 99% identical to any one of SEQ ID NOs 20-23.

A vaccine of the present invention may be adapted for intramuscular, intradermal, intravenous, transdermal, topical, sublingual, subcutaneous, oral, nasal, ophthalmic and/or biolistic administration.

The invention also relates to a vaccine of the present invention for use as a medicament, such as for use in the treatment of allergy, such as for use in the treatment of peach-cypress syndrome.

In particular, the invention relates to a use of a vaccine according to the invention for treating, ameliorating and/or preventing allergy and/or for the manufacture of a protective and/or therapeutic vaccine for hyposensitizing an individual to an allergen.

Another aspect of the invention relates to an isolated DNA or RNA molecule comprising a nucleic acid sequence encoding a consensus allergen with an amino acid sequence selected from the group consisting of SEQ ID NOs 1-4 and functional homologues thereof with an amino acid sequence which is at least 80%, 85%, 90%, 95%, 97%, 98%, or 99% identical to any one of SEQ ID NOs 1-4.

A further aspect of the invention relates to an isolated DNA or RNA molecule comprising a nucleic acid sequence encoding a consensus allergen with an amino acid sequence selected from the group consisting of SEQ ID NOs 20-23 and functional homologues thereof with an amino acid sequence which is at least 80%, 85%, 90%, 95%, 97%, 98%, or 99% identical to any one of SEQ ID NOs 20-23.

Another aspect of the invention relates to an isolated DNA or RNA molecule comprising a nucleic acid sequence encoding a consensus allergen with an amino acid sequence selected from the group consisting of SEQ ID NOs 1, 2, 3, 4, 20, 21, 22 and 23 and functional homologues thereof with an amino acid sequence which is at least 80%, 85%, 90%, 95%, 97%, 98%, or 99% identical to any one of SEQ ID NOs 1, 2, 3, 4, 20, 21, 22, or 23.

Embodiments also relate to a therapeutic composition comprising such an isolated RNA or DNA. In further embodiments, such an isolated DNA or RNA sequences or a composition comprising such an isolated DNA or RNA sequences, is intended for use as a medicament.

Said vaccine may be administered by intramuscular, intradermal, intravenous, transdermal, topical, sublingual, subcutaneous, oral, nasal, ophthalmic, and/or biolistic administration, preferably, by subcutaneous administration.

In embodiments, the vaccine disclosed herein may be administered in an amount in range of 1-1000 ug pr. dose, such as e.g., in the range of 5-50 ug pr. dose when the vaccine is an mRNA vaccine, or in the range of 10-100 ug pr. dose when the vaccine is a protein-based vaccine.

In embodiments, the vaccine disclosed herein is used as a prophylactic treatment. In other embodiments, the vaccine disclosed herein is for use in the treatment of allergy, such as e.g., in the treatment of peach-cypress allergy or birch-apple syndrome. In further embodiments, wherein the vaccine disclosed herein is used to ameliorate allergy symptoms.

Yet another aspect of the invention relates to a method for providing a consensus allergen for use in a vaccine, the method comprising:
a) selecting at least five (5) amino acid sequences of protein allergens as defined herein,
b) performing an alignment of the amino acid sequences of said protein allergens, and
c) determining the consensus sequence of said allergens from said alignment, wherein the selection of amino acids in the consensus sequence is based on the incidence number of the specific amino acid in each specific position in the sequence of said alignment.

In a method of the invention, when two amino acids are equally represented in a position (n) of the aligned sequence, the amino acid with the highest molecular volume according to table 3, is selected as the conserved amino acid, and furthermore when at least two amino acids, such as two, three, four, five, six, seven, eight, nine or ten amino acids, remain equally represented in a position (n) following selection of the amino acid with the highest molecular volume, according to table 3, the conserved amino acid is selected based on the physicochemical properties of amino acid according to the following groups:
group 1 [polar] comprising Asn, Gln, Ser, and Thr,
group 2 [aliphatic] comprising Val, Ala, Leu, Ile, and Met,
group 3 [basic] comprising Lys, Arg, and His,
group 4 [acidic] comprising Asp, and Glu,
group 5 [aromatic] comprising Phe, Trp, and Tyr,
group 6 comprising Pro,
group 7 comprising Gly, and
group 8 comprising Cys,
   wherein the conserved amino acid in the specific position is selected based on the selection criteria defined in Table 2.

### BRIEF DESCRIPTION OF THE FIGURES

FIG. 1 shows an IgE Immunoblot with three control nsLTPs (Sola l 7 and Sola l 3 from tomato and Ole e 7 from olive tree) and the consensus nsLTP 1 (csLTP1). Control nsLTPs and csLTP1 were tested with serum samples from 10 different patients.

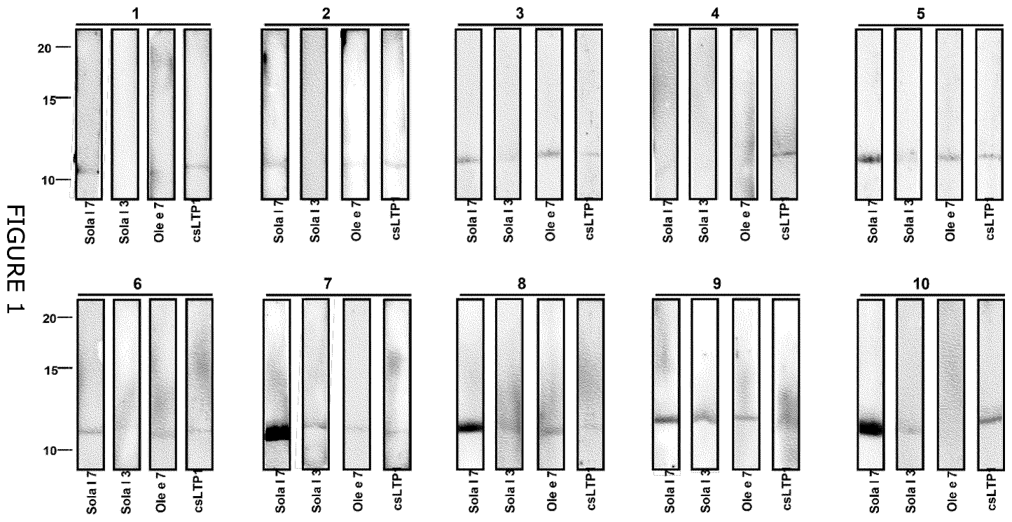

FIG. 2 shows and commercial polyclonal IgG immunoblot in which three consensus PR10 (cPR10-1, 2, 3, and 4) and a control (Bet v 1—SUMO) are recognised by a polyclonal antibody mixture raised in a rabbit against the natural PR10 Bel v 1 from Betula pendula.

FIG. 3 shows the evaluation of the CA1-specific IgG1 and IgG2a titters by ELISA in mice treated with CA1 (csLTP-1) in protein (A) and mRNA-LNP (B) format. At day 21, 42, and 63 mice serum was diluted 1:500 with PBS and antibody isotype-abundance was determined for each mouse in the group. The bars represent the average value and the bar the standard deviation of the measurements.

FIG. 4 shows the IgG1/IgG2a ratio of the mice treated with CA1 in mRNA-LNP or protein format in comparison with control mice (PBS). The bar length represents the standard deviation, and the centre of each bar is on the average value for the group.

FIG. 5 shows the evaluation of the CA1 and Pru p 3-specific IgE titters by ELISA in mice treated with CA1 in protein (A) and mRNA-LNP (B) format. Mice serum from days was diluted 1:10 with PBS and IgE abundance was determined for each mouse in the group. The bars represent the average value and the bar the standard deviation of the measurements.

FIG. 6 shows the ELISA IgE biding inhibition assay with mice serum and anaphylactic patient human serum. Inhibition was tested for both cnsLTP-1 or Pru p 3 (major peach allergen purified from the natural source). Mice serum from the 63 days from the protein and the mRNA-LNP immunised groups were diluted dilutions 1:10-1:10.000. After incubation with the mice serum, 1:10 dilution of serum from patients severely allergic to peaches was added. Points, squares, triangles pointing up and down represent the average of triplicates experimental triplicates and bars represent the standard deviation.

### DETAILED DESCRIPTION

The present invention relates to new synthetic and/or recombinant allergens, so called consensus allergens which are de novo designed based on a selection and alignment of at least 5 protein allergens, preferably at least 5 wild-type allergens. These consensus allergens can be used in allergy vaccines that has surprisingly been found to be able to alleviate a number of single or multiple allergies by exposure to a single consensus allergen. The inventors in the experimental section show that a consensus allergen is recognised by several different antibody repertoires from patients known to suffer from multiple allergies. Thus, a consensus allergen of the present invention can be used for the treatment of multiple allergies, which would under normal circumstances only be obtained from exposure to several individual allergens, such as but not limited to naturally occurring wild-type allergens.

A consensus allergen of the present invention is designed by comparing/aligning the amino acid sequences of several similar protein allergen sequences, such as wild-type protein allergens, and the consensus allergen is then constructed artificially, as a de novo amino acid sequence, based upon the conservation of residues in the compared/aligned amino acid sequences. Where the incidence number of different residues are equal, i.e., they are equally conserved, additional qualifiers are used to generate the de novo consensus sequence, such as biophysical properties of individual amino acids, sequence motifs, structural determinants etc.

A consensus allergen as described herein comprises at least 60 amino acids. Furthermore, the consensus allergen is derived from comparing the amino acid sequences of at least five (5) protein allergens, wherein said protein allergens share at least 20% amino acid sequence identity. In particular, the herein for the first time described consensus allergens are not wild-type protein allergens, they are synthetic and/or recombinant allergens.

Accordingly, the present invention relates to de novo synthetic/recombinant consensus allergens, their design, construction, production and use in medicine, such as, but not limited to, their use as allergy vaccines. In that regard, a consensus allergen of the present invention may serve several therapeutic purposes.

In consequence, the present invention in one aspect relates to allergy vaccines comprising a consensus allergen according to the present invention and/or a nucleic acid sequence encoding said consensus allergen.

Consequently, a herein disclosed allergy vaccine may be either polypeptide-based or nucleic acid-based.

### Consensus Allergen

Consensus allergens of the present invention are artificially generated allergens i.e, they are not wild-type protein allergens. A consensus allergen of the present invention is generated by sequence alignment and consensus sequence generation of a number of protein allergens, such as wild-type protein allergens. In example 1, the generation of consensus allergens is based upon allergens that are either known to be cross-recognized or that are species of the non-specific lipid transfer proteins (ns-LTP) of different origins, with a sequence identity of at least 20% over at least 90 consecutive amino acid residues. In relation to the allergen, any type of protein allergens may be used for the generation of a consensus allergen. The functionality of the consensus allergen is to elicit a broad immunological response as shown in example 2, which proves that the consensus allergen of example 1 is recognized by several individual antibody repertoires isolated from human subjects.

In the present context, an allergen is an allergenic protein that elicits an allergenic response. The term “allergen” refers to an antigen which elicits, induces, stimulates, or enhances an immune response from a cell of the immune system of an exposed animal (e.g., a human). An antigen is an allergen when the specific immune response is the development of enhanced sensitivity or a hypersensitivity to the antigen, but the antigen itself is not typically innately harmful. An allergen is therefore a particular type of antigen that can cause development of enhanced or increased sensitivity or hypersensitivity in a subject. For example, an allergen can elicit the generation of IgE antibodies in predisposed subjects.

The term “allergic response” is intended to refer to the hypersensitive immune reaction to a normally innocuous environmental substance known as an allergen. The most common mechanism of allergic reactions is the binding of IgE to the surface of mast cells, which causes asthma and other common allergic reactions.

Consensus allergens of the present invention typically comprise at least 50 amino acids and may comprise more than 1000 amino acids, such as, but not limited to, at least 60, 70, 80, 90, 100, 110, 120, 130, 150, 300, 500, 1000 1500, 2000, or 3000 amino acids, or such as between 50 and 3000 amino acids, such as between 50 and 500 amino acids, such as between 50 and 150 amino acids.

Accordingly, in embodiments, the consensus allergen comprises at least 60 amino acids and is derived from a consensus sequence of the amino acid sequences of at least five (5) protein allergens, and wherein said protein allergens share at least 20% amino acid sequence identity.

In further embodiments, the consensus allergen is derived from a consensus sequence of the amino acid sequences of at least 5, such as at least 6, 8, 10, 15, 20, 25, 30, 50, 75, 100, or 200 protein allergens.

Cross-recognized allergens are often highly similar with regard to their 3D structure, while the over-all sequence identity between the allergens is often lower. Thus, identification of allergens suitable for constructing the consensus allergen can be done using a low sequence homology as a starting point for identification of the protein allergens, i.e., a low sequence identity, such as at least 20% amino acid sequence identity.

In that regard, the consensus allergen of the present invention may be constructed from a number of wild-type protein allergens and/or from recombinant, artificial and/or synthetic protein allergens. Accordingly, a “protein allergen(s)” as defined in the present invention relates to wild-type, recombinant, artificial and/or synthetic protein allergens. The consensus allergen of the present invention may be constructed from a consensus sequence of the amino acid sequences of at least five (5) cross-recognized allergens, wherein said cross-recognized allergens share at least 20% amino acid sequence identity. The consensus allergen of the present invention may be constructed from a consensus sequence of the amino acid sequences of at least five (5) recombinant and/or synthetic allergens, wherein said recombinant and/or synthetic allergens share at least 20% amino acid sequence identity. The consensus allergen of the present invention may be constructed from a consensus sequence of the amino acid sequences of at least five (5) wild-type allergens, wherein said wild-type allergens share at least 20% amino acid sequence identity.

In embodiments, the consensus allergen of the present invention is constructed from a number of wild-type protein allergens. In another embodiment the consensus allergen of the present invention is constructed from recombinant, artificial and/or synthetic protein allergens. In additional embodiments, the consensus allergen of the present invention is constructed from a combination of wild-type protein allergens and recombinant, artificial and/or synthetic protein allergens. In additional embodiments, the consensus allergen of the present invention is constructed from a combination of cross-recognized wild-type protein allergens and recombinant, artificial and/or synthetic protein allergens.

There is, in theory, no upper limit to the number of similar sequences that may be used in the generation of the consensus allergens, as long as they share at least 20% amino acid sequence identity, over a region of at least 50 amino acids, such as at least 60, 70, 80, 90, 100, 110, 120, 130, 150, 300, or 500 amino acids.

As such, there is no upper limit to the number of similar sequences that may be used in the generation of the consensus allergens, as long as they share at least 20%, such as at least 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity, over a region of at least 50 amino acids, such as at least 60, 70, 80, 90, 100, 110, 120, 130, 150, 300, or 500 amino acids.

In that regard, a consensus allergen of the present invention may be regarded as a “synthetic and/or recombinant consensus allergen” which relates to a polypeptide-based allergen where the amino acid sequence of the synthetic and/or recombinant consensus allergen is a consensus sequence between, in theory, any number of amino acid sequences from naturally occurring wild-type protein allergens and/or from non-natural protein allergens not found in nature.

Furthermore, in embodiments, said synthetic and/or recombinant consensus allergen is encoded by a recombinant nucleic acid sequence, which is expressed by a cell using recombinant DNA/mRNA technology.

### Wild-Type Protein Allergen

The term “wild-type protein allergen” or “wild-type protein allergens” is in the current context used to describe one or more naturally occurring allergen(s) and are used interchangeably. For example, wild-type protein allergens are naturally occurring proteins, such as, but not limited to, proteins of the family of Prolamins, 2s albumins, Non-specific Lipid transfer proteins, Bifunctional α-amylase/protease inhibitors, 7/8S Albumin, 11S Albumin, Profilin, Pathogenesis-related proteins family 10, Oleosins, Endochitinases, β-1,3-Glucanases, Thaumatin-like proteins, Tropomyosins, Parvalbumins, Casein, Lipocalins, Glycosil hydrolases, and Transferrins. Non-limiting examples of protein families with known wild-type protein allergens and their origin are identified in table 1. In preferred embodiments, the consensus allergen of the invention is designed based on wild-type protein allergens belonging to one of the known protein families listed in table 1.

TABLE 1

| | Approximate | Allergens identified | Non-limiting |
|---|---|---|---|
| Protein Family | molecular mass (kDa) | from | example |
| Prolamins | 65 | Cereals | Tri a 21 |
| 2s albumins | 15-17 | Peunat, tree nuts, | Sin a 1 |
| | | mustard | |
| Non-specific Lipid | 7-9 | All type of plant food, | Pru p 3 |
| transfer proteins | | latex | |
| Bifunctional α- | 15-16 | Cereals | Sec c 28 |
| amylase/protease | | | |
| inhibitors | | | |
| 7/8S Albumin | 150-190 | Legumes, nuts, seeds | Ara h 1 |
| 11S Albumin | 60 | Peunat, tree nuts | Gly m 6 |
| Profilin | 14 | All type of plant food, | Ara h 5 |
| | | latex | |
| Pathogenesis-related | 17 | All type of plant food | Bet v 1 |
| proteins family 10 | | | |
| Oleosins | 14-17 | Legumes nuts seeds | Ara h 10 |
| Endochitinases | 25-35 | Banana, avocado, | Der f 18 |
| | | chestnut, latex, mites | |
| β-1,3-Glucanases | 25-35 | Banana, avocado, | Ole e 9 |
| | | chestnut, latex, olive | |
| | | tree | |
| Thaumatin-like | 20-25 | Kiwi, citrus and | Pru av 2 |
| proteins | | Rosaceae fruits, grape | |
| Tropomyosins | 36-38 | Crustaceans, molluscs | Pen a 1 |
| Parvalbumins | 12 | Fish and amphibians | Clu h 1 |
| Casein | 20-30 | Cow's, goat's, and | Bos d 9 |
| | | sheep's milk | |
| Lipocalins | 18 | Milk | Bos d 5 |
| Glycosil hydrolases | 14 | Cow's milk, hen's egg | Gal d 4 |
| Transferrins | 67-69 | Hen's egg, cow's milk | Gal d 3 |

In embodiments of the invention, the allergens are selected from one or more protein families. In further embodiments, the one or more protein families are selected from the group consisting of protein families identified in Table 1. In that regard, wild-type protein allergens may be selected from the group of allergens consisting of Tri a 21, Sin a 1, Pru p 3, Sec c 28, Ara h 1, Gly m 6, Ara h 5, Bet v 1, Ara h 10, Der f 18, Ole e 9, Pru av 2, Pen a 1, Clu h 1, Bos d 9, Bos d 5, Gal d 4, and Gal d 3, or homologues thereof.

In non-limiting examples, cross reactive allergens may be identified by the use of a starter sequence. A starter sequence according to the current invention is the amino acid sequence of a naturally occurring wild-type protein allergen. Non-limiting examples of naturally occurring wild-type protein allergens are allergens, such as, but not limited to, Tri a 21, Sin a 1, Pru p 3, Sec c 28, Ara h 1, Gly m 6, Ara h 5, Bet v 1, Ara h 10, Der f 18, Ole e 9, Pru av 2, Pen a 1, Clu h 1, Bos d 9, Bos d 5, Gal d 4 or Gal d 3. Accordingly, in embodiments, the amino acid sequence of a consensus allergen is obtained from the sequence comparison between the amino acid sequence of an allergen selected from the group consisting of Tri a 21, Sin a 1, Pru p 3, Sec c 28, Ara h 1, Gly m 6, Ara h 5, Bet v 1, Ara h 10, Der f 18, Ole e 9, Pru av 2, Pen a 1, Clu h 1, Bos d 9, Bos d 5, Gal d 4 and Gal d 3, and allergens with a sequence that share least 20% amino acid sequence identity thereto over at least 50 amino acids.

In further embodiments, the amino acid sequence of a consensus allergen is obtained from sequence comparison between the amino acid sequence of the allergen Tri a 21 and/or a homologue thereof, and allergens with a sequence which share least 20% amino acid sequence identity to Tri a 21 and/or a homologue thereof over a region of at least 50 amino acids. In further embodiments the amino acid sequence of a consensus allergen is obtained from sequence comparison between the amino acid sequence of the allergen Sin a 1 and/or a homologue thereof, and allergens with a sequence which share least 20% amino acid sequence identity with Sin a 1 and/or a homologue thereof over a region of at least 50 amino acids. In further embodiments the amino acid sequence of a consensus allergen is obtained from sequence comparison between the amino acid sequence of the allergen Pru p 3 and/or a homologue thereof, and allergens with a sequence which share least 20% amino acid sequence identity with Pru p 3 and/or a homologue thereof over a region of at least 50 amino acids. In further embodiments the amino acid sequence of a consensus allergen is obtained from sequence comparison between the amino acid sequence of the allergen Sec c 28 and/or a homologue thereof, and allergens with a sequence which share least 20% amino acid sequence identity with Sec c 28 and/or a homologue thereof over a region of at least 50 amino acids. In further embodiments the amino acid sequence of a consensus allergen is obtained from sequence comparison between the amino acid sequence of the allergen Ara h 1 and/or a homologue thereof, and allergens with a sequence which share least 20% amino acid sequence identity with Ara h 1 and/or a homologue thereof over a region of at least 50 amino acids. In further embodiments the amino acid sequence of a consensus allergen is obtained from sequence comparison between the amino acid sequence of the allergen Gly m 6 and/or a homologue thereof, and allergens with a sequence which share least 20% amino acid sequence identity with Gly m 6 and/or a homologue thereof over a region of at least 50 amino acids. In further embodiments the amino acid sequence of a consensus allergen is obtained from sequence comparison between the amino acid sequence of the allergen Ara h 5 and/or a homologue thereof, and allergens with a sequence which share least 20% amino acid sequence identity with Ara h 5 and/or a homologue thereof over a region of at least 50 amino acids. In further embodiments the amino acid sequence of a consensus allergen is obtained from sequence comparison between the amino acid sequence of the allergen Bet v 1 and/or a homologue thereof, and allergens with a sequence which share least 20% amino acid sequence identity with Bet v 1 and/or a homologue thereof over a region of at least 50 amino acids. In further embodiments the amino acid sequence of a consensus allergen is obtained from sequence comparison between the amino acid sequence of the allergen Ara h 10 and/or a homologue thereof, and allergens with a sequence which share least 20% amino acid sequence identity with Ara h 10 and/or a homologue thereof over a region of at least 50 amino acids. In further embodiments the amino acid sequence of a consensus allergen is obtained from sequence comparison between the amino acid sequence of the allergen Der f 18 and/or a homologue thereof, and allergens with a sequence which share least 20% amino acid sequence identity with Der f 18 and/or a homologue thereof over a region of at least 50 amino acids. In further embodiments the amino acid sequence of a consensus allergen is obtained from sequence comparison between the amino acid sequence of the allergen Ole e 9 and/or a homologue thereof, and allergens with a sequence which share least 20% amino acid sequence identity with Ole e 9 and/or a homologue thereof over a region of at least 50 amino acids. In further embodiments the amino acid sequence of a consensus allergen is obtained from sequence comparison between the amino acid sequence of the allergen Pru av 2 and/or a homologue thereof, and allergens with a sequence which share least 20% amino acid sequence identity with Pru av 2 and/or a homologue thereof over a region of at least 50 amino acids. In further embodiments the amino acid sequence of a consensus allergen is obtained from sequence comparison between the amino acid sequence of the allergen Pen a 1 and/or a homologue thereof, and allergens with a sequence which share least 20% amino acid sequence identity with Pen a 1 and/or a homologue thereof over a region of at least 50 amino acids. In further embodiments the amino acid sequence of a consensus allergen is obtained from sequence comparison between the amino acid sequence of the allergen Clu h 1 and/or a homologue thereof, and allergens with a sequence which share least 20% amino acid sequence identity with Clu h 1 and/or a homologue thereof over a region of at least 50 amino acids. In further embodiments the amino acid sequence of a consensus allergen is obtained from sequence comparison between the amino acid sequence of the allergen Bos d 9 and/or a homologue thereof, and allergens with a sequence which share least 20% amino acid sequence identity with Bos d 9 and/or a homologue thereof over a region of at least 50 amino acids. In further embodiments the amino acid sequence of a consensus allergen is obtained from sequence comparison between the amino acid sequence of the allergen Bos d 5 a and/or a homologue thereof, and allergens with a sequence which share least 20% amino acid sequence identity with Bos d 5 a and/or a homologue thereof over a region of at least 50 amino acids. In further embodiments the amino acid sequence of a consensus allergen is obtained from sequence comparison between the amino acid sequence of the allergen Gal d 4 and/or a homologue thereof, and allergens with a sequence which share least 20% amino acid sequence identity with Gal d 4 and/or a homologue thereof over a region of at least 50 amino acids. In further embodiments the amino acid sequence of a consensus allergen is obtained from sequence comparison between the amino acid sequence of the allergen Gal d 3 and/or a homologue thereof, and allergens with a sequence which share least 20% amino acid sequence identity with Gal d 3 and/or a homologue thereof over a region of at least 50 amino acids.

In that regard, a protein allergen, such as a wild-type protein allergen may be used to search for similar sequences using a BLAST alignment tool. Examples of suitable blast tools are in non-limiting examples the BLAST search for similar allergens in the ALLERGOME database (https://www.allergome.org/index.php) using the ALLERGOME database BLAST tool (https://www.allergome.org/script/tools.php?tool=blaster). Alternative and further databases for searching allergen sequences and/or performing alignment between sequences are known to the person skilled in the art.

As described in Example 1, allergens of the present invention can be identified using the naturally occurring allergen Pru p 3 as a starting point for the generation of a consensus allergen, wherein allergens are identified from a database of known allergens, based on sequence alignment as described herein. This is done by comparing a selected wild-type protein allergen and other known naturally occurring wild-type protein allergens, when identified as having a sequence which share least 20% amino acid sequence identity with Pru p 3 and/or a homologue thereof over a region of at least 50 amino acids. Accordingly, protein allergens used for the generation of the consensus allergens of the present invention may be allergens from different protein families, given their sequences are 20% identical over a region of at least 50 amino acids.

The term “cross reactivity” in general describes the ability of an antibody (such as IgE, IgG, IgA, IgM etc) present in the serum of a sensitized individual to recognize different polypeptides. Herein the cross reactivity is the ability of an antibody to recognise a peptide of an allergen which is considered to be a homologue to an allergen.

In the present invention, the term “cross-recognized”, refer to the recognition of an allergen by one or more IgEs that are otherwise specific towards other allergens. For example, a consensus allergen of the present invention may be recognised by IgEs that are otherwise specific towards one or more non-specific lipid transporter proteins, such as IgEs that recognizes the allergens Pru p 3 and Jur r 3. Cross-recognized allergens in that regard relates to the recognition of allergens by IgE due their sequence similarity and/or identity with one or more of the protein allergens, preferably sequence similarity between the consensus allergen and one or more naturally occurring wild-type protein allergens. In the current context, protein allergens, used to define the consensus allergen of the present invention may be protein allergens which share at least 20% amino acid sequence identity and potentially 80% sequence similarity, over at least 50 amino acids of the protein allergens.

In embodiments, the group of protein allergens from which the synthetic/recombinant consensus allergen is generated/designed comprises at least 5, such as at least 7, such as at least 10, such as at least 20, such as at least 50, such as at least 75, or such as at least 100 protein allergen sequences, or between 5 and 200 polypeptide sequences, such as between 10 and 200 polypeptide sequences, such as between 5 and 10 polypeptide sequences, such as between 10 and 20 polypeptide sequences, such as between 10 and 100 polypeptide sequences, such as between 10 and 200 polypeptide sequences, such as between 25 and 75 polypeptide sequences. There is theoretically no upper limit to the number of protein allergens used to generate the consensus allergen, as long as the consensus allergen retains is function, i.e., being able to be recognized by the IgEs present in the serum of allergic patients.

The person skilled in the art knows how many protein allergens are sufficient to be compared for each allergy.

In embodiments, the consensus allergen comprises at least 50 amino acids, such as at least 60, 70, 80, 90, 100, 110, 120, 150, 200, 500, 750, 1000, 1250, 1500, or 2000 amino acids, or such as between 50 and 2000, such as between 60 and 1000 or between 60 and 200 amino acids, and is derived from a consensus sequence of the amino acid sequences of at least five (5), such as at least 6, 10, 15, 20, 25, 30, 40, 50, 60, 70, 80, 90, or 100 protein allergens, or such as between 5 and 100 sequences.

The consensus allergen is not a wild-type protein allergen. Thus, although the consensus allergen is derived from a group of protein allergens, it is not 100% identical to any of the polypeptide and/or wild-type protein allergens in said group. The consensus allergen of the current invention in one embodiment differs at least 5%, 10%, 15%, 20%, 25%, 30%, 35%, 40%, 45%, 50%, 55%, 60%, 65%, 70%, 75% or 80% from any wild-type protein allergen.

In embodiments, the consensus allergen comprises at least 4 cysteine residues, such as at least 5, 6, 7, 8, 9, or 10 cysteine residues. In further embodiments, one or more of said cysteines are oxidated. In further embodiments, at least one cysteine residue, such as two, three, four or five, are substituted with non-natural amino acids. In additional embodiments, said at least one non-natural amino acid can form non-reductive intramolecular chemical bonds within the consensus allergen. In further embodiments, the 3D structure of the consensus allergen is improved or reduced by intramolecular linkages.

Non-Specific Lipid Transfer Proteins (nsLTP)

Non-specific lipid transfer proteins (nsLTPs), such as, but not limited to, Non-specific Lipid transfer protein 3 of Prunus persica (Peach), are in general small, basic proteins with a wide distribution in all orders of higher plants. Structurally, nsLTPs in general contain a conserved motif of eight (8) cysteines, linked by four disulphide bonds, and a hydrophobic cavity in which the ligand is housed.

The nsLTPs share a highly conserved structural resemblance but often share a low sequence identity, such as, as low as 20% amino acid sequence identity. The high structural similarity between the nsLTPs is one of the underlying reasons for their allergenic cross reactivity.

In embodiments, the consensus allergen comprises at least 50 amino acids and is derived from a consensus sequence of the amino acid sequences of at least five (5), such as at least 10, 20, 30, 50, 100 or 200 polypeptides selected from the group consisting of the UniProtKB identifiers A0A059SSZ0, A0A059ST23, A0A059STC4, A0A158V755, A0A161AT60, A0A1J7GK90, A0A1W5LDB3, A0A1W5LDC0, A0A1W5LDC1, A0A1W5LDC2, A0A1W5LE45, A0A1W5LG02, A0A445AL51, A0A4P1RWD8, A0A510A9S3, A0AT28, A0AT29, A0AT30, A0AT31, A0AT32, A0AT33, A0FLG4, A1E2H4, A1E2H5, A2ZAS9, A2ZAT1, A2ZDR8, A2ZHF1, A3C7Z3, A4GDQ9, A4GDS6, A4GDS7, A4GDS9, A4GDT1, A4GDT9, A4GE50, A4GE54, A4GE55, AMA5J7, A8YPK3, A9YUH6, B3A0N2, B3KN20, B6CEX8, B6CG41, B6CQU4, B6CQU6, B6CQU7, B6SGP7, B6SY96, B6T089, B6TTP1, B7VFP0, B7VFP1, B8QW29, B8QW30, B8QW32, B8QW33, B8QW34, B8QW37, B8QW40, B8QW53, B8QW56, B8QW58, B8QW69, B8QW75, B8QW95, B8QWA1, COL0I5, C4MGG9, C4MGH0, C4MGH1, C4MGH2, C5H617, D2T0A5, D2T0A6, D2T2K0, D2T2K1, D2T2K2, D3W146, D3W147, D4QD83, E6Y2L9, E6Y8S8, E7CLQ2, E7CLQ4, E7CLQ5, E7CLQ6, E7CLQ7, E7CLQ8, E7CLR2, F1AHA2, F2CY84, F2ED95, F6GXX3, F6MEX1, G8DM17, G8DM18, G8DM19, G8DM20, HM234040, HM234043, HM234051, I6QLE1, K4AYX7, K4BBD9, MOREF2, MOSPH7, M0V3U0, M1CHX3, M4QHL5, M4QL90, M4QUI6, M8C3B8, O04004, O04403, O04404, O22482, O22485, O23758, P06608, P0C088, P19656, P24296, P27056, P27631, P43217, P55958, P56252, P80274, P81402, P81430, P81651, P82007, P82534, P84160, P84161, P85205, P85206, P85894, P86333, P86809, P86838, P93224, Q0IQK9, Q0IQL2, Q0Z8V0, Q14K71, Q1JTN5, Q2PCB7, Q2PCB8, Q2PCD1, Q2PCD2, Q2QCI7, Q2QYL2, Q2QYL3, Q2RBD2, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q2XX37, Q2XX39, Q2XX47, Q2XX49, Q39382, Q40905, Q42589, Q43017, Q4A1N0, Q4A1N1, Q4PLT5, Q4PLT6, Q4PLT7, Q4PLT8, Q4PLT9, Q4PLU0, Q4VUZ0, Q53IP9, Q5GLH0, Q5IZZ5, Q5IZZ6, Q5J000, Q5J009, Q5J011, Q5J026, Q5NE26, Q5NE27, Q5NE31, Q6EV47, Q6TKQ7, Q7X9Q5, Q7XJ39, Q850K5, Q850K6, Q8GZB0, Q8H2B2, Q8L5S8, Q8RYA8, Q8VX12, Q9ATH2, Q9BMP6, Q9BPX6, Q9LED1, Q9M5X6, Q9M5X7, Q9MSX8, Q9S713, W0U0V5, B6SGP7, P55958, and P85204.

In a non-limiting example, a consensus allergen according to the present invention is generated from the nsLTP wild type allergen Pru p 3 (SEQ ID NO: 17), with the uniport identifier P81402, and wild-type protein allergens selected from the group of allergen polypeptides with an amino acid sequence according to the UniProtKB (UniprotKB: https://www.uniprot.org/uniprot/) identifiers A0A059SSZ0, A0A059ST23, A0A059STC4, B6CEX8, B6CG41, C4MGG9, C4MGH0, C4MGH1, C5H617, E6Y2L9, O04403, P0C088, P19656, P55958, P81402, P85204, P93224, Q4PLT6, Q4PLT9, Q4PLU0, Q5IZZ5, Q5IZZ6, Q5J009, Q5J011, Q5J026, Q8VX12, Q9ATH2, and W0U0V5.

In embodiments, a consensus allergen according to the present invention comprises at least 50 amino acids and is derived from a consensus sequence of the amino acid sequences of at least five (5), such as at least 6, 7, 8, 9, 10, 11, 12, 13, 14, 15, 16, 17, 18, 19, 20, 21, 22, 23, 24, 25, 26, 27, or 28, or such as 29 wild-type protein allergens selected from the group consisting of wild-type protein allergens with an amino acid sequence according to the UniProtKB identifiers of P81402, A0A059SSZ0, A0A059ST23, A0A059STC4, B6CEX8, B6CG41, C4MGG9, C4MGH0, C4MGH1, C5H617, E6Y2L9, O04403, P0C088, P19656, P55958, P81402, P85204, P93224, Q4PLT6, Q4PLT9, Q4PLU0, Q5IZZ5, Q5IZZ6, Q5J009, Q5J011, Q5J026, Q8VX12, Q9ATH2 and W0U0V5.

In another non-limiting example, a consensus allergen according to the present invention is generated from known allergenic polypeptides and nsLTPs of known cross reactivity, selected from the group of polypeptides with an amino acid sequence according to the UniProtKB identifiers O04004, E6Y8S8, B6CEX8, W0U0V5, Q9ATH2, Q8VX12, P82007, Q8RYA8, C5H617, A0AT29, Q5J026, P85894, D3W146, P81651, Q9M5X8, P82534, COL0I5, P81402, A0A059STC4, Q9M5X6, Q0Z8V0, E6Y2L9, P93224, D2T2K2, Q850K5, and P19656.

In embodiments, a consensus allergen according to the present invention comprises at least 50 amino acids and is derived from a consensus sequence of the amino acid sequences of at least five (5), such as at least 6, 7, 8, 9, 10, 11, 12, 13, 14, 15, 16, 17, 18, 19, 20, 21, 22, or 23, or such as 24 wild-type protein allergens selected from the group consisting of wild-type protein allergens with an amino acid sequence according to the UniProtKB identifiers O04004, E6Y8S8, B6CEX8, W0U0V5, Q9ATH2, Q8VX12, P82007, Q8RYA8, C5H617, A0AT29, Q5J026, P85894, D3W146, P81651, Q9MSX8, P82534, COL0I5, P81402, A0A059STC4, Q9M5X6, Q0Z8V0, E6Y2L9, P93224, D2T2K2, Q850K5, and P19656.

In another non-limiting example, a consensus allergen according to the present invention is generated from known allergenic nsLTPs selected from the group of polypeptides with an amino acid sequence according to the UniProtKB identifiers A0A059SSZ0, A0A059ST23, A0A059STC4, A0A1J7GK90, A0A4P1RWD8, A0AT29, A0AT32, A0AT33, A1E2H5, A9YUH6, B6CEX8, B6CG41, B6SGP7, B6TTP1, C5H617, D3W146, D3W147, E7CLQ2, E7CLQ4, E7CLQ5, E7CLQ6, E7CLQ7, E7CLQ8, E7CLR2, F1AHA2, F6GXX3, G8DM17, G8DM18, G8DM19, G8DM20, I6QLE1, M4QHL5, M4QL90, M4QUI6, O23758, P19656, P81402, P81651, P82534, P85894, Q0Z8V0, Q2QCI7, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q43017, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q6EV47, Q6TKQ7, Q850K5, Q850K6, Q8H2B2, Q8L5S8, Q8RYA8, Q8VX12, Q9LED1, Q9M5X6, Q9M5X7, Q9M5X8, and W0U0V5.

In embodiments, a consensus allergen according to the present invention comprises at least 50 amino acids and is derived from a consensus sequence of the amino acid sequences of at least five (5), such as at least 6, 10, 15, 20, 25, 30, 40, 50, 60 or 70 wild-type protein allergens selected from the group consisting of wild-type protein allergens with an amino acid sequence according to the UniProtKB identifier A0A059SSZ0, A0A059ST23, A0A059STC4, A0A1J7GK90, A0A4P1RWD8, A0AT29, A0AT32, A0AT33, A1E2H5, A9YUH6, B6CEX8, B6CG41, B6SGP7, B6TTP1, C5H617, D3W146, D3W147, E7CLQ2, E7CLQ4, E7CLQ5, E7CLQ6, E7CLQ7, E7CLQ8, E7CLR2, F1AHA2, F6GXX3, G8DM17, G8DM18, G8DM19, G8DM20, I6QLE1, M4QHL5, M4QL90, M4QUI6, O23758, P19656, P81402, P81651, P82534, P85894, Q0Z8V0, Q2QCI7, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q43017, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q6EV47, Q6TKQ7, Q850K5, Q850K6, Q8H2B2, Q8L5S8, Q8RYA8, Q8VX12, Q9LED1, Q9M5X6, Q9M5X7, Q9M5X8, and W0U0V5.

In another non-limiting example, a consensus allergen according to the present invention is generated from alignment of the wild type nsLTP allergen Jur r3 (SEQ ID NO: 18) with the uniport identifier C5H617 and wild-type protein allergens are selected from the group of allergen polypeptides with an amino acid sequence according to the UniProtKB identifiers A0A059SSZ0, A0A059ST23, A0A059STC4, A0A158V755, A0A161AT60, A0A1J7GK90, A0A1W5LDB3, A0A1W5LDC0, A0A1W5LDC1, A0A1W5LDC2, A0A1W5LE45, A0A1W5LG02, A0A445AL51, A0A4P1RWD8, A0A510A9S3, A0AT28, A0AT29, A0AT30, A0AT31, A0AT32, A0AT33, A1E2H4, A1E2H5, A2ZAS9, A2ZAT1, A2ZDR8, A2ZHF1, A3C7Z3, A8YPK3, A9YUH6, B6CEX8, B6CG41, B6CQU4, B6CQU6, B6CQU7, B6SGP7, B6SY96, B6T089, B6TTP1, B8QW29, B8QW30, B8QW32, B8QW33, B8QW34, B8QW37, B8QW40, B8QW53, B8QW56, B8QW58, B8QW69, B8QW75, B8QW95, B8QWA1, COL0I5, C4MGG9, C4MGH0, C4MGH1, C4MGH2, C5H617, D2T0A5, D2T0A6, D2T2K0, D2T2K1, D2T2K2, D3W146, D3W147, D4QD83, E6Y2L9, E6Y8S8, E7CLQ2, E7CLQ4, E7CLQ5, E7CLQ6, E7CLQ7, E7CLQ8, E7CLR2, F1AHA2, F2CY84, F2ED95, F6GXX3, F6MEX1, G8DM17, G8DM18, G8DM19, G8DM20, I6QLE1, M0V3U0, M1CHX3, M4QHL5, M4QL90, M4QUI6, O04004, O04403, O04404, O22482, O22485, O23758, O65091, P19656, P24296, P27056, P27631, P43217, P55958, P81402, P81651, P82007, P82534, P85206, P85894, P86137, P93224, Q0IQK9, Q0Z8V0, Q14K71, Q1JTN5, Q2PCB7, Q2PCB8, Q2PCD1, Q2PCD2, Q2QCI7, Q2QYL2, Q2QYL3, Q2RBD2, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q2XX37, Q2XX39, Q2XX47, Q2XX49, Q39382, Q40905, Q42589, Q43017, Q4A1N0, Q4A1N1, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q5NE26, Q5NE27, Q5NE31, Q6EV47, Q6TKQ7, Q7XJ39, Q850K5, Q850K6, Q8GZB0, Q8H2B2, Q8L5S8, Q8RYA8, Q8VX12, Q9ATH2, Q9LED1, Q9M5X6, Q9M5X7, Q9M5X8, Q9S713, and W0U0V5.

In embodiments, a consensus allergen according to the present invention comprises at least 50 amino acids and is derived from a consensus sequence of the amino acid sequences of at least five (5), such as at least 6, 10, 15, 20, 25, 30, 40, 50, 60, 70, 80, 90 or 100 wild-type protein allergens, selected from the group consisting of wild-type protein allergens with an amino acid sequence according to UniProtKB identifier of C5H617, A0A059SSZ0, A0A059ST23, A0A059STC4, A0A158V755, A0A161AT60, A0A1J7GK90, A0A1W5LDB3, A0A1W5LDC0, A0A1W5LDC1, A0A1W5LDC2, A0A1W5LE45, A0A1W5LG02, A0A445AL51, A0A4P1RWD8, A0A510A9S3, A0AT28, A0AT29, A0AT30, A0AT31, A0AT32, A0AT33, A1E2H4, A1E2H5, A2ZAS9, A2ZAT1, A2ZDR8, A2ZHF1, A3C7Z3, A8YPK3, A9YUH6, B6CEX8, B6CG41, B6CQU4, B6CQU6, B6CQU7, B6SGP7, B6SY96, B6T089, B6TTP1, B8QW29, B8QW30, B8QW32, B8QW33, B8QW34, B8QW37, B8QW40, B8QW53, B8QW56, B8QW58, B8QW69, B8QW75, B8QW95, B8QWA1, COL0I5, C4MGG9, C4MGH0, C4MGH1, C4MGH2, C5H617, D2T0A5, D2T0A6, D2T2K0, D2T2K1, D2T2K2, D3W146, D3W147, D4QD83, E6Y2L9, E6Y8S8, E7CLQ2, E7CLQ4, E7CLQ5, E7CLQ6, E7CLQ7, E7CLQ8, E7CLR2, F1AHA2, F2CY84, F2ED95, F6GXX3, F6MEX1, G8DM17, G8DM18, G8DM19, G8DM20, I6QLE1, M0V3U0, M1CHX3, M4QHL5, M4QL90, M4QUI6, O04004, O04403, O04404, O22482, O22485, O23758, O65091, P19656, P24296, P27056, P27631, P43217, P55958, P81402, P81651, P82007, P82534, P85206, P85894, P86137, P93224, Q0IQK9, Q0Z8V0, Q14K71, Q1JTN5, Q2PCB7, Q2PCB8, Q2PCD1, Q2PCD2, Q2QCI7, Q2QYL2, Q2QYL3, Q2RBD2, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q2XX37, Q2XX39, Q2XX47, Q2XX49, Q39382, Q40905, Q42589, Q43017, Q4A1N0, Q4A1N1, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q5NE26, Q5NE27, Q5NE31, Q6EV47, Q6TKQ7, Q7XJ39, Q850K5, Q850K6, Q8GZB0, Q8H2B2, Q8L5S8, Q8RYA8, Q8VX12, Q9ATH2, Q9LED1, Q9M5X6, Q9M5X7, Q9M5X8, Q9S713, and W0U0V5.

### Peach-Cypress Consensus Allergens

The present invention also relates to vaccines comprising or encoding specific consensus allergens. In Example 3 and 4, it is disclosed that synthetic/recombinant consensus allergens can be designed and produced so that they are recognised by IgEs present in the samples of 10 different allergic patients, demonstrating that the consensus design conserves the IgE reactive epitopes present in the natural wild-type protein allergens, such as in nsLTPs.

Thus, the invention also relates to isolated consensus allergens generated according to the method of the present invention.

In embodiments, the invention relates to a consensus allergen with an amino acid sequence selected from the group consisting of SEQ ID NOs 1-4 and functional homologues thereof with an amino acid sequence which is at least 80%, 85%, 90%, 95%, 97%, 98%, or 99% identical to any one of SEQ ID NOs 1-4.

In the present context, functional homologues of consensus allergens share a conserved IgE reactive epitope and/or elicit a similar immunogenic function, such as providing an IgG response which is at least 50%, such as 75%, 85%, 95%, 100%, 110%, or 150% of the IgG response obtained by the consensus allergen in a host, or such as providing an IgG response which is at least 50%, such as 75%, 85%, 95%, 100%, 110%, or 150% of the IgE response obtained by the consensus allergen in a host.

In some embodiments, the allergy vaccine comprises a nucleic acid sequence encoding a consensus allergen according to SEQ ID NO: 1, or a functional homologue thereof with an amino acid sequence which is at least 80%, 85%, 90%, 95%, 97%, 98%, or 99% identical to SEQ ID NOs 1.

In embodiments, the allergy vaccine comprises a nucleic acid sequence encoding a consensus allergen according to SEQ ID NO: 2, or a functional homologue thereof with an amino acid sequence which is at least 80%, 85%, 90%, 95%, 97%, 98%, or 99% identical to SEQ ID Nos 2.

In some embodiments, the allergy vaccine comprises a nucleic acid sequence encoding a consensus allergen according to SEQ ID NO: 3, or a functional homologue thereof with an amino acid sequence which is at least 80%, 85%, 90%, 95%, 97%, 98%, or 99% identical to SEQ ID NOs 3.

In embodiments, the allergy vaccine comprises a nucleic acid sequence encoding a consensus allergen according to SEQ ID NO: 4, or a functional homologue thereof with an amino acid sequence which is at least 80%, 85%, 90%, 95%, 97%, 98%, or 99% identical to SEQ ID NOs 4.

In embodiments, the allergy vaccine comprises a nucleic acid sequence encoding a consensus allergen according to SEQ ID NO: 1, or a functional homologue thereof with an amino acid sequence which is at least 80%, 85%, 90%, 95%, 97%, 98%, or 99% identical to SEQ ID NOs 1.

In embodiments, the allergy vaccine comprises a nucleic acid sequence encoding a consensus allergen according to SEQ ID NO: 2, or a functional homologue thereof with an amino acid sequence which is at least 80%, 85%, 90%, 95%, 97%, 98%, or 99% identical to SEQ ID NOs 2.

In embodiments, the allergy vaccine comprises a nucleic acid sequence encoding a consensus allergen according to SEQ ID NO: 3, or a functional homologue thereof with an amino acid sequence which is at least 80%, 85%, 90%, 95%, 97%, 98%, or 99% identical to SEQ ID NOs 3.

In embodiments, the allergy vaccine comprises a nucleic acid sequence encoding a consensus allergen according to SEQ ID NO: 4, or a functional homologue thereof with an amino acid sequence which is at least 80%, 85%, 90%, 95%, 97%, 98%, or 99% identical to SEQ ID NOs 4.

Accordingly in embodiments, the invention relates to an isolated nucleic acid molecule comprising a nucleic acid sequence encoding a consensus allergen with an amino acid sequence selected from the group consisting of SEQ ID NOs 1-4 and functional homologues thereof with an amino acid sequence which is at least 80%, 85%, 90%, 95%, 97%, 98%, or 99% identical to any one of SEQ ID NOs 1-4.

In further embodiments, the invention relates to an isolated consensus allergen which comprises an amino acid sequence selected from the group consisting of SEQ ID NOs 1-4 and functional homologues thereof with an amino acid sequence which is at least 80%, 85%, 90%, 95%, 97%, 98%, or 99% identical to any one of SEQ ID NOs 1-4.

In embodiments, the consensus allergen of SEQ ID NO: 1 is generated from the wild-type protein allergens selected from allergen polypeptides with an amino acid sequence according to the UniProtKB (UniprotKB: https://www.uniprot.org/uniprot/) identifiers A0A059SSZ0, A0A059ST23, A0A059STC4, B6CEX8, B6CG41, C4MGG9, C4MGH0, C4MGH1, C5H617, E6Y2L9, O04403, P0C088, P19656, P55958, P81402, P85204, P93224, Q4PLT6, Q4PLT9, Q4PLU0, Q5IZZ5, Q5IZZ6, Q5J009, Q5J011, Q5J026, Q8VX12, Q9ATH2, and W0U0V5.

In embodiments, the consensus allergen of SEQ ID NO: 2 is generated from known allergenic polypeptides and nsLTPs of known cross-reactivity with an amino acid sequence according to the UniProtKB identifiers O04004, E6Y8S8, B6CEX8, W0U0V5, Q9ATH2, Q8VX12, P82007, Q8RYA8, C5H617, A0AT29, Q5J026, P85894, D3W146, P81651, Q9M5X8, P82534, COL0I5, P81402, A0A059STC4, Q9M5X6, Q0Z8V0, E6Y2L9, P93224, D2T2K2, Q850K5, and P19656.

In embodiments, the consensus allergen of SEQ ID NO: 3 is generated from known allergenic nsLTPs with an amino acid sequence according to the UniProtKB identifiers A0A059SSZ0, A0A059ST23, A0A059STC4, A0A1J7GK90, A0A4P1RWD8, A0AT29, A0AT32, A0AT33, A1E2H5, A9YUH6, B6CEX8, B6CG41, B6SGP7, B6TTP1, C5H617, D3W146, D3W147, E7CLQ2, E7CLQ4, E7CLQ5, E7CLQ6, E7CLQ7, E7CLQ8, E7CLR2, F1AHA2, F6GXX3, G8DM17, G8DM18, G8DM19, G8DM20, I6QLE1, M4QHL5, M4QL90, M4QUI6, O23758, P19656, P81402, P81651, P82534, P85894, Q0Z8V0, Q2QCI7, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q43017, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q6EV47, Q6TKQ7, Q850K5, Q850K6, Q8H2B2, Q8L5S8, Q8RYA8, Q8VX12, Q9LED1, Q9M5X6, Q9M5X7, Q9MSX8, and W0U0V5.

In embodiments, the consensus allergen of SEQ ID NO: 4 is generated based on wild-type protein allergens selected from the group consisting of allergens with an amino acid sequence according to the UniProtKB identifiers A0A059SSZ0, A0A059ST23, A0A059STC4, A0A158V755, A0A161AT60, A0A1J7GK90, A0A1W5LDB3, A0A1W5LDC0, A0A1W5LDC1, A0A1W5LDC2, A0A1W5LE45, A0A1W5LG02, A0A445AL51, A0A4P1RWD8, A0A510A9S3, A0AT28, A0AT29, A0AT30, A0AT31, A0AT32, A0AT33, A1E2H4, A1E2H5, A2ZAS9, A2ZAT1, A2ZDR8, A2ZHF1, A3C7Z3, A8YPK3, A9YUH6, B6CEX8, B6CG41, B6CQU4, B6CQU6, B6CQU7, B6SGP7, B6SY96, B6T089, B6TTP1, B8QW29, B8QW30, B8QW32, B8QW33, B8QW34, B8QW37, B8QW40, B8QW53, B8QW56, B8QW58, B8QW69, B8QW75, B8QW95, B8QWA1, COL0I5, C4MGG9, C4MGH0, C4MGH1, C4MGH2, C5H617, D2T0A5, D2T0A6, D2T2K0, D2T2K1, D2T2K2, D3W146, D3W147, D4QD83, E6Y2L9, E6Y8S8, E7CLQ2, E7CLQ4, E7CLQ5, E7CLQ6, E7CLQ7, E7CLQ8, E7CLR2, F1AHA2, F2CY84, F2ED95, F6GXX3, F6MEX1, G8DM17, G8DM18, G8DM19, G8DM20, I6QLE1, M0V3U0, M1CHX3, M4QHL5, M4QL90, M4QUI6, O04004, O04403, O04404, O22482, O22485, O23758, O65091, P19656, P24296, P27056, P27631, P43217, P55958, P81402, P81651, P82007, P82534, P85206, P85894, P86137, P93224, Q0IQK9, Q0Z8V0, Q14K71, Q1JTN5, Q2PCB7, Q2PCB8, Q2PCD1, Q2PCD2, Q2QCI7, Q2QYL2, Q2QYL3, Q2RBD2, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q2XX37, Q2XX39, Q2XX47, Q2XX49, Q39382, Q40905, Q42589, Q43017, Q4A1N0, Q4A1N1, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q5NE26, Q5NE27, Q5NE31, Q6EV47, Q6TKQ7, Q7XJ39, Q850K5, Q850K6, Q8GZB0, Q8H2B2, Q8L5S8, Q8RYA8, Q8VX12, Q9ATH2, Q9LED1, Q9M5X6, Q9M5X7, Q9M5X8, Q9S713, and W0U0V5.

### Birch-Apple Syndrome Consensus Allergens

The present invention also relates to vaccines comprising or encoding specific consensus allergens. In Example 1 and 2, it is disclosed that synthetic/recombinant consensus allergens can be designed and produced so that they are recognised by polyclonal IgGs raised against the natural wild-type protein allergens, such as in Bet vi allergen from Betula pendula.

In embodiments, the invention relates to a consensus allergen with an amino acid sequence selected from the group consisting of SEQ ID NOs 20-23 and functional homologues thereof with an amino acid sequence which is at least 80%, 85%, 90%, 95%, 97%, 98%, or 99% identical to any one of SEQ ID NOs 20-23.

In some embodiments, the allergy vaccine comprises a nucleic acid sequence encoding a consensus allergen according to SEQ ID NO:20, or a functional homologue thereof with an amino acid sequence which is at least 80%, 85%, 90%, 95%, 97%, 98%, or 99% identical to SEQ ID NOs 20.

In embodiments, the allergy vaccine comprises a nucleic acid sequence encoding a consensus allergen according to SEQ ID NO: 21, or a functional homologue thereof with an amino acid sequence which is at least 80%, 85%, 90%, 95%, 97%, 98%, or 99% identical to SEQ ID NO 21.

In some embodiments, the allergy vaccine comprises a nucleic acid sequence encoding a consensus allergen according to SEQ ID NO: 22, or a functional homologue thereof with an amino acid sequence which is at least 80%, 85%, 90%, 95%, 97%, 98%, or 99% identical to SEQ ID NO 22.

In embodiments, the allergy vaccine comprises a nucleic acid sequence encoding a consensus allergen according to SEQ ID NO: 23, or a functional homologue thereof with an amino acid sequence which is at least 80%, 85%, 90%, 95%, 97%, 98%, or 99% identical to SEQ ID NO 23.

In embodiments, the allergy vaccine comprises a nucleic acid sequence encoding a consensus allergen according to SEQ ID NO: 20, or a functional homologue thereof with an amino acid sequence which is at least 80%, 85%, 90%, 95%, 97%, 98%, or 99% identical to SEQ ID NOs 20.

In embodiments, the allergy vaccine comprises a nucleic acid sequence encoding a consensus allergen according to SEQ ID NO: 21, or a functional homologue thereof with an amino acid sequence which is at least 80%, 85%, 90%, 95%, 97%, 98%, or 99% identical to SEQ ID NO 21.

In embodiments, the allergy vaccine comprises a nucleic acid sequence encoding a consensus allergen according to SEQ ID NO: 22, or a functional homologue thereof with an amino acid sequence which is at least 80%, 85%, 90%, 95%, 97%, 98%, or 99% identical to SEQ ID NO 22.

In embodiments, the allergy vaccine comprises a nucleic acid sequence encoding a consensus allergen according to SEQ ID NO: 4, or a functional homologue thereof with an amino acid sequence which is at least 80%, 85%, 90%, 95%, 97%, 98%, or 99% identical to SEQ ID NO 23.

Accordingly in embodiments, the invention relates to an isolated nucleic acid molecule comprising a nucleic acid sequence encoding a consensus allergen with an amino acid sequence selected from the group consisting of SEQ ID NOs 20-23 and functional homologues thereof with an amino acid sequence which is at least 80%, 85%, 90%, 95%, 97%, 98%, or 99% identical to any one of SEQ ID NOs 20-23.

In further embodiments, the invention relates to an isolated consensus allergen which comprises an amino acid sequence selected from the group consisting of SEQ ID NOs 20-23 and functional homologues thereof with an amino acid sequence which is at least 80%, 85%, 90%, 95%, 97%, 98%, or 99% identical to any one of SEQ ID NOs 20-23.

In embodiments, the consensus allergen of SEQ ID NO: 20 is generated based on wild-type protein allergens selected from the group consisting of allergens with an amino acid sequence according to the UniProtKB identifiers B6CQR8, Q2I6V8, B6CQR7, B5KVN9, B5KVP1, O22521, Q9S7M5, Q9SYV4, A0AUB1, F6M053, Q9SYV9, Q941P5, Q9SYV3, Q8L6K9, Q9SYV2, Q9SYV5, Q9SYW3, Q9SYV8, Q9SYV6, Q9SYV7, Q941P6, Q4VPL0, F5CEW9, F6LXS7, Q4VPK7, A0AUB7, B6CQS1, A0AUB6, A0AUB8, A0AUC8, Q43552, M5XFW3, Q4VPK6, B6CQR9, Q43550, Q43551, B6CQS2, Q84LA7, Q5VJQ7, Q4VPI9, B6CQS5, Q4VPI0, Q4VPI6, Q5VJR0, B6CQS6, Q4VPJ0, Q941P8, Q43549, Q5VJR1, Q4VPI3, Q4VPI7, G8H6R0, Q4VPH9, E4Z8P8, Q4VPJ5, H9NJ57, Q5VJQ8, Q941P7, B6CQT0, Q5VJQ9, B6CQS4, B6CQS3, M5XTC6, H9NJ55, B6CQT1, A0A1J0RET5, Q6QHU2, A0AU76, B6CQS7, B7VFN6, H9NJ56, H9NJ58, B6CQS9, Q6QHU3, Q4VPK5, Q9ZRU8, A0AUE3, A0AU75, Q6QHU1, B7TWE7, B0B0L9, Q5VJR5, B0B0M5, B7TWE6, B7TWE8, B0B0L6, A0AUG8, Q4VPJ8, B0B0L5, Q4VPJ9, Q5VJR2, A0AUF9, Q5VJR4, F6LWG3, F6LWG8, D7SY83, A5C113, F6LWG7, A0AUG9, Q93YH9, Q5VJR3, Q4VPK0, F6LWF9, A0AUF7, Q4VPJ7, ASB0T9, G8E012, D7SY82, H9NJ59, F6LWG6, F6KDF1, A0AUH0, A5C112, A0AU70, A0AU71, Q4VPJ1, F6H6U9, B6RQS2, Q39454, A5CAV3, Q4VPJ3, B7TWE4, A5AQ75, Q9FS42, B7TWE3, D7SY74, B7TWE5, A0A2H5CUG2, Q39415, F6H6U5, Q9SWR4, B6RQS3, Q9FPK3, Q39427, Q9FPK4, Q9FPK2, B9RTC1, H9NJ54, O23747, A8W7B6, O23749, Q39429, Q39428, B6RQS1, Q9LEP0, Q39453, Q39420, Q0QLT4, Q9ZS38, Q96370, C0IW09, C0IW05, C0IW03, C0IW04, C0IVZ8, C0IW07, C0IW00, Q0QLT5, Q9ZS39, O23752, Q9SCH6, O23754, Q0Z8U9, C0IW10, O24642, Q9SYW2, Q9SCI2, Q9SCH5, Q42499, Q546U3, O23753, O23748, Q96371, Q96366, Q39426, Q9SCH8, Q39430, Q96382, O23751, Q9SCH9, Q546V0, Q9SYW0, D1YSM4, Q96378, Q9SCI0, Q96365, C0IVT2, Q0QKX4, C0IVS9, C0IVS8, Q0QLV3, Q9AYS2, Q0QLS7, Q39431, Q96367, Q9AYS4, Q9SCI3, C0IVT9, C0IVT4, Q9SYW1, B9RTC5, Q96381, Q0QLV6, Q9AYS3, D1YSM5, E9M219, B6RQR9, B6RQR6, Q96377, B6RQR7, E2GL17, B6RQS0, B6RQR8, Q96503, Q96379, Q39425, K4CWC4, Q96501, E9M220, C0IVZ2, Q96380, C0IVZ5, Q0QLU7, C0IVZ0, C0IVY9, C0IVZ3, C0IVY2, C0IVY6, C0IVY7, Q0QKW8, C0IVZ1, Q0QLT9, Q96368, Q546V1, O23746, O23750, C0IVP0, Q0QKX7, Q0QLT3, C0IVP2, C0IVP5, C0IVQ6, C0IVP3, Q0QLW3, C0IVP6, C0IVQ7, C0IVR6, C0IVP1, C0IVP4, C0IVR1, C0IVR7, Q0QLS9, C0IVQ4, Q0QLT1, C0IVR2, C0IVR5, Q0QLS8, C0IVP8, Q0QLW1, C0IVS1, C0IVS3, Q0QLV9, Q0QLV8, Q0QLW0, C0IVR8, C0IVR9, C0IVR4, C0IVP9, C0IVQ1, C0IVQ2, Q0QKX5, C0IVR0, Q0QLT0, C0IVQ3, C0IVS2, C0IVS4, C0IVS0, C0IVQ8, C0IVQ9, C0IVR3, C0IVT8, Q0QLV2, C0IVU1, C0IVU0, C0IVS6, C0IVU4, C0IVS5, C0IVU2, C0IVT7, C0IVU5, Q0QLV0, C0IVT0, Q0QLV5, C0IVT6, Q0QLS6, C0IVX8, C0IVX7, C0IVY4, C0IVW8, Q0QLS2, C0IVU3, C0IVW6, C0IVW2, C0IVY0, C0IVX2, C0IVV4, C0IVV6, Q0QLS5, C0IVX9, C0IVV8, Q0QLU8, C0IVT5, C0IVW0, Q0QKX2, C0IVX4, C0IVV5, C0IVW7, C0IVW9, C0IVX3, C0IVY3, C0IVV2, C0IVX6, C0IVV1, C0IVU9, C0IVW3, C0IVV0, Q0QKW9, Q0QLU2, C0IVV3, C0IVW5, C0IVU7, C0IVU6, Q0QKX1, C0IW11, Q9SCI1, Q9SCH7 and C0IVZ4.

In embodiments, the consensus allergen of SEQ ID NO: 21 is generated based on wild-type protein allergens selected from the group consisting of allergens with an amino acid sequence according to the UniProtKB identifiers P43211, Q9SYW3, Q941P6, Q9SYV7, Q9SYV2, Q9SYV6, Q9SYV5, F5CEW9, Q9SYV8, P43211, O65200, Q9S7M5, Q40280, Q9SYV4, Q40280, A0AUB1, Q941P5, Q9SYV3, F6M053, Q9SYV9, Q8L6K9, O24248, B5KVN9, B5KVP1, B6CQR8, Q2I6V8, B6CQR7, O22521, B7VFN6, Q84LA7, Q4VPI9, Q941P8, Q5VJR0, Q4VPI6, Q4VPJ0, Q256S4, Q5VJR1, Q4VPI3, Q4VPJ5, Q4VPI0, Q5VJQ9, Q43549, Q256S7, Q3T923, Q941P7, Q5VJQ8, Q256S2, Q5VJR5, Q4VPI7, Q256S6, B6CQS3, Q5VJR2, B0B0L6, A0AUF9, Q4VPJ8, B0B0L5, A0AUG8, M5XFW3, B6CQS4, Q4VPK5, Q4VPJ9, F6LWG3, F6LWG8, A0AUE3, A0AU76, A0AU75, F6LWG7, B6CQS1, B0B0L9, A0A1J0RET5, Q5VJR3, Q4VPH9, A0AUG9, B0B0M5, Q4VPK0, A0AUF7, E4Z8P8, B6CQS2, F6LWF9, Q4VPJ7, G8E012, B6CQS5, Q5VJR4, B6CQR9, O50001, F6LWG6, ref:23394, D0E0C7, A0AUB7, B6CQS6, A0AUH0, Q4VPL0, Q5VJQ7, Q4VPK7, F6LXS7, A0AUC8, ref:23394, ref:23394, G8H6R0, A0AUB8, A0AUB6, H9NJ57, D0E0C6, ref:23394, Q43552, Q9ZRU8, Q43551, ref:23394, Q43550, B6CQT0, H9NJ59, Q4VPK6, ref:23394, F6KDF1, H9NJ55, ref:23394, H9NJ58, Q93YH9, H9NJ56, ref:23394, ref:23394, M5XTC6, B6CQT1, Q6QHU2, ref:23394, B6CQS7, ref:23394, ref:13687, ref:23394, ref:23394, B6CQS9, ref:23394, B7TWE7, Q6QHU3, B7TWE6, Q6QHU1, Q4VPJ1, A0AU70, B7TWE8, A0AU71, A5C113, A5B0T9, D7SY82, ref:23394, A5C112, D7SY83, Q4VPJ3, Q39454, Q39415, Q9SWR4, A8W7B6, Q9FPK4, Q9FPK2, F6H6U9, Q9FPK3, A5CAV3, B7TWE3, F6H6U5, B7TWE5, A5AQ75, B6RQS2, B7TWE4, Q9FS42, Q39427, D7SY74, A0A2H5CUG2, P43186, Q9LEP0, Q39429, Q39428, O23747, O23749, Q39420, Q0Z8U9, P43176, B6RQS3, P43184, H9NJ54, P45431, Q0QLT4, Q39453, Q9ZS38, C0IW05, C0IW03, C0IW04, C0IVZ8, C0IW07, C0IW00, Q0QLT5, B9RTC1, ref:19841, C0IW09, Q9SYW2, Q9SCH6, C0IW10, Q9SCH5, B6RQS1, Q9ZS39, Q39430, B9RTC5, Q9SCH9, Q96370, O23748, O23751, O23752, Q9SYW0, O23754, Q9AYS2, Q9SCI0, Q39426, Q96365, O24642, P38948, P43180, Q08407, P43177, Q42499, Q96366, O23746, Q546U3, P15494, P43183, Q96371, Q9SCH8, O23753, P43178, Q9AYS4, Q9SCI2, Q9SCW3, C0IVS9, C0IVS8, Q0QLV3, Q96367, P43179, Q0QLU7, Q39425, P43185, C0IVX8, C0IVX7, Q96368, C0IVY2, Q96382, C0IVV8, Q0QLU8, C0IVT2, Q0QKX4, Q9AYS3, C0IVW2, C0IVY0 and C0IVX2.

In embodiments, the consensus allergen of SEQ ID NO: 22 is generated based on wild-type protein allergens selected from the group consisting of allergens with an amino acid sequence according to the UniProtKB identifiers Q546U3, P15494, Q96366, O24642, Q42499, Q546V0, P15494, Q9SCH8, O23752, Q96371, Q96370, Q9SCI0, Q9SCH9, Q96365, Q9SYW1, Q96367, Q9SCI3, Q9SYW0, Q9SCI2, O23753, Q9AYS2, O23754, O23751, Q96368, P43183, Q9AYS3, Q39431, P43177, Q39426, P43179, P43180, Q9SYW2, Q9SCH5, Q9ZS39, O23748, ref:19841, P43185, Q546V1, P43178, Q9AYS4, ref:10368, O23746, Q9SCH6, O23750, C0IVP0, Q0QKX7, Q0QLT3, C0IVP2, C0IVP5, C0IVQ6, C0IVP3, Q0QLW3, C0IVP6, C0IVQ7, Q9ZS38, C0IVR6, C0IVP1, C0IVP4, C0IVR1, C0IVR7, Q0QLS9, C0IVQ4, Q0QLT1, C0IVR2, C0IVR5, ref:10368, Q0QLS8, Q39429, C0IVP8, Q0QLW1, C0IVS1, Q39428, P43186, C0IVS3, Q0QLV9, Q0QLV8, Q0QLW0, Q39427, C0IVR8, C0IVR9, C0IVR4, C0IVP9, C0IVQ1, C0IVQ2, Q0QKX5, C0IVR0, Q0QLT0, O23749, C0IVQ3, P45431, C0IVS2, C0IVS4, P43184, O23747, C0IVS0, P43176, C0IVQ8, C0IVQ9, C0IVR3, Q9LEP0, Q39430, Q39420, Q39453, C0IVT2, Q0QKX4, C0IVT9, C0IVT4, Q39425, C0IVS9, C0IVS8, Q0QLV3, Q0QLV6, C0IVT8, Q0QLV2, C0IVU1, C0IVU0, C0IVS6, P38948, C0IVU4, Q0QLS7, C0IVS5, C0IVU2, C0IVT7, C0IVU5, Q0QLV0, C0IVT0, Q0QLV5, C0IVT6, Q0QLS6, C0IVX8, C0IVX7, C0IVY4, C0IVW8, Q0QLS2, C0IVU3, C0IVW6, C0IVW2, C0IVY0, C0IVX2, C0IW4, C0IW6, Q0QLS5, C0IVX9, C0IVV8, Q0QLU8, C0IVT5, C0IVW0, Q0QKX2, C0IVX4, Q0QLU7, C0IVV5, C0IVW7, Q96382, C0IVW9, C0IVX3, C0IVY2, C0IVY3, C0IW2, C0IVX6, C0IVV1, C0IVU9, C0IVW3, C0IVV0, Q0QKW9, Q0QLU2, C0IVV3, C0IVW5, C0IVU7, C0IVU6, Q0QKX1, P38950, Q96381, C0IW11, Q96378, Q96377, B6RQR6, B6RQR9, B6RQR8, B6RQR7, E2GL17, Q08407, B6RQS0, Q96379, Q08407, Q96503, Q08407, P38949, P38949, Q96501, Q9SCI1, Q39415, Q08407, Q9SCH7, Q96380, H9NJ55, H9NJ58, Q39454, H9NJ59, H9NJ57, Q9ZRU8, H9NJ56, Q9SWR4, C0IW05, C0IW03, C0IW04, C0IVZ8, C0IW07, C0IW00, Q0QLT5, C0IVZ5, C0IW09, Q9FPK2, Q93YH9, Q0QLT4, Q9FPK4, Q9FPK3, C0IVZ1, Q0QLT9, C0IVZ3, C0IVZ0, C0IVY9, C0IW10, C0IVY6, C0IVY7, Q0QKW8, A8W7B6, B7TWE8, B7TWE7, C0IVZ4, C0IVZ2, B7TWE6, Q43550, A0A1J0RET5, Q43551, Q43552, A0AUB6, Q4VPL0, F6LXS7, A0AUB7, A0AUB8, B6CQS5, B6CQS6, O24248, Q43549, D7SY82, ASB0T9, O50001, A5C113, B6CQR8 and Q2I6V8.

In embodiments, the consensus allergen of SEQ ID NO: 23 is generated based on wild-type protein allergens selected from the group consisting of allergens with an amino acid sequence according to the UniProtKB identifiers D1YSM4, D1YSM5, ASB0T9, A5C112, D7SY82, D7SY83, F6H6U9, O50001, A5C113, B6CQR9, F6LXS7, A0AUB7, Q4VPL0, A5CAV3, M5XFW3, A0AUB8, Q43551, A0AUB6, Q43552, Q43550, F6H6U5, H9NJ59, A5AQ75, Q93YH9, B6RQS2, B6CQS5, B6CQS6, D0E0C6, Q9ZRU8, Q5VJQ7, Q9FS42, B6CQT0, B6CQS1, H9NJ57, ref:23394, A0A1J0RET5, B6CQT1, D7SY74, B7TWE7, M5XTC6, B6CQR8, Q2I6V8, ref:23394, Q6QHU3, B6CQS7, B5KVN9, Q6QHU2, H9NJ55, 024248, B5KVP1, B6CQR7, ref:13687, ref:23394, ref:23394, H9NJ58, B6CQS2, ref:23394, A0A2H5CUG2, Q4VPK7, B6CQS9, H9NJ56, B7TWE8, Q4VPK6, B7TWE6, O22521, Q6QHU1, Q5VJR4, ref:23394, Q5VJR5, Q4VPK0, A0AUC8, B0B0L5, A0AUG9, Q4VPK5, Q5VJR2, A0AUE3, A0AUF9, B0B0L6, B6RQS3, B0B0L9, Q256S7, Q256S2, B0B0M5, Q4VPJ8, F6LWF9, Q4VPJ9, ref:23394, A0AUG8, Q5VJR3, Q4VPJ7, Q3T923, G8E012, B7TWE4, F6LWG7, E4Z8P8, A0AUF7, ref:23394, F6LWG8, ref:23394, ref:23394, Q4VPH9, F6LWG6, Q256S6, F6LWG3, B7TWE3, ref:23394, B7TWE5, ref:23394, Q5VJR1, ref:23394, D0E0C7, Q4VPI3, Q4VPI0, Q5VJQ9, Q84LA7, Q4VPI6, B6CQS4, Q941P8, Q5VJR0, A0AUH0, B6CQS3, Q256S4, Q4VPJ5, Q941P7, B9RTC1, ref:23394, Q4VPI9, Q39454, Q43549, Q08407, Q4VPJ0, G8H6R0, Q9SYV2, Q9SYV7, P43211, Q4VPI7, Q9SYW3, Q5VJQ8, Q9S7M5, Q40280, E9M219, A0AUB1, Q941P6, Q08407, H9NJ54, Q9SYV4, Q08407, Q9SYV6, Q9SYV5, Q39415, Q40280, Q941P5, Q9SYV8, Q9SYV3, P43211, F6M053, Q9SYV9, Q8L6K9, Q9FPK4, F5CEW9, P80889, A0AU76, Q96378, B6RQR9, B6RQR6, O65200, Q96377, B9RTC5, B6RQR7, Q9FPK3, A0AU75, E2GL17, B6RQS1, Q39453, P38948, B6RQS0, B6RQR8, P93105, Q9SWR4, Q9FPK2, Q96503, Q96379, A8W7B6, Q39425, Q9LEP0, Q08407, A0AU70, Q4VPJ1, C0IW10, Q39427, P38950, K4CWC4, P38949, C0IW09, Q96501, P45431, P43176, A0AU71, Q39429, P43184, P43186, Q96382, E9M220, Q96381, Q39420, Q96370, Q39428, O23754, O23752, C0IVZ2, Q96380, Q39430, Q9SCI2, C0IVZ5, Q0QLT4, P43179, F6KDF1, 024642, C0IW05, C0IW03, C0IW04, C0IVZ8, C0IW07, C0IW00, Q0QLT5, O23747, Q0QLU7, Q9ZS38, Q546U3, P15494, C0IVZ0, C0IVY9, P43183, Q96366, Q9SCH8, Q39426, O23749, Q42499, P38949, C0IVZ3, Q96371, C0IVY2, C0IVY6, C0IVY7, Q0QKW8, P43178, C0IVZ1, Q4VPJ3, Q9SYW2 and Q0QLT9.

### Method for Use in Allergy Diagnostics

A consensus allergen of the present invention may also be used in allergy diagnostics. In some embodiments, the consensus allergen is for diagnostic and/or prognostic use. In some embodiments, the consensus allergen is deposited onto a membrane, such as but not limited to a cellulose membrane. In other embodiments, the consensus allergen is deposited and/or covalently linked to a bead and/or a particle. In embodiments, the consensus allergen is used in a method for detection of an anti-allergen antibody. In some embodiments, the consensus allergen is for use in a lateral flow device, prick test, enzyme-linked immunosorbent assay (ELISA), single or multiplex beads-based epitope assay (BBEA). Accordingly, in one aspect, the present invention provides a prick test, enzyme-linked immunosorbent assay (ELISA), single or multiplex beads-based epitope assay (BBEA) or lateral flow assay capable of detecting the presence of antibodies against a range of allergens in a subject who is suspected of being allergic to one or more wild-type protein allergens in the same group, whether symptomatic or asymptomatic. In one embodiment, the assay comprises antibodies that are specific for one or more human antibodies in combination with one or more consensus allergens, such as for example, a consensus allergen with an amino acid sequence according to any one of SEQ ID NO: 1-4, or variants thereof, with an amino acid sequence that is at least 80% identical to any one of SEQ ID NOs: 1-4. In embodiments, the assay is comprised in a device. A lateral flow assay (LFA) device comprises an absorbent membrane with proteins and/or antibodies bound to different regions of the membrane. In some embodiments, anti-human antibody antibodies are bound in a first region of the membrane, i.e., are closest to the end of the membrane where the biological sample is applied. The anti-human antibody antibodies can be labelled with any detector label known in the art, including but not limited to gold, chromogenic labels, and/or fluorescent labels. In some embodiments, the consensus allergen is bound in a first or second region of the membrane, i.e., the second region is downstream of the first region. The consensus allergen can be labelled with any detector label known in the art, including but not limited to gold, chromogenic labels, and/or fluorescent labels.

Accordingly, the present invention also relates to the use of a lateral flow assay, comprising a consensus allergen of the invention. In one embodiment, the lateral flow assay is comprised in a kit, wherein said kit comprises said lateral flow assay, a sample holder for a biological sample and instructions for use. Accordingly, the present invention relates to us of the consensus allergens in allergy diagnostics, preferably, wherein the diagnostics comprises detecting reactive IgEs from serum, preferably serum from a subject suspected of being allergic to one or more protein allergens. Accordingly, in embodiments, the consensus allergen is immobilized in a sample holder, such as in one or more wells of a 12-, 24-, 48-, 96- or 384-well plate or in one or more tubes suitable as sample holder. Alternative sample holders are known to the skilled person. In further embodiments, plasma, such as mammalian plasma, such as plasma from a subject, such as a subject suspected of being allergic, is added to the sample holder(s) comprising immobilized consensus allergen(s). In further embodiments, the sample holder(s) is/are titrated with different concentrations of plasma, such as mammalian plasma, such as plasma from a subject, such as a subject suspected of being allergic. Without being bound by theory, after washing of a sample holder comprising one or more immobilized consensus allergen(s), the IgEs contained in the serum, bound to the consensus allergen, can be detected with a labelled anti-IgE antibody that will give a colorimetric, fluorometric or luminescent signal proportional to the amount of IgEs that recognize the consensus allergen(s). Accordingly, in embodiments, the consensus allergens are detected using an anti-IgE antibody, such as by colorimetric, visual, fluorometric or luminescent detection.

In embodiments, the consensus allergens of the present invention may be for use in a prick test, wherein the consensus allergens or a comprising a consensus allergen is applied subdermically to a subject, such as a patient.

Without being bound by theory, a subject which is allergic to one or more allergens, mimicked by the consensus allergen may develop an allergic reaction following application of a prick test comprising a consensus allergen of the present invention, such an allergic reaction is generally characterized by redness, itchiness, and inflammation around the application point.

In embodiments, the biological sample is collected from blood. In embodiments, the biological sample is selected from the group consisting of saliva, mucus, sputum, phlegm, nasopharyngeal secretions, blood, serum, plasma, and urine. In further embodiments, the biological sample is selected from the group consisting of a nasopharyngeal swab, a throat swab, saliva, mucus, nasopharyngeal secretions, and/or sputum. In additional embodiments, the biological sample is collected from a nasopharyngeal or a throat swab.

### Method for Providing a Consensus Allergen

The present invention also relates to a method for providing a consensus allergen, based upon input of a number of similar protein allergen sequences.

In embodiments, the consensus allergen is derived from a consensus sequence of the amino acid sequences of at least 5, 7, 10, 15, 20, 25, 30, 35, 50, 70, or at least 100 protein allergen sequences, such as at least 5, 7, 10, 15, 20, 25, 30, 35, 50, 70, or at least 100 wild-type protein allergen sequences.

### Sequence Alignment

In some embodiments, the consensus sequence of the present invention is obtained by sequence alignment of said protein allergens.

In some embodiments, the consensus allergen is obtained by:
a) selecting at least five (5) amino acid sequences of protein allergens of the present invention,
b) performing an alignment of the amino acid sequences of said protein allergens, and
c) determining the consensus sequence of said protein allergens from said alignment, wherein the selection of amino acids in the consensus sequence is based on the incidence number of the specific amino acid in each specific position in the sequence of said alignment.

In further embodiments, when two amino acids are equally represented in a position (n) in the aligned sequence, the amino acid with the highest molecular volume according to table 3, is selected as the conserved amino acid.

In further embodiments, when two or more amino acids, such as two, three, four, five, six, seven, eight, nine or ten amino acids remain equally represented in a position (n) following selection of the amino acid with the highest molecular volume according to table 3, the conserved amino acid is selected based on the physicochemical properties of amino acids, according to table 2. In embodiments the amino acids are grouped by their physicochemical properties according to:
group 1 [polar] comprising Asn, Gln, Ser and Thr,
group 2 [aliphatic] comprising Val, Ala, Leu, lie, and Met,
group 3 [basic] comprising Lys, Arg, and His,
group 4 [acidic] comprising Asp, and Glu,
group 5 [aromatic] comprising Phe, Trp, and Tyr,
group 6 comprising Pro,
group 7 comprising Gly, and
group 8 comprising Cys.

In further embodiments, the physicochemical properties are ranked according to the scoring matrix presented in table 2. Accordingly, in embodiments when two or more amino acids remain equally represented following selection of the amino acid with the highest molecular volume according to table 3, and there are two different residues that are equally represented in one position (n) of the aligned sequences, the consensus outcome of the comparison for each position is determined from table 2.

In alternative embodiments, said selection of conserved amino acids in the consensus sequence, when two amino acids are equally represented in a position (n) in the aligned sequence, the consensus outcome of the comparison for each position is determined from table 2.

In further embodiments, when two or more amino acids, such as two, three, four, five, six, seven, eight, nine, ten or more amino acids remain equally represented following selection of the amino acid according to the scoring matrix of table 2, and there are two different residues that remain equally represented in one position in the aligned sequences (n) the consensus outcome of the comparison for each position is determined by molecular volume, wherein the amino acid with the highest side chain volume is selected as the conserved amino acid.

In embodiments, polar amino acids comprise Asn, Gln, Ser, and Thr, aliphatic amino acids comprise Val, Ala, Leu, Ile, and Met, basic amino acids comprise Lys, Arg, and His, acidic amino acids comprise Asp and Glu, aromatic amino acids comprise Phe, Trp, and Tyr, while Pro, Gly, and Cys are in independent groups.

Accordingly, in embodiments, when two groups are equally present in a position in the consensus sequence the final conserved amino acid in the specific position in the consensus allergen sequence is selected according to the selection criteria defined in Table 2.

TABLE 2

| | | Outcome | | | Outcome |
|---|---|---|---|---|---|
| Residue in | Residue in | Consensus | Residue in | Residue in | Consensus |
| position n | position n | residue for | position n | position n | residue for |
| in sequence 1 | in sequence 2 | position n | in sequence 1 | in sequence 2 | position n |
| polar | aliphatic | polar | aromatic | proline | proline |
| polar | basic | basic | aromatic | glycine | glycine |
| polar | acidic | acidic | aromatic | cysteine | cysteine |
| polar | aromatic | aromatic | aromatic | gap | aromatic |
| polar | proline | proline | proline | polar | proline |
| polar | glycine | glycine | proline | aliphatic | proline |
| polar | cysteine | cysteine | proline | basic | proline |
| polar | gap | polar | proline | acidic | proline |
| aliphatic | polar | polar | proline | aromatic | proline |
| aliphatic | basic | basic | proline | glycine | proline |
| aliphatic | acidic | acidic | proline | cysteine | proline |
| aliphatic | aromatic | aromatic | proline | gap | proline |
| aliphatic | proline | proline | glycine | polar | glycine |
| aliphatic | glycine | glycine | glycine | aliphatic | glycine |
| aliphatic | cysteine | cysteine | glycine | basic | glycine |
| aliphatic | gap | aliphatic | glycine | acidic | glycine |
| basic | polar | basic | glycine | aromatic | glycine |
| basic | aliphatic | basic | glycine | proline | proline |
| basic | acidic | basic | glycine | cysteine | glycine |
| basic | aromatic | aromatic | glycine | gap | glycine |
| basic | proline | proline | cysteine | polar | cysteine |
| basic | glycine | glycine | cysteine | aliphatic | cysteine |
| basic | cysteine | cysteine | cysteine | basic | cysteine |
| basic | gap | basic | cysteine | acidic | cysteine |
| acidic | polar | acidic | cysteine | aromatic | cysteine |
| acidic | aliphatic | acidic | cysteine | proline | proline |
| acidic | basic | basic | cysteine | glycine | glycine |
| acidic | aromatic | aromatic | cysteine | gap | cysteine |
| acidic | proline | proline | gap | polar | polar |
| acidic | glycine | glycine | gap | aliphatic | aliphatic |
| acidic | cysteine | cysteine | gap | basic | basic |
| acidic | gap | acidic | gap | acidic | acidic |
| aromatic | polar | acidic | gap | aromatic | aromatic |
| aromatic | aliphatic | acidic | gap | proline | proline |
| aromatic | basic | aromatic | gap | glycine | glycine |
| aromatic | acidic | aromatic | gap | cysteine | cysteine |

Accordingly, in some embodiments, the invention relates to a method for providing a consensus allergen for use in a vaccine comprising:
a) selecting protein allergens based on their amino acid sequences,
b) performing an alignment of the amino acid sequences of said protein allergens, and
c) determining the consensus sequence of said allergens from said alignment, wherein the selection of amino acids in the consensus sequence is based on the incidence number of the specific amino acid in each specific position in the sequence of said alignment, wherein
   i. said selection of conserved amino acids in the consensus sequence, when two amino or more acids, such as two, three, four, five, six, seven, eight, nine or ten amino acids are equally represented in a position in the aligned sequence, the amino acid with the highest molecular volume according to table 3, is selected as the conserved amino acid,
   ii. when two or more amino acids, such as two, three, four, five, six, seven, eight, nine or ten amino acids remain equally represented following selection of the bulkiest amino acid, the conserved amino acid is selected based on the physicochemical properties of amino acid according to the following groups:
      group 1 [polar] comprising Asn, Gln, Ser, and Thr,
      group 2 [aliphatic] comprising Val, Ala, Leu, Ile, and Met,
      group 3 [basic] comprising Lys, Arg, and His,
      group 4 [acidic] comprising Asp and Glu,
      group 5 [aromatic] comprising Phe, Trp, and Tyr,
      group 6 comprising Pro,
      group 7 comprising Gly, and
      group 8 comprising Cys,
      wherein the conserved amino acid in the specific position is selected based on the selection criteria defined in Table 2; and
d) providing a consensus allergen sequence, based on the consensus sequence of said selected protein allergens.

In further embodiments, the invention relates to a method for providing a consensus allergen for use in a vaccine comprising:
a) selecting allergens based on their amino acid sequences,
b) performing an alignment of the amino acid sequences of said allergens, and
c) determining the consensus sequence of said allergens from said alignment, wherein the selection of amino acids in the consensus sequence is based on the incidence number of the specific amino acid in each specific position in the sequence of said alignment, wherein
   i) said selection of conserved amino acids in the consensus sequence, when two or more amino acids, such as two, three, four, five, six, seven, eight, nine, ten or more amino acids are equally represented in a position (n) in the aligned sequence, the conserved amino acid is selected based on the physicochemical properties of the amino acids according to the following groups:
      group 1 [polar] comprising Asn, Gln, Ser and Thr,
      group 2 [aliphatic] comprising Val, Ala, Leu, Ile, and Met,
      group 3 [basic] comprising Lys, Arg, and His,
      group 4 [acidic] comprising Asp, and Glu,
      group 5 [aromatic] comprising Phe, Trp, and Tyr,
      group 6 comprising Pro,
      group 7 comprising Gly, and
      group 8 comprising Cys,
      wherein the conserved amino acid in the specific position is selected based on the selection criteria defined in Table 2; and
   ii) when two or more amino acids, such as two, three, four, five, six, seven, eight, nine or ten amino acids or more amino acids remain equally represented following selection according to i) the amino acid with the highest molecular volume according to Table 3, is selected as the conserved amino acid,
d) providing a consensus allergen sequence, based on the consensus sequence of said selected allergens.

In further embodiments, when the equally represented amino acids in step ii) are Leucine and Isoleucine, Isoleucine is selected as the conserved amino acid.

TABLE 3

| | Molecular | Molar |
|---|---|---|
| | weight | volume |
| Amino acid | (g/mol) | (cm³/mol) |
| Glycine | 75.07 | 43.2 |
| Alanine | 89.09 | 60.4 |
| Valine | 117.15 | 90.8 |
| Leucine* | 131.18 | 107.5 |
| Isoleucine* | 131.18 | 107.5 |
| Phenylalanine | 165.19 | 121.2 |
| Serine | 105.09 | 60.3 |
| Threonine | 119.12 | 76.8 |
| Methionine | 149.21 | 105.3 |
| Cysteine | 121.16 | 73.3 |
| Tyrosine | 181.19 | 123.1 |
| Tryptophane | 204.23 | 143.9 |
| Aspartic acid | 132.10 | 73.8 |
| Glutamic acid | 146.12 | 85.9 |
| Asparagine | 132.12 | 78.0 |
| Glutamine | 146.15 | 93.9 |
| Lysine | 147.20 | 108.5 |
| Arginine | 175.21 | 127.3 |
| Histidine | 155.16 | 98.8 |
| Proline | 115.13 | 81.0 |

*In the case of leucine and isoleucine, which are of equal molecular weight and equal molecular volume Isoleucine is preferred.

Accordingly, the invention also relates to an isolated polypeptide comprising a consensus allergen, wherein said consensus allergen is determined with the method of the present invention.

Furthermore, the invention further relates to a nucleotide vaccine comprising a nucleic acid construct encoding a consensus allergen, wherein said consensus allergen is determined with the method of the present invention.

In some embodiments, the method of the present invention is used to generate one or more consensus allergens. In further embodiments, the method of the present invention is used to generate consensus allergens for use in a vaccine. In other embodiments, the method of the present invention is used to generate consensus allergens for use in a broad spectrum vaccine. In some embodiments, the method of the present invention is used to generate consensus allergens for use in a vaccine for the treatment of allergy. In other embodiments, the method of the present invention is used to generate consensus allergens for use in a vaccine for the use in the treatment of peach-cypress syndrome. In further embodiments, the method of the present invention is used to generate consensus allergens for use in treatment and/or amelioration of allergenic symptoms. In other embodiments, the method of the present invention is used to generate consensus allergens for use as a medicament. In further embodiments, the method of the present invention is used to generate consensus allergens for use in an mRNA or DNA vaccine. In other embodiments, the method of the present invention is used to generate consensus allergens for use in a polypeptide-based vaccine. In further embodiments, the method of the present invention is used to generate consensus allergens for use in a nucleic acid-based vaccine.

### Sequence Identity

In the present invention, protein allergens, such as wild-type protein allergens, used to generate a consensus allergen according to the current invention may have a low sequence identity over the entire length of the allergens, such as 50%, 40%, 30%, 20%, 15%, 10% or lower sequence identity. It is commonly known that allergens from different species often share a high sequence identity over short motifs and/or epitopes, while other regions are very heterogeneous, leading to cross-reactivity with the IgEs of sensitized patients. Thus, in some embodiments of the present invention the protein allergens share at least 20%, 30%, 40%, 50%, 60%, 70%, or at least 80% amino acid sequence identity over a sequence length of at least 60, 65, 70, 85, 90, 95, 100, 110, or 115 amino acids.

The term “sequence identity” as used herein describes the relatedness between two amino acid sequences or between two nucleotide sequences, i.e., a consensus allergen sequence (e.g., a sequence of the invention) and a reference sequence (such as a wild-type protein allergen sequence) based on their pairwise alignment. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “longest identity” (obtained using the -nobrief option) is used as the percent identity and is calculated as follows:

$$( Identical Residues \times 100 ) / ( Length of Alignment - Total Number of Gaps in Alignment )$$

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the DNAFULL (EMBOSS version of NCBI NUC4.4) substitution matrix. The output of Needle labelled “longest identity” (obtained using the -no brief option) is used as the percent identity and is calculated as follows:

$$( Identical Deoxyribonucleotides \times 100 ) / ( Length of Alignment - Total Number of Gaps in Alignment )$$

### Sequence Similarity

In the present invention, protein allergens used to generate a consensus allergen according to the current invention may have a low sequence identity as described above. On the other hand, the protein allergens, such as the wild-type protein allergens might share a higher degree of sequence similarity over the entire allergen sequence. Thus, in embodiments of the present invention the protein allergens at least 70%, 75%, 80%, 85%, 90%, 95%, 98%, or at least 95% sequence similarity.

The term “sequence similarity” as used herein describes the similarity between two amino acid sequences or between two nucleotide sequences, e.g., the similarity between two or more polypeptide wild type allergens based on their pairwise or multiple alignment. The sequence similarity is determined comparing the physicochemical properties of each residue such as but not limited to their molecular weight (table 3) and/or physiochemical properties, such as but not limited to charge, flexibility, side chain pKa, reactive groups, molecular volume etc. The similarity score is defined based upon a scoring matrix, defining which features are used for the similarity scoring.

### Scoring Matrices

In embodiments, the scoring used for sequence similarity is based on BLOSUM 62. BLOSUM (BLOcks SUbstitution Matrix) matrix is a substitution matrix used for sequence alignment of proteins and used to score alignments between evolutionarily divergent protein sequences. BLOSUM matrices are based on local alignments. Accordingly, in embodiments, the sequence similarity between two sequences is based upon local or global alignment, wherein the scoring matrix is a BLOSUM matrix, such as but not limited to BLOSUM90, BLOSUM80, BLOSUM62, BLOSUM50 and BLOSUM45. In general, all BLOSUM matrices are based on observed alignments from known proteins. Alternatives to the BLOSUM matrices are the PAM matrices. Accordingly, non-limiting examples of scoring matrices are PAM100, PAM120, PAM160, PAM250, PAM200, BLOSUM90, BLOSUM80, BLOSUM62, BLOSUM50, and BLOSUM45. Alternative scoring matrices are known to the skilled person, and is not limited to the herein described matrices.

### Nucleic Acid Construct

### Nucleic Acid Construct Encoding the Consensus Allergen

The present invention also relates to a nucleic acid construct comprising a nucleic acid sequence encoding a consensus allergen of the present invention. The nucleic acid construct of the present invention may be a DNA or RNA construct and may comprise modified nucleotides.

In embodiments, the nucleic acid construct of the invention comprises one or more, such as at least 1, 2, 3, 4, 5, 6, 7, 8, 9, 10, 15, or at least 20, or between 1 and 20 copies of the nucleic sequences encoding a consensus allergen of the present invention. In additional embodiments, the nucleic acid construct of the present invention comprises more than one nucleic acid encoding a consensus allergen. In additional embodiments, the nucleic acid construct of the present invention comprises more than one open reading frame encoding a consensus allergen, thereby encoding at least two different, non-identical consensus allergens.

In embodiments, the nucleic acid construct is an RNA construct. In further embodiments, the nucleic acid construct is for use in a nucleic acid vaccine. In further embodiments, the nucleic acid construct is for use in an RNA vaccine. In further embodiments, the nucleic acid construct is for use in an DNA vaccine. In further embodiments, the nucleic acid construct is for use in the production of a polypeptide vaccine. In further embodiments, the nucleic acid construct is for use in the production of an RNA vaccine. In further embodiments, the nucleic acid construct is an RNA sequence for use in an RNA vaccine.

The present invention, accordingly, in embodiments relates to an RNA vaccine encoding one or more consensus allergens of the present invention. Embodiments of the present invention relate to an RNA vaccine, encoding a consensus allergen, for use in the treatment of allergy. In further embodiments, the present invention relates to an RNA vaccine for use in the amelioration of allergic symptoms in a subject in the need thereof. The invention also relates to the use of an RNA vaccine of the present invention.

In embodiments, the present invention relates to an RNA vaccine for use in the treatment of peach-cypress syndrome. In embodiments, the present invention relates to an RNA vaccine for use in the treatment of Birch-Apple syndrome.

In further embodiments, the invention relates to an isolated DNA or RNA molecule comprising a nucleic acid sequence encoding a consensus allergen with an amino acid sequence selected from the group consisting of SEQ ID NOs 1-4 and functional homologues thereof with an amino acid sequence which is at least 80%, 85%, 90%, 95%, 97%, 98% or 99% identical to any one of SEQ ID NOs 1-4. In further embodiments, the invention relates to an isolated DNA or RNA molecule comprising a nucleic acid sequence encoding a consensus allergen with an amino acid sequence according to SEQ ID NO: 1 or functional homologues thereof with an amino acid sequence which is at least 80%, 85%, 90%, 95%, 97%, 98% or 99% identical to SEQ ID NOs 1. In further embodiments, the invention relates to an isolated DNA or RNA molecule comprising a nucleic acid sequence encoding a consensus allergen with an amino acid sequence according to SEQ ID NO: 2 or functional homologues thereof with an amino acid sequence which is at least 80%, 85%, 90%, 95%, 97%, 98% or 99% identical to SEQ ID NOs 2. In further embodiments, the invention relates to an isolated DNA or RNA molecule comprising a nucleic acid sequence encoding a consensus allergen with an amino acid sequence according to SEQ ID NO: 3 or functional homologues thereof with an amino acid sequence which is at least 80%, 85%, 90%, 95%, 97%, 98% or 99% identical to SEQ ID NOs 3. In further embodiments, the invention relates to an isolated DNA or RNA molecule comprising a nucleic acid sequence encoding a consensus allergen with an amino acid sequence according to SEQ ID NO: 4 or functional homologues thereof with an amino acid sequence which is at least 80%, 85%, 90%, 95%, 97%, 98% or 99% identical to SEQ ID NOs 4.

In further embodiments, the invention relates to an isolated DNA or RNA molecule comprising a nucleic acid sequence encoding a consensus allergen with an amino acid sequence selected from the group consisting of SEQ ID NOs 20-23 and functional homologues thereof with an amino acid sequence which is at least 80%, 85%, 90%, 95%, 97%, 98% or 99% identical to any one of SEQ ID NOs 20-23. In further embodiments, the invention relates to an isolated DNA or RNA molecule comprising a nucleic acid sequence encoding a consensus allergen with an amino acid sequence according to SEQ ID NO: 20 or functional homologues thereof with an amino acid sequence which is at least 80%, 85%, 90%, 95%, 97%, 98% or 99% identical to SEQ ID NOs 20. In further embodiments, the invention relates to an isolated DNA or RNA molecule comprising a nucleic acid sequence encoding a consensus allergen with an amino acid sequence according to SEQ ID NO: 21 or functional homologues thereof with an amino acid sequence which is at least 80%, 85%, 90%, 95%, 97%, 98% or 99% identical to SEQ ID NOs 21. In further embodiments, the invention relates to an isolated DNA or RNA molecule comprising a nucleic acid sequence encoding a consensus allergen with an amino acid sequence according to SEQ ID NO: 22 or functional homologues thereof with an amino acid sequence which is at least 80%, 85%, 90%, 95%, 97%, 98% or 99% identical to SEQ ID NOs 22. In further embodiments, the invention relates to an isolated DNA or RNA molecule comprising a nucleic acid sequence encoding a consensus allergen with an amino acid sequence according to SEQ ID NO: 23 or functional homologues thereof with an amino acid sequence which is at least 80%, 85%, 90%, 95%, 97%, 98% or 99% identical to SEQ ID NOs 23.

In further embodiments, the invention relates to an isolated DNA or RNA molecule comprising a nucleic acid sequence encoding a consensus allergen wherein the nucleic acid sequence is selected from the group consisting of nucleic acid sequences according to SEQ ID NO: 5-12, and variants thereof with nucleic acid sequences that are at least 70%, 80, 85, 90, 95, 97, 98 or 99% identical to any one of SEQ ID NOs 5-12. Preferably, the invention relates to an isolated DNA or RNA molecule comprising a nucleic acid sequence encoding a consensus allergen wherein the nucleic acid sequence is selected from the group consisting of nucleic acid sequences according to SEQ ID NO: 5, 7, 9, 11, and variants thereof with nucleic acid sequences that are at least 70%, 80, 85, 90, 95, 97, 98 or 99% identical to any one of SEQ ID NOs 5, 7, 9 or 11. In further embodiments, the invention relates to an isolated RNA molecule comprising an RNA sequence encoding a consensus allergen wherein the nucleic acid sequence is selected from the group consisting of nucleic acid sequences according to SEQ ID NO: 13-16, and variants thereof with nucleic acid sequences that are at least 70%, 80, 85, 90, 95, 97, 98 or 99% identical to any one of SEQ ID NOs 13-16. In preferred embodiments, said RNA molecule is an mRNA molecule.

Self-Amplifying mRNA or Non-Replicating mRNA Sequence.

An RNA vaccine according to the present invention comprises an RNA sequence encoding one or more consensus allergen(s). An RNA sequence of the invention is often adapted with respect to its codon usage. Accordingly, in embodiments, the RNA vaccine of the present invention comprises a codon optimized RNA sequence, which is optimized for expression in a subject, such as a mammal. Adaption of codon usage can increase translation efficacy and half-life of the RNA. In embodiments, a poly A tail comprising at least 30 adenosine residues is attached to the 3′ end of the RNA sequence to increase the half-life of the RNA. In further embodiments, the 5′ end of the RNA is capped with a modified ribonucleotide with the structure m⁷G(5′)ppp(5′)N (cap 0 structure) or a functional homolog thereof, such as but not limited to m⁷GpppNm, which can be incorporated during RNA synthesis or can be enzymatically engineered after RNA transcription by using Vaccinia Virus Capping Enzyme (VCE, consisting of mRNA triphosphatase, guanylyl-transferase and guanine-7-methyltransferase). Vaccinia Virus Capping Enzyme (VCE, consisting of mRNA triphosphatase, guanylyl-transferase and guanine-7-methyltransferase) catalyzes the construction of N7-monomethylated cap 0 structures. In general, cap 0 structure plays a crucial role in maintaining the stability and translational efficacy of the RNA vaccine. In further embodiments the 5′ cap of the RNA vaccine is modified to comprise a cap 1 structure (m⁷Gppp[m^{2′-O}]Np). The cap1 structure is generated by 5′end modification by a 2′-O-Methyltransferase. A 5′end cap1 structure which further increases translation efficacy.

In preferred embodiments, the nucleic acid construct comprises at least one self-amplifying mRNA or at least one non-replicating mRNA sequence.

RNA vaccines can be further optimised by conversion into self-replicating vaccines. In embodiments, the RNA vaccine of the present invention is a self-replicating RNA vaccine. In a self-replicating vaccine, the nucleic acid construct of the present invention includes RNA replication elements, such as but not limited to one or more replicases. In embodiments, the self-replication elements, such as one or more replicases are derived from alphaviruses. In embodiments, self-replicating RNA vaccines of the present invention comprises a replicase RNA molecule derived from semliki forest virus (SFV), sindbis virus (SIN), Venezuelan equine encephalitis virus (VEE), Ross-River virus (RRV), or other viruses belonging to the alphavirus family. In embodiments, the replicase encoding sequence is part of the nucleic acid construct. In other embodiments, the replicase encoding sequence is not part of the nucleic acid construct of the present invention. In embodiments, the nucleic acid construct further comprises a sub-genomic promoter downstream of the replicase encoding sequence. In further embodiments, the sub-genomic promoter modulates replication of the consensus allergen encoding RNA. In further embodiments, the sub-genomic promoter is followed by an artificial poly A tail comprising at least 30 adenosine residues. Replicase-based RNA vaccines have been demonstrated to induce antibody as well as cytotoxic responses at extremely low doses due to immune activation mediated by virus-derived danger signals (Ying, H. et al. (1999) Nat Med 5:823-827).

Accordingly, in embodiments, the nucleic acid construct comprises a 5′-end cap, such as cap0 or cap1, one or more coding sequence(s) (CDS), such as one or more nucleic acid sequences encoding one or more consensus allergen(s), a poly-A tail and/or a replicase encoding nucleic acid sequence. In further embodiments, the nucleic acid construct further comprises one or more elements selected from the groups consisting of 5′-end UTR, 3′-end UTR, β-globin leader sequence, cap0 and cap1.

In further embodiments, the nucleic acid construct comprises one or more modified nucleotides, such as but not limited to modified nucleotides selected from the group consisting of sugar modified nucleotides, backbone modified nucleotides, base modified nucleotides, and unnatural- and non-canonical bases.

In embodiments, the nucleic acid construct is a DNA construct or and RNA construct which comprises at least one further open reading frame encoding an RNA sequence and/or a polypeptide. In embodiments, the nucleic acid construct is a DNA construct which comprises at least one further open reading frame encoding an RNA sequence and/or a polypeptide. In embodiments, the nucleic acid construct is an RNA construct which comprises at least one further open reading frame encoding one or more polypeptides.

### Polypeptide Comprising the Consensus Allergen

The present invention also relates to a polypeptide comprising a consensus allergen of the present invention. Accordingly, in embodiments, a polypeptide of the present invention comprises a consensus allergen with an amino acid sequence selected from the group consisting of SEQ ID NOs 1-4 and functional homologues thereof with an amino acid sequence which is at least 80%, 85%, 90%, 95%, 97%, 98% or 99% identical to any one of SEQ ID NOs 1-4.

Accordingly, in embodiments, a polypeptide of the present invention comprises a consensus allergen with an amino acid sequence selected from the group consisting of SEQ ID NOs 20-23 and functional homologues thereof with an amino acid sequence which is at least 80%, 85%, 90%, 95%, 97%, 98% or at least 99% identical to any one of SEQ ID NOs 20-23.

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences, or a recombinant nucleic acid as disclosed herein, should ideally be capable of eliciting a suitable immunological response, such as a Th1 response. Preferably, a functional homologue of a consensus allergen disclosed herein should also be capable of eliciting a lower IgE response than the wild-type allergen. Preferably, a functional homologue of a consensus allergen disclosed herein should also be capable of eliciting an immunological response wherein the relative IgG1/IgG2a ratio is below 1.

In further embodiments, the polypeptide of the present invention further comprises one or more targeting and/or immunostimulatory polypeptides, such as but not limited to lysosomal targeting sequences and/or cell-penetration and/or immunostimulatory polypeptides or proteins, such as but not limited to poly-Arg, TAT, R8, DPV3, DPV6, Penetratin, pVEC, ARF(19-31), MPG and Melittin, and antibodies and fragments thereof for antigen-presenting cell targeting.

Targeting peptides can also be signal peptides that target the allergen into the endoplasmic reticulum and thereby enhance protein secretion from the cell, for example the human tissue plasminogen activator signal peptide (hTPA). In further embodiments said peptide or protein can be the lysosome-associated membrane protein (LAMP) or the 20-amino acid C-terminal tail of the lysosomal integral membrane protein-II (LIMP-II). LAMP/LIMP-II sequences may be used to direct the antigen protein to the major histocompatibility class II (MHC II) vesicular compartment of transfected professional antigen-presenting cells (APCs) thereby enhancing activation of T helper cells which increases vaccine efficacy.

### An Allergy Vaccine Composition

In embodiments, the method of the present invention is used to generate one or more consensus allergen(s). In embodiments, the method of the present invention is used to generate consensus allergens for use in a vaccine. In embodiments, the method of the present invention is used to generate consensus allergens for use in a broad spectated vaccine. In embodiments, the method of the present invention is used to generate consensus allergens for use in a vaccine for the treatment of allergy. In embodiments, the method of the present invention is used to generate consensus allergens for use in a vaccine for the use in the treatment of peach-cypress syndrome. In embodiments, the method of the present invention is used to generate consensus allergens for use in treatment and/or amelioration of allergenic symptoms. In embodiments, the method of the present invention is used to generate consensus allergens for use as a medicament. In embodiments, the method of the present invention is used to generate consensus allergens for use in an mRNA vaccine. In embodiments, the method of the present invention is used to generate consensus allergens for use in a polypeptide-based vaccine. In embodiments, the method of the present invention is used to generate consensus allergens for use in a nucleic acid-based vaccine.

In consequence, the present invention also relates to an allergy vaccine composition comprising the consensus allergen and/or a nucleic acid sequence encoding said consensus allergen of the present invention.

In that regard, in embodiments, the allergy vaccine composition comprises a consensus allergen of the present invention and/or a nucleic acid construct of the present invention. In additional embodiments, the allergy vaccine composition of the present invention also comprises additional adjuvants and/or excipients.

The nucleic acid construct comprising encoding the consensus allergen or the polypeptide comprising the consensus allergen of the present invention may be encapsulated, adsorbed to, or associated with, particulate carriers.

Suitable particulate carriers include those derived from polymethyl methacrylate polymers, as well as PLG microparticles derived from poly(lactides) and poly(lactide-co-glycolides). Other particulate systems and polymers can also be used, for example, polymers such as polylysine, polyarginine, polyomithine, spermine, spermidine, as well as conjugates of these molecules.

Accordingly, in embodiments, the nucleic acid construct and/or polypeptide is encapsulated in a nanoparticle.

Such a nanoparticle, in embodiments comprise one or more elements selected from the group consisting of lipids, proteins, peptides, dendrimers, protamines, polymers, polysaccharide, mixtures thereof and conjugates thereof. In preferred embodiments, the nanoparticle is a lipid nanoparticle. In additional embodiments, the nanoparticle or lipid nanoparticle comprises one or more permanently charged cationic lipids, such as but not limited to dioleoyl dimethylammonium chloride (DODAC), Polyethylene glycol (PEG) lipids, such as but not limited to 2-[(polyethylene glycol)-2000]-N,N-ditetradecylacetamide (PEG200-DMG), modified lipids, such as but not limited to ((4-hydroxybutyl)azanediyl)bis(hexane-6,1-diyl)bis(2-hexyldecanoate), (9-Heptadecanyl 8-{(2-hydroxyethyl)[6-oxo-6-(undecyloxy)hexyl]amino}octanoate), and/or naturally occurring lipids, such as 1,2-distearoyl-sn-glycero-3-phosphocholine and cholesterol.

In further embodiments, the composition comprising the nucleic acid construct encoding the consensus allergen or the polypeptide comprising the consensus allergen of the present invention may be a liquid, powder, granulate or lyophilizate composition. In additional embodiments, the liquid composition may be obtained by dissolving a powder, granulate or lyophilizate of a nucleic acid construct encoding the consensus allergen or the polypeptide comprising the consensus allergen described herein in a suitable solvent to provide a liquid composition. Suitable solvents may be any solvent having physiologically acceptable properties and able to dissolve the peptide combination in desired concentrations. A desired concentration may depend on the aliquot to be administered (i.e., to be administered) and the desired single dose.

An allergy vaccine composition of the invention comprises in addition to the polypeptide and/or nucleic acid construct of the invention, therapeutically inactive ingredients, such as a pharmaceutically acceptable or physiologically acceptable excipient, carrier and/or adjuvants, which are well-known to the person skilled in the art and may include, but are not limited to, solvents, emulsifiers, wetting agents, plasticizers, solubilizers (e.g. solubility enhancing agents) colouring substances, fillers, preservatives, anti-oxidants, anti-microbial agents, viscosity adjusting agents, buffering agents, pH adjusting agents, isotonicity adjusting agents, mucoadhesive substances, and the like. Examples of formulation strategies are well-known to the person skilled in the art.

Nucleic acid vaccines can also comprise adjuvants that are encoded directly in the vaccine, such as but not limited to CpG-DNA and cytokines, preferably interleukin (IL)-12 and/or IL-15.

Accordingly, a vaccine composition of the invention may contain excipients that ensure i) a suitable flowability of the nanoparticles/powder, ii) a reduction in intra-particle cohesivity, iii) a stabilization of the polypeptide or nucleic acid sequence to maintain the correct conformation, iv) a stabilization of the polypeptide or nucleic acid sequence to avoid agglomeration, and v) a suitable volume of the composition to enable handling and processing of the composition.

Amino acids may be present as a surface modifying agent to improve particle flow and reduce intra-particle cohesivity.

Amino acids may be present as a surface modifying agent to improve particle flow and reduce intra-particle cohesivity. Other suitable excipients that could be alternately used would include any phospholipid or surfactant, preferably leucine or lecithin, to include di-leucine or tri-leucine, NaCl, MgCl₂. Alternately magnesium stearate or sodium stearyl fumarate will achieve the same effect but require subsequent mechanical addition rather directly than via spray drying.

A sugar or alternative agent is present to stabilize the protein preventing agglomeration and degradation by fixing and maintaining its physicochemical conformation in an amorphous lattice. This can be achieved by a variety of sugars or sugar alcohols. Preferably disaccharides are used, and preferably trehalose. Sugars with high transition glass temperatures are preferred as this limits mobility at higher ambient moisture levels. Glucose, sucrose, lactose, dextrose, mannitol, maltitol might be considered. Amino acids and amino acid derivates, such as but not limited to leucine and/or glycine may also be included.

Accordingly, in further embodiment, the allergy vaccine composition of the invention may further comprise at least one further adjuvant.

In some embodiments, the peptide may be formulated (e.g., mixed together) with immune-modifying agents like adjuvants. The adjuvant may be any conventional adjuvant, including but not limited to oxygen-containing metal salts, e.g. aluminium hydroxide, chitosan, heat-labile enterotoxin (LT), cholera toxin (CT), cholera toxin B subunit (CTB), polymerized liposomes, mutant toxins, e.g. LTK63 and LTR72, microcapsules, interleukins (e.g. IL-1 BETA, IL-2, IL-7, IL-12, INFGAMMA), GM-CSF, MDF derivatives, CpG oligonucleotides, LPS, MPL, MPL-derivatives, phosphophazenes, Adju-Phos®, glucan, antigen formulation, liposomes, DDE, DHEA, DMPC, DMPG, DOC/Alum Complex, Freund's incomplete adjuvant, ISCOMs®, LT Oral Adjuvant, muramyl dipeptide, monophosphoryl lipid A, muramyl peptide, and phosphatidylethanolamine. Additional examples of adjuvants are described, for example, in “Vaccine Design—the subunit and adjuvant approach” (Edited by Powell, M. F. and Newman, M. J.; 1995, Pharmaceutical Biotechnology (Plenum Press, New York and London, ISBN 0-306-44867-X) entitled “Compendium of vaccine adjuvants and excipients” by Powell, M. F. and Newman M.

The vaccine or vaccine composition according to the present invention can further include an adjuvant. “Adjuvant”, according to the present invention, refers to a compound or mixture that enhances the immune response to an antigen. An adjuvant may also serve as a tissue depot that slowly releases the antigen. Adjuvants include among others complete Freund's adjuvant, incomplete Freund's adjuvant, saponin, mineral gels such as aluminium hydroxide, surface active substances such as lysolecithin, plu-ronic polyols, polyanions, peptides, Levamisol, CpG-DNA, oil or hydrocarbon emulsions, and potentially useful adjuvants such as BCG (bacille Calmette-Guerin) and Corynebacterium parvum.

Alternatively, or in addition, also immunostimulatory proteins can be provided as an adjuvant or to increase the immune response to a vaccine. Vaccination effectiveness may be enhanced by co-administration of an immunostimulatory molecule (Salgaller and Lodge, J. Surg. Oncol. (1988) 68:122), such as an immunostimulatory is for example an immunopotentiating or pro-inflammatory cytokine, lymphokine, or chemokine with the vaccine, particularly with a vector vaccine, such as a nucleic acid vaccine, such as an RNA or DNA vaccine. For example, cytokines or cytokine genes such as IL-2, IL-3, IL-12, IL-15, IL-I 8, IFN-gamma, IL-IO, TGF-beta, granulocyte-macrophage (GM)—colony stimulating factor (CSF) and other colony stimulating factors, macrophage inflammatory factor, Flt3 ligand (Lyman, Curr. Opin. Hematol., 1998, 5:192), CD40 ligand, as well as some key costimulatory molecules or their genes (e.g., B7.1, B7.2) can be used. These immunostimulatory molecules can be delivered systemically or locally as proteins or be encoded by the RNA molecule or a further nucleic acid molecule in the nuclei acid vaccine of the present invention. As immunostimulatory molecules also polycationic peptides such as poly-arginine may be employed.

In embodiments, the adjuvant is selected from the group consisting of aluminium salt-based adjuvants, emulsion adjuvants, TLR agonists, CpG-DNA and cytokines.

The invention also features a pharmaceutical composition and/or vaccine composition comprising a consensus allergen as defined herein. The pharmaceutical composition and/or vaccine composition may be a vaccine composition, e.g., a product for use in conducting immunotherapy, including but not limited to a vaccine for treating an allergic immune response to a food and/or pollen allergen.

A pharmaceutical composition and/or vaccine composition comprises in addition to the peptide combination, therapeutically inactive ingredients, such as a pharmaceutically acceptable or physiologically acceptable excipient, carrier and/or adjuvants, which are well-known to the person skilled in the art and may include, but are not limited to, solvents, emulsifiers, wetting agents, plasticizers, solubilizers (e.g. solubility enhancing agents) colouring substances, fillers, preservatives, anti-oxidants, anti-microbial agents, viscosity adjusting agents, buffering agents, pH adjusting agents, isotonicity adjusting agents, mucoadhesive substances, and the like. Examples of formulation strategies are well-known to the person skilled in the art.

In some embodiments, the pharmaceutical composition and/or vaccine composition may be formulated for parenteral administration, such as formulated for injection, e.g., subcutaneous and/or intradermal injection.

Therefore, in some embodiments, the pharmaceutical composition and/or vaccine composition may be a liquid (i.e., formulated as a liquid), including a solution, a suspension, a dispersion, and a gelled liquid. For example, a liquid pharmaceutical composition and/or vaccine composition may be formed by dissolving a powder, granulate or lyophilizate of a peptide combination described herein in a suitable solvent and then administering to a subject. Suitable solvents may be any solvent having physiologically acceptable properties and able to dissolve the peptide combination in desired concentrations. A desired concentration may depend on the aliquot to be administered (i.e., to be injected) and the desired single dose. It is emphasized that for the purposes of injection the aliquot is in the range of about 10 to 500 microliters, e.g., 50 to 300 microliters or less and a desired single dose is within range of 1 to 1000 nanomole.

Therefore, a suitable solvent should be able to dissolve consensus allergen to achieve a final concentration of about 1 to 1000 μM for the consensus allergen. Typically, the solvent is an aqueous solution, optionally mixed with other solvents. Thus, a solvent may comprise at least 60% w/w of water, e.g. at least 65% w/w, 70% w/w, 75% w/w, 80% w/w, 85% w/w, 90% w/w or 95% w/w, 99% w/w of water, such as distilled water, such as sterile water. In some embodiments, the solvent is sterile distilled water, e.g., water for injection. An aqueous solution may comprise other solvents than water, for example DMSO (dimethylsulfoxide), glycerol, ethanol, acetonitrile, vegetable, or synthetic oils. The pH of the aqueous phase of the solvent may be in a physiological acceptable range, typically in the range of 3 to 9, such as in the range of pH 3 to 8, such as in the range of pH 4 to 8, such as in the range of 5 to 8, such as in the range of 6 to 8. Thus, the liquid formulation may comprise a pH controlling agent or buffering agent (e.g., citrate buffer, phosphate buffer, acetate buffer), optionally the pH may be adjusted with dilutions of strong base (e.g. sodium hydroxide or the like) and/or dilutions of strong acids (e.g. hydrochloric acid).

Typically, the liquid formulation is isotonic, and optionally sterile. Therefore, in some embodiments, the formulation comprises saline, such as isotonic saline. The liquid may contain additional excipients, such as another solvent, a solubilizing enhancing agent (e.g., polyoxyethylene (20) sorbitan monolaurate (Tween® 20), ionic and non-ionic emulsifiers (e.g. poloxamers (Kolliphor®)), a dispersant, a thickener, a preservative, an anti-microbial agent, and/or an antioxidant. Non-limiting illustrative examples of solvents include water, saline, DMSO, glycerol, ethanol, acetonitrile, vegetable, or synthetic oils.

Some poly-peptides are known to be prone to oxidation, especially the nsLTP proteins comprising a number of Cys residues, or being unstable when exposed to water for a long period. Therefore, to achieve storage stable compositions, a pharmaceutical composition and/or vaccine composition may be formulated to contain only a limited amount of water or aqueous solution, e.g., containing less than 10% w/w of water or aqueous solution, such as less than 9, 8, 7, 6, 5, 4, 3, 2, 1, 0.5% w/w of water or aqueous solution. Examples of pharmaceutical composition and/or vaccine compositions with limited levels of water may include granulates, powders, for example lyophilizates, i.e., freeze-dried powders. Typically, the freeze-dried composition may be dissolved before use, for example dissolved in an aqueous, optionally sterile, solution, for example a solution having a pH in the range of 3-9, such as pH in the range of 3 to 8, such as pH in the range of 4 to 8. A lyophilizate may contain additional ingredients, e.g., bulking agents and lyoprotectants (e.g., sucrose, lactose, trehalose, mannose, mannitol, sorbitol, glucose, raffinose, glycine, histidine or mixtures thereof), buffering agents (e.g., sodium citrate, sodium phosphate, disodium phosphate, sodium hydroxide, Tris base, Tris acetate, Tris HCl or mixtures thereof), antioxidants, antimicrobial agents, solubilizers (e.g. polyoxyethylene (20) sorbitan monolaurate (Tween® 20)).

A freeze-dried composition may also be formulated into a solid dosage form that is administered for example by the oral route such as by oral mucosa. Thus, in some embodiments, the pharmaceutical composition and/or vaccine composition may be formulated for oral administration, for example for sublingual administration. Therefore, the pharmaceutical composition and/or vaccine composition may be a solid dosage form, such as a freeze-dried solid dosage form, typically a tablet, a capsule or sachet, which optionally may be formulated for fast disintegration. Pharmaceutical formulations and delivery systems appropriate for the compositions, methods and uses of the invention are known in the art (see, e.g., Remington: The Science and Practice of Pharmacy (2003) 20th ed., Mack Publishing Co., Easton, PA; Remington's Pharmaceutical Sciences (1990) 18th ed., Mack Publishing Co., Easton, PA; The Merck Index (1996) 12th ed., Merck Publishing Group, Whitehouse, NJ; Pharmaceutical Principles of Solid Dosage Forms (1993), Technonic Publishing Co., Inc., Lancaster, Pa.; Ansel ad Soklosa, Pharmaceutical Calculations (2001) 11th ed., Lippincott Williams & Wilkins, Baltimore, MD; and Poznansky et al., Drug Delivery Systems (1980), R. L. Juliano, ed., Oxford, N.Y., pp. 253-315).

As mentioned, pharmaceutical composition and/or vaccine compositions can be formulated to be compatible with a particular route of administration, such as by intradermal or by sublingual administration. Thus, pharmaceutical composition and/or vaccine compositions may include carriers, diluents, or excipients suitable for administration by various routes. Exemplary routes of administration for contact or in vivo delivery for which a composition can optionally include inhalation, intranasal, oral, buccal, sublingual, subcutaneous, intradermal, epicutaneous, rectal, transdermal, or intralymphatic administration routes.

For oral, buccal, or sublingual administration, a composition may take the form of, for example, tablets or capsules, optionally formulated as fast-disintegrating tablets/capsules or slow-release tablets/capsules. In some embodiments, the tablet is freeze-dried, optionally a fast-disintegrating tablet or capsule suitable for being administered under the tongue.

The pharmaceutical composition and/or vaccine composition may also be formulated into a “unit dosage form”, which used herein refers to physically discrete units, wherein each unit contains a predetermined quantity of a peptide or peptide combination, optionally in association with a pharmaceutical carrier (excipient, diluent, vehicle, or filling agent) which, when administered in one or more doses, may produce a desired effect. Unit dosage forms also include, for example, ampules and vials, which may include a composition in a freeze-dried or lyophilized state (a lyophilizate) or a sterile liquid carrier, for example that can be added prior to administration or delivery in vivo. Unit dosage forms additionally include, for example, ampules and vials with liquid compositions disposed therein. A unit dose form may be for single, sequential, or simultaneous administration.

Peptides may be prone to degradation when exposed to oxygen, for example when exposed to air or solvents containing air. Therefore, in some embodiments, the pharmaceutical composition and/or vaccine composition comprises an inert gas, such as but not limited to argon or nitrogen.

Another aspect of the invention relates to a kit comprising a compartment and instructions, wherein the compartment comprises a pharmaceutical composition and/or vaccine composition as described herein for single, sequential, or simultaneous administration, and wherein the instructions are for use in treating food and/or pollen allergy, such as cypress and/or peach and related allergies. A kit may further comprise packaging material comprising corrugated fiber, glass, plastic, foil, ampules, vials, blister pack, preloaded syringes, or tubes, optionally that maintain sterility of the components. A kit may further comprise labels or inserts comprising printed matter or computer readable medium optionally including identifying components, dose amounts, clinical pharmacology, and instructions for the clinician or for a subject using one or more of the kit components, prophylactic or therapeutic benefits, adverse side effects or manufacturer information.

In embodiments, the kit additionally comprises a container comprising a solvent for dissolving the composition before use. Examples of suitable solvents are described supra. Optionally, the kit may also comprise a device for use in parenteral injection, for example for injecting the composition (e.g., dissolved composition) to a subcutaneous or intradermal tissue. A device may be any suitable device for that purpose, such as a needle or microneedle adapted for intradermal or subcutaneous delivery of the composition.

### Allergy Vaccine

### Polypeptide Vaccine

Consensus allergens are designed sequences representative of a collection of similar allergens and, consequently, can be used to treat multiple related allergies simultaneously.

In the current context, the term “vaccine” is used as a preparation of immunogenic material (e.g., protein or nucleic acid; vaccine) capable of stimulating (eliciting) an immune response when administered to a subject, to treat a disease, condition, or pathology, or to prevent a disease, condition. Vaccines may elicit a prophylactic (preventative) immune response in the subject; they may also elicit a therapeutic response immune response in a subject. In embodiments, the vaccine is for use as a therapeutic to be administered to a subject with allergy. As mentioned above, methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous, or intramuscular.

Vaccines may also be administered with an adjuvant to boost the immune response as described herein. Accordingly, the present invention also relates to a vaccine which comprises a polypeptide comprising the consensus allergen of the invention.

In embodiments a polypeptide vaccine of the present invention wherein comprises more than one polypeptide each comprising a different consensus allergen, such as more than 2, 3, 4, 5, 6, 7, 8, 9, 10, 11, 12, 13, 14, 15, 16, 17, 18, 19 or more than 20 polypeptides each comprising a different consensus allergen. In that regard, a vaccine of the present invention may comprise more than two different consensus allergens, such as but not limited to more than 3, 4, 5, 6, 7, 8, 9, 10, 11, 12, 13, 14, 15, 16, 17, 18, 19 or more than 20 different consensus allergens.

### Derivatives

A consensus allergen as defined herein may be modified to contain “non-natural” modifications. Such peptides are referred to as variants herein and more specifically they are referred to as derivative peptides or derivatives.

The term “derivative” refers to a chemically modified form of a peptide disclosed herein. Typically, a derivative is formed by reacting a functional side group of an amino acid (e.g., amino, sulfhydryl or carboxy-group) with another molecule to form a covalent or non-covalent attachment of any type of molecule (naturally occurring or designed), such as a sugar moiety. Specific examples of derivatives of a peptide include glycosylation, acylation (e.g. acetylation), phosphorylation, amidation, formylation, ubiquitination and derivatization by protecting/blocking groups and any of numerous chemical modifications.

Additional specific non-limiting examples are tagged peptides, fusion peptides, chimeric peptides including peptides having one or more non-amino acyl groups (q.v., sugar, lipid, etc.) covalently linked to the peptide.

Typically, a derivative comprises one or more modifications, for example selected from any of: (a) N-terminal acylation (e.g., acetylation or formylation); (b) C-terminal amidation (e.g., reaction with ammonia or an amine); (c) one or more hydrogens on the side chain amines of arginine and/or lysine replaced with a methylene group; (d) glycosylation and/or (e) phosphorylation.

### Fusion Products

In embodiments, the consensus allergen may be comprised in a fusion (chimeric) poly-peptide, optionally containing an amino acid sequence having one or more additional molecules and/or poly-peptides covalently attached to the consensus allergen amino acid sequence.

Another embodiment relates derivatives wherein a second heterologous sequence, i.e., a heterologous functional domain, is attached to a consensus allergen disclosed herein, by covalent or non-covalent binding, which confers a distinct or complementary function to a consensus allergen disclosed herein. Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral), a metal (gold, silver), or a radioisotope.

Linkers, such as amino acid or peptidomimetic sequences, may be inserted between the peptide sequence and the addition (e.g., heterologous functional domain) so that the two entities maintain, at least in part, a distinct function or activity. Linkers may have one or more properties that may include a flexible conformation, an inability to form an ordered secondary structure or a hydrophobic or charged character, which could promote or interact with either domain. Amino acids typically found in flexible protein regions include Gly, Asn and Ser. Other near neutral amino acids, such as Thr and Ala, may also be used in the linker sequence.

In additional aspects of the invention, combinations of consensus allergens are not provided as individual peptides, but the peptides may be fused together or to a carrier molecule to form an isolated molecule. For example, the consensus allergens may be fused to the N- and C-terminus of a surface polypeptide of a virus, e.g., a virus of the hepadnaviridae family as disclosed in international patent application WO12168487A1.

A consensus allergen may share the same functionality such as a similar immunological response as a protein allergen, such as a wild-type protein allergen as defined herein, or may have an improved functionality, such as an improved immunological response profile, or it may have a function comparable to a range of different protein allergens. In that regard wild-type protein allergens, often has a specific function in the species from which they originate, such as germination (nsLTPs), transport (albumins), or defence (PR-10). As such a consensus allergen of the present invention may have a reduced physiological function compared to the wild-type protein allergen in the origin species of the wild-type protein allergens and may be inactive. Thus, the “same functionality” in the present invention relates to the function of the consensus protein allergen compared to the protein allergen in a host, which upon exposure to the consensus protein allergen and/or protein allergen has a physiological response to the exposure of said a consensus protein allergen and/or protein allergen.

Furthermore, a consensus allergen may comprise one or more of the same T cell or B cell epitopes as the protein allergens. This may be determined by the ability of the variant peptide to induce or stimulate in-vitro T cell proliferation using cultured PBMCs (peripheral blood monocytes) compared to the parent peptide as defined herein, optionally using same test conditions, or by the ability of the variant peptide to induce or stimulate production of cytokines, (e.g., cytokines, IL-5, IL-13 and/or IL-10) from T cells (obtained from cultured PBMC's) compared to the parent peptide.

### Salts of Polypeptides Comprising the Consensus Allergen

Peptides are typically provided in the form of a salt, for example as a pharmaceutically acceptable and/or a physiologically acceptable salt. For example, the salt may be an acid addition salt with an inorganic acid, an acid addition salt with an organic acid, a salt with a basic inorganic acid, a salt with a basic organic acid, a salt with an acidic or basic amino acid or a mixture thereof. Typical examples of an acid addition salts with an inorganic acid are selected from any of the salts with hydrochloric acid, hydrobromic acid, nitric acid, sulfuric acid, phosphoric acid, or the like. An acid salt with an organic acid may be selected from any of the salts with formic acid, acetic acid, trifluoroacetic acid, fumaric acid, oxalic acid, tartaric acid, maleic acid, succinic acid, malic acid, methanesulfonic acid, benzenesulfonic acid, p-toluenesulfonic acid, or the like. Salts with an inorganic base may be selected from a salt of an alkali metal salts such as sodium salts and potassium salts; alkali earth metal salts such as calcium salts and magnesium salts; and aluminum salts and ammonium salts. Salts with a basic organic base may be selected from any salt with trimethylamine, triethylamine, pyridine, picoline, ethanolamine, diethanolamine, triethanolamine, dicyclohexylamine, N,N-dibenzylethylenediamine, caffeine, piperidine, and pyridine. Salts with a basic amino acid may be selected from any salt with arginine, lysine, omithine, or the like. Salts with an acidic amino acid may be selected from any salt with aspartic acid, glutamic acid, or the like.

In further embodiments of the invention a salt, such as a pharmaceutically acceptable salt, is an acetate salt. Accordingly, in embodiments, the isolated polypeptide comprising a consensus allergen of. the present invention is provided as a pharmaceutically acceptable salt.

mRNA Vaccine

Nucleic acid vaccines may be administered directly into the cells of a subject via mRNA injection. Thus, in embodiments, an RNA vaccine of the present invention comprising a nucleic acid sequence encoding a consensus allergen of the invention, is for intracellular delivery, such as but not limited to by mRNA injection into a cell, such as but not limited to injection into an antigen presenting cell (APC), such as a dendritic cell. Without being bound by theory, a benefit of direct injection of the RNA vaccine is that the consensus allergen of the present invention will be expressed directly in the APC cell, such that the cell expresses the polypeptide comprising a consensus allergen. Upon expression, the polypeptide comprising the consensus allergen, may be translocated to the lysosome for degradation, whereafter epitopes of the allergen is presented on surface of the APCs. Accordingly, the mRNA-encoded consensus allergens are never fully present in the extracellular environment and cannot induce an IgE-mediated or IgE independent allergic response to the same extent as a polypeptide-based vaccine. This means that RNA vaccine encoding a polypeptide comprising a consensus allergen can be used to induce a neutralizing IgG response with lesser risk of adverse allergic reactions, solving the main drawbacks of conventional allergy immunotherapy.

Accordingly, the present invention relates to an RNA vaccine, such as but not limited to an mRNA vaccine, encoding a poly-peptide comprising the consensus allergen of the present invention. Accordingly, the invention also relates to a therapeutic composition comprising an isolated RNA or DNA according to the present invention, and the use of such a therapeutic composition as a medicament.

### Hypoallergenic Response

As used herein, the term “hypoallergenic” refers to the ability of a peptide, polypeptide or protein derived from an allergen with allergenic properties to induce the induction of T cells specific for said allergen and exhibiting reduced or no allergic reactions when administered to an individual.

In that regard, in embodiments, a vaccine of the present invention is hypoallergenic. In the present invention the term “hypoallergenic” in relation to the consensus allergen is characterized by the consensus allergen having a reduced IgE reactivity but preserved T cell epitopes to reduce IgE-mediated side effects.

The immunological response to a vaccine comprising a consensus allergen of the present invention, may be addressed according to Examples 4-6, and examples 7-8 as described herein.

RNA vaccines are rendered hypoallergenic by targeting the resulting protein into the ubiquitination pathway of the cell, where the respective protein is degraded into hypoallergenic peptides. This is achieved by fusing the sequence encoding ubiquitin to the 5′ end of the allergen encoding RNA. Ubiquitination efficacy can be enhanced by mutating amino acid residue 76 from glycine to alanine (G76->A76). Ubiquitination efficacy can be further enhanced by mutating the first amino acid of the allergen (methionine) to a destabilizing amino acid (Arginine) (M77->R77). Alternatively, ubiquitination of the resulting gene product can be achieved by adding a carboxyterminal destabilizing sequence known as PEST sequence.

Accordingly in embodiments of the present invention a hypoallergenic consensus allergen encoded by an RNA sequence exhibits an IgE reactivity which is at least 10%, preferably at least 20%, more preferably at least 30%, in particular at least 50%, lower than the IgE reactivity of one or more of the protein allergens.

Hypoallergenicity of RNA vaccines can be routinely tested by translating the RNA in vitro in a rabbit reticulocyte lysate system. The resulting gene product will be analysed by IgE western blots using pools of appropriate patients' sera. Reduction of IgE binding capacity of the respective hypoallergenic consensus allergen will be assessed compared to the IgE binding capacity of the protein allergens, such as wild-type protein allergens, translated in said reticulocyte lysate system.

This was also shown in example 7, where the mRNA-based vaccine produced an IgG1/IgG2a ratio was <1, which is indicative of a Th1 response, which does not trigger an IgE response to the same extend as a higher IgG1/IgG2a ratio normally would. This was also verified, by measuring the CA1 specific IgE levels produced in immunized mice, where the mice treated with the protein based vaccine had a higher IgE response than the mRNA treated mice.

Accordingly, in embodiments, an mRNA vaccine as disclosed herein results in a lower IgE response relative to a protein-based vaccine. In additional embodiments, an mRNA vaccine comprising a nucleic acid sequence encoding a consensus allergen as disclosed herein, induces a lower IgE response upon immunization, than a protein based consensus allergen vaccine disclosed herein, when said consensus allergen comprises an amino acid sequence selected from the group consisting of SEQ ID NOs 1, 2, 3, 4, 20, 21, 22 and 23 and functional homologues thereof with an amino acid sequence which is at least 80%, such as at least 85%, 90%, 95%, 97%, 98%, or such as at least 99% identical to any one of SEQ ID NOs 1, 2, 3, 4, 20, 21, 22 and 23.

IN embodiments, the mRNA vaccine as disclosed herein elicits an IgG1 and IgG2a response following administration, wherein the IgG1/IgG2a ratio is <1, such as between 0.01-0.99, or such as less than 0.1, 0.2, 0.3, 0.4, 0.5, 0.6, 0.7, 0.8, 0.9 or such as less than 0.99.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent. In certain embodiments, the subject animal is a mammal. The mammal may be an adult cow, a calf, in particular a juvenile calf, a rat, rabbit, pig, mouse, preferably a human, and the method comprises administering a dose of any one or more of the vaccines provided herein to the subject.

### Peach-Cypress Syndrome Vaccine

The present invention also relates to a vaccine for alleviating a Pollen Food Allergy Syndrome such as peach-cypress syndrome. In that regard, in embodiments the consensus allergen is derived from the consensus sequence of non-specific Lipid Transfer Proteins (nsLTP) such as described herein. Exemplary consensus allergens derived from nsLTP proteins are described by SEQ ID NOs: 1-4, which are generated using the method of the present invention as described in example 1-4.

In embodiments, the vaccine of the present invention comprises a composition comprising a consensus allergen with an amino acid sequence selected from the group consisting of SEQ ID NOs 1-4 and functional homologues thereof with an amino acid sequence which is at least 80%, 85%, 90%, 95%, 97%, 98%, or 99% identical to any one of SEQ ID NOs 1-4.

In embodiments, the vaccine according to the present invention comprises a composition comprising a consensus allergen with an amino acid sequence according to SEQ ID NO 1 and/or a homologue thereof with an amino acid sequence which is at least 80%, 85%, 90%, 95%, 97%, 98% or 99% identical to SEQ ID NO 1. In embodiments, the vaccine of the present invention comprises a composition comprising a consensus allergen with an amino acid sequence according to SEQ ID NO 2 and/or a homologue thereof with an amino acid sequence which is at least 80%, 85%, 90%, 95%, 97%, 98% or 99% identical to SEQ ID NO 2. In embodiments, the vaccine of the present invention comprises a composition comprising a consensus allergen with an amino acid sequence according to SEQ ID NO 3 and/or a homologue thereof with an amino acid sequence which is at least 80%, 85%, 90%, 95%, 97%, 98%, or 99% identical to SEQ ID NO 3. In embodiments, the vaccine of the present invention comprises a composition comprising a consensus allergen with an amino acid sequence according to SEQ ID NO 4 and/or a homologue thereof with an amino acid sequence which is at least 80%, 85%, 90%, 95%, 97%, 98%, or 99% identical to SEQ ID NO 4.

Accordingly, a vaccine according to the present disclosure comprises a polypeptide or a nuclei acid encoding said polypeptide, wherein the polypeptide comprises a consensus allergen with an amino acid sequence selected from the group consisting of SEQ ID NOs 1-4 and functional homologues thereof with an amino acid sequence which is at least 80%, 85%, 90%, 95%, 97%, 98%, or 99% identical to any one of SEQ ID NOs 1-4, in the treatment Peach-cypress syndrome and/or amelioration of symptoms thereof.

### Birch-Apple Syndrome Vaccine

The present invention also relates to a vaccine for alleviating a Pollen Food Allergy Syndrome such as Birch-Apple syndrome. In that regard, in embodiments the consensus allergen is derived from the consensus sequence of Pathogenic Related proteins family 10 (PR-10) such as described herein.

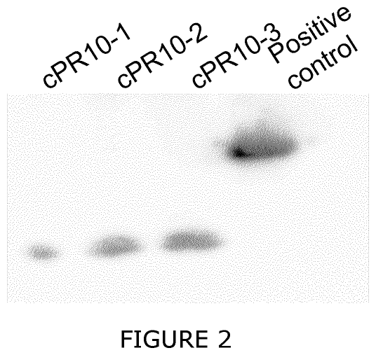

Exemplary consensus allergens derived from PR-10 proteins are described by SEQ ID NOs: 20-23, which are generated using the method of the present invention as described in example 1, 2 and 7 and FIG. 2.

In embodiments, the vaccine of the present invention comprises a composition comprising a consensus allergen with an amino acid sequence selected from the group consisting of SEQ ID NOs 20-23 and functional homologues thereof with an amino acid sequence which is at least 80%, 85%, 90%, 95%, 97%, 98%, or 99% identical to any one of SEQ ID NOs 20-23.

In embodiments, the vaccine according to the present invention comprises a composition comprising a consensus allergen with an amino acid sequence according to SEQ ID NO 20 and/or a homologue thereof with an amino acid sequence which is at least 80%, 85%, 90%, 95%, 97%, 98% or 99% identical to SEQ ID NO 20. In embodiments, the vaccine of the present invention comprises a composition comprising a consensus allergen with an amino acid sequence according to SEQ ID NO 21 and/or a homologue thereof with an amino acid sequence which is at least 80%, 85%, 90%, 95%, 97%, 98% or 99% identical to SEQ ID NO 21. In embodiments, the vaccine of the present invention comprises a composition comprising a consensus allergen with an amino acid sequence according to SEQ ID NO 22 and/or a homologue thereof with an amino acid sequence which is at least 80%, 85%, 90%, 95%, 97%, 98%, or 99% identical to SEQ ID NO 22. In embodiments, the vaccine of the present invention comprises a composition comprising a consensus allergen with an amino acid sequence according to SEQ ID NO 23 and/or a homologue thereof with an amino acid sequence which is at least 80%, 85%, 90%, 95%, 97%, 98%, or 99% identical to SEQ ID NO 23.

Accordingly, a vaccine according to the present disclosure comprises a polypeptide or a nuclei acid encoding said polypeptide, wherein the polypeptide comprises a consensus allergen with an amino acid sequence selected from the group consisting of SEQ ID NOs 20-23 and functional homologues thereof with an amino acid sequence which is at least 80%, 85%, 90%, 95%, 97%, 98%, or 99% identical to any one of SEQ ID NOs 20-23, in the treatment Birch-Apple Syndrome and/or amelioration of symptoms thereof.

### Administration of the Allergy Vaccine

The vaccine of the present invention may be administered in various ways, depending on the nature of the vaccine. One way, for instance, to administer the nucleic acid vaccine of the present invention is to transfer the nucleic acid vaccine directly into a body (e.g., intramuscular, intradermal, intravenous, intranasal etc.). Alternatively, it is possible to prepare cells ex vivo comprising nucleic acid constructs of the present invention such as an RNA sequence to be expressed. In example, epidermal cells may be transfected with the nucleic acid vaccine in vitro and then administered (transplanted) to a subject.

The cells can be transfected by exogenous or heterologous RNA when such RNA has been introduced inside the cell. The RNA can be introduced into the cells by pulsing, i.e., incubating the cells with the RNA molecules of the invention. Alternatively, the RNA can be introduced in vivo by lipofection, as naked RNA, or with other transfection facilitating agents (peptides, polymers, etc.), such as but not limited to encapsulation into nanoparticles. Synthetic cationic lipids can be used to prepare liposomes for in vivo transfection. Useful lipid compounds and compositions for transfer of nucleic acids are, e.g., DODC, DOPE, CHOL, DMEDA, DDAB, DODAC, DOTAP and DOTMA.

Other molecules are also useful for facilitating transfection of a nucleic acid in vivo, such as cationic oligopeptides (e.g., WO 95/21931), peptides derived from DNA binding proteins (e.g. WO96/25508), or cationic polymers (e.g. WO 95/21931).

Further examples of suitable delivery systems may e.g., be based upon Pfizer/BioNTech vaccine Comirnaty (BNT162b2) vaccine. Particularly, a vaccine disclosed herein may be formulated as a lipid nano particle. Such a lipid nano-particle may comprise one or more lipid components. In example, the delivery of the mRNA vaccine may e.g., be obtained by incorporation of the mRNA encoding the consensus allergen into a lipid nanoparticle. Such lipid nano particle may e.g., comprise one or more lipids selected from the group consisting of the ionizable lipid ALC-0315, the PEGylated lipid ALC-0159 lipid and DMG-PEG. In embodiments, the lipid nano-particle comprises ALC-0315 and DMG-PEG.

Also, polyethylenimine and its derivatives, polylactide-polyglycolide, and chitosan may be used. Alternatively, nucleic acid sequences, preferably, RNA molecules can be introduced into the desired host cells by methods known in the art, e.g. electropo-ration, microinjection, cell fusion, DEAE dextran, calcium phosphate precipitation, or use of biolistic transfection, see e.g. Tang et al., Nature (1992) 356: 152-154.

According to other embodiments of the present invention the vaccine is adapted for intramuscular, intradermal, intravenous, transdermal, topical, sublingual, subcutaneous, oral, nasal, ophthalmic, and/or biolistic administration.

The compositions described herein may be dosed in a dosage regimen usually applied in the field of allergy immunotherapy, such as gene or peptide allergy immunotherapy. For example, compositions may be administered as a single dose (e.g., one injection) with daily, weekly, bi-weekly, monthly, quarterly intervals over a period of at least 2-6 months or even longer until a more persistent effect is achieved, annually, bi-annually. The term “persistent effect” means that one or more clinically relevant symptoms of the immune response is reduced in the subject when exposed to an allergen of a food and/or pollen allergen compared to before the subject is administered the first dose. A persistent effect may be evaluated at least two months after the subject has stopped treatment, such as after at least three, four, five, six, nine or twelve months.

It is also envisaged that the treatment is initiated by an up-dosing phase with the vaccine of the present invention being administered in increasing doses within one day or with daily, weekly or bi-weekly intervals until the target maintenance dose is achieved.

As shown in Example 7, the protein-based vaccine disclosed herein may require multiple administrations to elicit an immunological response, while an mRNA-based vaccine may require a single administration to obtain an initial immunological response, which may be boosted by repeated administration. Accordingly, the vaccine disclosed herein may be administered once or repeatedly, such as 1, 2, 3, 4, 5, 6, 7, 8, 9 or 10 times. In addition, a single administration may be followed up days, weeks or months form the initial administration as a booster vaccine.

Alternatively, the vaccine disclosed herein may be administered as a supplement to ongoing vaccination as a supplement to a different vaccination.

In embodiments, a vaccine as disclosed herein may be administered via. subcutaneous administration. In additional embodiments, the vaccine disclosed herein may be administered in an amount in the range of 0.1-1000 ug pr. dose, such as 1-500 ug pr. dose, preferably, about 5-100 ug pr dose, such as about 5, 6, 9, 10, 12, 15, 20, 24, 25, 30, 35, 40, 45, 50, 55, 60, 65, 70, 75, 80, 85, 90, 95 or such as 100 ug pr. dose. In example 7, naïve mice were administered with 9, 3 or 0.6 ug mRNA vaccine or 24, 12 or 6 ug protein vaccine.

The subject being administered a vaccine as described herein may optionally also be administered another therapeutic agent used for treating an immune response against an allergen. Furthermore, the subject being administered a polypeptide vaccine as described herein may optionally also be administered the nucleic acid vaccine of the present invention.

In that regard, the first dosing to a patient, of the polypeptide vaccine of the present invention may be a tolerability dosing, whilst when the tolerability of the polypeptide vaccine is established, the nucleic acid vaccine, preferably, mRNA vaccine, is administered to the subject, to obtain a long-term effect of the vaccine.

In embodiments, a vaccine of the present invention is for use as a medicament. Accordingly, in embodiments a vaccine of the present invention is for use in the treatment of allergy such as but not limited to use in the treatment of peach-cypress syndrome.

### Method of Allergy Treatment

Compositions (e.g., composition comprising a polypeptide vaccine and/or a nucleic acid vaccine of the present invention) described herein may be used for the treatment of a negative immune response or allergy to an allergen, preferably a food-allergy allergen, in a subject in need thereof. Allergy to an allergen may be clinically presented in the subject as atopic dermatitis, urticaria, contact dermatitis, allergic conjunctivitis, allergic rhinitis, allergic asthma and/or anaphylaxis. Therefore, in some aspects of the present invention, the method comprises decreasing, reducing, suppressing, or inhibiting atopic dermatitis, urticaria, contact dermatitis, allergic conjunctivitis, allergic rhinitis, allergic asthma, and/or anaphylaxis.

Accordingly, the present invention also relates to methods for providing an allergy treatment.

In that regard, in embodiments, the vaccine of the present invention is for use in the treatment of an immune response. Furthermore, the present invention also relates to the use of a vaccine according to the present disclosure for the manufacture of a vaccine for treating, ameliorating and/or preventing allergy for the manufacture of a vaccine for treating, ameliorating and/or preventing allergy.

In addition, the present invention also relates to the use of a vaccine according to the present disclosure in the manufacture of a protective and/or therapeutic vaccine for hyposensitizing an individual to an allergen.

The phrase “treatment of an immune response” or “treating an immune response” may encompass preventing, relieving, alleviating, reducing, inhibiting, decreasing, or suppressing an immune response, for example an allergic immune response, such as an immune response against a pollen food associated syndrome related allergen. The treatment of an immune response may also include the decrease, inhibition, suppression, or reduction of a T cell response, which may include, but is not limited to, a Th2 cell response or a memory T cell response. Furthermore, the treatment of an immune response described herein may also comprise inducing, promoting, increasing, or enhancing proliferation of regulatory T cells while optionally decreasing, reducing, inhibiting, suppressing or reducing production of pro-inflammatory lymphokines/cytokines.

Therefore, in the present context, the invention relates to a method for relieving an immune response to an allergen, preferably a food borne allergen, such as but not limited to nsLTPs, in a subject in need thereof, comprising administering to said subject a therapeutically effective amount of a composition described herein (e.g., a polypeptide or nucleic acid vaccine as described herein).

In other aspects, the administration of a composition described herein may induce immunological tolerance against the allergen(s), preferably a pollen and/or food associated allergen, such as but not limited to nsLTPs. In other aspects the administration of a composition described herein may induce immunological tolerance against the allergens related to the birch-apple syndrome, such as but not limited to members of the Pathogenic Related proteins family 10 (PR-10).

Thus, compositions disclosed herein may produce a therapeutic or beneficial effect, which optionally may be objectively or subjectively measurable. A therapeutic or beneficial effect can, but need not, be complete ablation of all or any immune response, or one or more symptoms caused by or associated with an allergen.

Thus, a satisfactory clinical result is achieved when there is an incremental improvement or a partial reduction in an immune response or one or more symptoms caused by or associated with an allergen, or there is an inhibition, decrease, reduction, suppression, prevention, limit or control of worsening or progression of an immune response or one or more symptoms caused by or associated with an allergen over a short or long duration (hours, days, weeks, months, etc.).

Therefore, in still other aspects, the subject's administration of a therapeutically effective amount of a vaccine composition described herein may relieve one or more symptoms of the immune response. For example, the method may comprise relieving one or more symptoms associated with allergic rhinitis, allergic conjunctivitis, allergic asthma and/or allergic eczema (e.g., atopic dermatitis).

In some embodiments, the one or more symptoms may be associated with allergic rhinitis. For example, the method may comprise reducing one or more of the following symptoms: intensity of itchy nose; number of sneezes within a given period (e.g., daily, weekly, monthly); intensity of blocked nose (e.g., congestion); amount of nasal secretions; eosinophilic count in nasal secretions; specific IgE antibody level (titer) in nasal secretions or in serum; and basophil histamine release of blood.

In other embodiments, the one or more symptoms may be associated with allergic conjunctivitis. For example, the method may comprise reducing one or more of the following symptoms: intensity of itchy eyes, redness in the white of the eyes and/or watery eyes; eosinophilic count in conjunctival tissue scrapings; specific IgE antibody level (titer) in conjunctival tissue scrapings or in serum; and basophil histamine release in blood.

In some embodiments, the one or more symptoms may be symptoms associated with atopic dermatitis. For example, the method may comprise reducing one or more of the following symptoms: itch intensity of the skin; eczema score, and number of (peripheral) blood eosinophils.

In some embodiments, the one or more symptoms may be symptoms associated with allergic angioedema. For example, the method may comprise reducing one or more of the following symptoms: swelling, shortness of breath, dizziness, fainting, abdominal pain from swelling of the intestinal tract, urticaria, changes in blood pressure.

A therapeutic or beneficial effect also includes reducing or eliminating the need, dosage frequency or amount of a second therapeutic method or therapeutically active drug (e.g., anti-inflammatory, decongestants or anti-allergic agent) used for treating a subject having an immune response or one or more symptoms caused by or associated with an allergen. For example, administration of a vaccine described herein may reduce the amount of an adjunct therapy administered to a subject, such as reducing the subject's need for concomitant treatment with fast or long-acting (32-agonists, leukotriene modifiers, theophylline corticosteroids or HI antihistamines (e.g., inhaled, or oral) to reduce, relieve, or suppress one or more symptoms of the immune response.

As used herein, the term “immune response” includes T cell (cellular) mediated and/or B cell (humoral) mediated immune responses, or both cellular and humoral responses. In particular, the term “immune response” may include an IgE-mediated immune response (i.e., an allergic immune response). Exemplary immune responses include T cell responses, such as Th2 responses resulting in cytokine production and/or cellular cytotoxicity. In addition, the term “immune response” includes responses that are indirectly affected by T cell activation, e.g., antibody production (humoral responses) and activation of cytokine responsive cells, e.g., eosinophils, macrophages.

Immune cells involved in the immune response include lymphocytes, such as T cells (CD4+, CD8+, Th1 and Th2 cells, memory T cells) and B cells; antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer (NK) cells; and myeloid cells, such as macrophages, eosinophils, mast cells, basophils, and granulocytes. A particular immune response is production of immunoglobulin (Ig) isotypes antibodies or decreasing IgE antibodies.

Therefore, in some embodiments, the method of treatment comprises inducing or increasing an IgG antibody (e.g., specific IgG) response in a subject to an allergen, such as but not limited to food-pollen allergens, such as but not limited to Pru P 3 and similar allergens. In still some embodiments, the method comprises decreasing an IgE antibody (e.g. specific IgE) response in a subject to an allergen of a food and/or pollen. In still some embodiments, the method comprises decreasing a T cell response in a subject to an allergen, preferably, from food and/or pollen, for example by decreasing the production of Th-2 associated cytokines, like IL-5, IL-4, IL-13 in response to said allergen.

The term “modulating an immune response” or “modulate an immune response” may include to stimulate, induce, promote, increase or enhance an immune response, e.g., a T cell regulatory response, or may include inhibiting, decreasing, suppressing or reducing a T cell response, which may include, but is not limited to a Th2 cell response.

As mentioned, vaccine described herein may provide a beneficial effect on an immune response against food and/or pollen allergies.

Typically, the treatment comprises repeated administration of the composition with weekly, bi weekly, monthly or quarterly intervals. Thus, in a particular embodiment, the treatment comprises immunotherapy with single doses repeatedly administered until a persistent effect is achieved. Immunotherapy is thought to produce immunological tolerance in the subject undergoing therapy.

Thus, in still other embodiments, the compositions, such as peptide combinations, may be used to induce immunological tolerance in a subject in need thereof.

As used herein, the term “immunological tolerance” refers to a) a decreased or reduced level of a specific immunological response (thought to be mediated at least in part by antigen-specific effector T lymphocytes, B lymphocytes, antibodies, or a combination thereof); b) a delay in the onset or progression of a specific immunological response; or c) a reduced risk of the onset or progression of a specific immunological response (e.g. to a food allergen or pollen allergen). An increase, improvement, enhancement or induction of “tolerance” may refer to a decrease, reduction, inhibition, suppression, or limiting or controlling or clearing of specific immunological reactivity to an allergen as compared to reactivity to the allergen in a previous exposure to the same allergen. Thus, in certain embodiments, the method comprises inducing immunological tolerance in a subject to an allergen (e.g., a food or pollen allergen) to suppress an allergic immune response to the allergen.

Immunological tolerance in a subject to an allergen can also be reflected by reducing the occurrence, frequency, severity, progression, or duration of an allergic response of the subject to the allergen.

Induction of immune tolerance (also referred to as desensitization), and the relative amount of immune tolerance, can be measured by methods disclosed herein or known to the skilled artisan. For example, induction of immune tolerance can be measured by the modulated lymphokine and/or cytokine level in a subject or animal before versus after administering a peptide combination described herein for the first time. A modulated cytokine level can be an increase of a cytokine level, for instance an increase of a lymphokine and/or cytokine level of at least 1.5, 2, 3, 4, 5, 6, 7, 8, 10, 20, 50 times or more relative to before administering the vaccine for the first time. Accordingly, the term “inducing immunological tolerance” may include eliciting, stimulating, promoting, increasing, or enhancing immunological tolerance. Immunological tolerance may involve modulation of T cell activity, including but not limited to CD4+ T cells, CD8+ T cells, Th1 cells, Th2 cells and regulatory T cells (Tregs), and memory T cells, including inflammatory lymphokines/cytokines produced by T cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### Sequences

Table 4 describes the sequences of the present invention.

TABLE 4

| SEQ ID NO | Type | Note |
|---|---|---|
| 1 | Amino acid | Consensus allergen CA1 |
| 2 | Amino acid | Consensus allergen CA2 |
| 3 | Amino acid | Consensus allergen CA3 |
| 4 | Amino acid | Consensus allergen CA4 |
| 5 | Nucleic acid/ | DNA sequence encoding CA1 optimized for expression in homo |
| | DNA | sapiens. |
| 6 | Nucleic acid/ | DNA sequence encoding CA1 optimized for expression in homo |
| | DNA | sapiens using consensus codons |
| 7 | Nucleic acid/ | DNA sequence encoding CA2 optimized for expression in homo |
| | DNA | sapiens. |
| 8 | Nucleic acid/ | DNA sequence encoding CA2 optimized for expression in homo |
| | DNA | sapiens using consensus codons |
| 9 | Nucleic acid/ | DNA sequence encoding CA3 optimized for expression in homo |
| | DNA | sapiens. |
| 10 | Nucleic acid/ | DNA sequence encoding CA3 optimized for expression in homo |
| | DNA | sapiens using consensus codons |
| 11 | Nucleic acid/ | DNA sequence encoding CA4 optimized for expression in homo |
| | DNA | sapiens. |
| 12 | Nucleic acid/ | DNA sequence encoding CA4 optimized for expression in homo |
| | DNA | sapiens using consensus codons |
| 13 | Nucleic acid/ | RNA sequence encoding CA1 optimized for expression in homo |
| | RNA | sapiens |
| 14 | Nucleic acid/ | RNA sequence encoding CA2 optimized for expression in homo |
| | RNA | sapiens |
| 15 | Nucleic acid/ | RNA sequence encoding CA3 optimized for expression in homo |
| | RNA | sapiens |
| 16 | Nucleic acid/ | RNA sequence encoding CA4 optimized for expression in homo |
| | RNA | sapiens |
| 17 | Amino acid | Pru P 3 from Prunus persica, a marker allergen for lipid transfer |
| | | protein sensitization |
| 18 | Amino acid | Jur r 3 from Juglans regia, a marker allergen for lipid transfer |
| | | protein sensitization |
| 19 | Amino acid | Mal d 3 from Malus domestica, a non-specific lipid transfer protein |
| | | type 1 (nsLTP1) and is an allergen of the apple. |
| 20 | Amino acid | Consensus allergen CA8 |
| 21 | Amino acid | Consensus allergen CA 6 |
| 22 | Amino acid | Consensus allergen 5 |
| 23 | Amino acid | Consensus allergen 7 |

### EXAMPLES

### Example 1

### Identification of Pru p 3 Protein Allergens for Generation of Sequence Set 1

The amino acid sequences of the known wild-type protein allergen Pru p 3 (SEQ ID NO: 17) was aligned to known sequences in the ALLERGOME database (http://www.allergome.org/script/tools.php?tool=blaster) using the BLAST algorithm, with the following parameters:
Algorithm: NCBI blastp
Matrix: BLOSUM62
Gap costs: Open: 11 Ext: 1
Expect threshold: 100
Word size: 3

The identified sequences were extracted from the blast search and aligned using Jalview 2.11.2.0 (https://www.jalview.org).

Following alignment of the sequences, the hits were manually curated and sequences, wherein the alignment was less than 80% of the total length, were eliminated from the set used to generate the consensus allergen. For CA1, the sequences available at the following uniport identifier were used to generate the consensus allergen: A0A059SSZ0, A0A059ST23, A0A059STC4, B6CEX8, B6CG41, C4MGG9, C4MGH0, C4MGH1, C5H617, E6Y2L9, O04403, P0C088, P19656, P55958, P81402, P85204, P93224, Q4PLT6, Q4PLT9, Q4PLU0, Q5IZZ5, Q5IZZ6, Q5J009, Q5J011, Q5J026, Q8VX12, Q9ATH2 and W0U0V5.

### Identification of Cross-Recognized Allergens for Generation of Sequence Set 2

The input sequences for the generation of CA2 with the amino acid sequence of SEQ ID NO: 2, was prepared from the known allergenic and cross-recognized allergens based on their WHO nomenclature as provided Skypala et. al. 2021 (Skypala et. al. 2021, Non-specific lipid-transfer proteins: Allergen structure and function, cross-reactivity, sensitization, and epidemiology, Clinical and Translational Allergy Volume 11, Issue 3).

For CA2, the sequences available at the following uniport identifier were used to generate the consensus allergen: O04004, E6Y8S8, B6CEX8, W0U0V5, Q9ATH2, Q8VX12, P82007, Q8RYA8, C5H617, A0AT29, Q5J026, P85894, D3W146, P81651, Q9M5X8, P82534, COL0I5, P81402, A0A059STC4, Q9M5X6, Q0Z8V0, E6Y2L9, P93224, D2T2K2, Q850K5 and P19656

### Identification of Cross-Recognized Allergens for Generating of Sequence Set 3

The input sequences for the generation of CA3 with the amino acid sequence of SEQ ID NO: 3, were prepared from the known allergenic and cross-recognized allergens based including distant species as provided by O'Malley et al., 2021 (O'Malley et al., 2021, Structural Characterization of Act c 10.0101 and Pun g 1.0101—Allergens from the Non-Specific Lipid Transfer Protein Family, Molecules 2021, 26(2), 256).

For CA3, the sequences available at the following uniport identifier was used to generate the consensus allergen: A0A059SSZ0, A0A059ST23, A0A059STC4, A0A1J7GK90, A0A4P1RWD8, A0AT29, A0AT32, A0AT33, A1E2H5, A9YUH6, B6CEX8, B6CG41, B6SGP7, B6TTP1, C5H617, D3W146, D3W147, E7CLQ2, E7CLQ4, E7CLQ5, E7CLQ6, E7CLQ7, E7CLQ8, E7CLR2, F1AHA2, F6GXX3, G8DM17, G8DM18, G8DM19, G8DM20, I6QLE1, M4QHL5, M4QL90, M4QUI6, O23758, P19656, P81402, P81651, P82534, P85894, Q0Z8V0, Q2QCI7, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q43017, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q6EV47, Q6TKQ7, Q850K5, Q850K6, Q8H2B2, Q8L5S8, Q8RYA8, Q8VX12, Q9LED1, Q9M5X6, Q9M5X7, Q9M5X8 and W0U0V5.

### Identification of Jur r 3 Cross-Recognized Allergens for Generation of Sequence Set 4

The amino acid sequences of the known wild-type protein allergen Jur r 3 (SEQ ID NO: 18) was aligned to known sequences in the ALLERGOME database (http://www.allergome.org/script/tools.php?tool=blaster) using the BLAST algorithm, with the following parameters:
Algorithm: NCBI blastp
Matrix: BLOSUM62
Gap costs: Open: 11 Ext: 1
Expect threshold: 100
Word size: 3

The identified sequences were extracted from the blast search and aligned using JALview 2.11.2.0 (https://www.jalview.org).

Following alignment of the sequences, the hits were manually curated and sequences with less than 60% amino acid sequence identity to Jur r 3 and sequences with less than 90 amino acids were eliminated from the set used to generate the consensus allergen. For CA4, the sequences available at the following uniport identifier was used to generate the consensus allergen: A0A059SSZ0, A0A059ST23, A0A059STC4, A0A158V755, A0A161AT60, A0A1J7GK90, A0A1W5LDB3, A0A1W5LDC0, A0A1W5LDC1, A0A1W5LDC2, A0A1W5LE45, A0A1W5LG02, A0A445AL51, A0A4P1RWD8, A0A510A9S3, A0AT28, A0AT29, A0AT30, A0AT31, A0AT32, A0AT33, A1E2H4, A1E2H5, A2ZAS9, A2ZAT1, A2ZDR8, A2ZHF1, A3C7Z3, A8YPK3, A9YUH6, B6CEX8, B6CG41, B6CQU4, B6CQU6, B6CQU7, B6SGP7, B6SY96, B6T089, B6TTP1, B8QW29, B8QW30, B8QW32, B8QW33, B8QW34, B8QW37, B8QW40, B8QW53, B8QW56, B8QW58, B8QW69, B8QW75, B8QW95, B8QWA1, COL0I5, C4MGG9, C4MGH0, C4MGH1, C4MGH2, C5H617, D2T0A5, D2T0A6, D2T2K0, D2T2K1, D2T2K2, D3W146, D3W147, D4QD83, E6Y2L9, E6Y8S8, E7CLQ2, E7CLQ4, E7CLQ5, E7CLQ6, E7CLQ7, E7CLQ8, E7CLR2, F1AHA2, F2CY84, F2ED95, F6GXX3, F6MEX1, G8DM17, G8DM18, G8DM19, G8DM20, I6QLE1, M0V3U0, M1CHX3, M4QHL5, M4QL90, M4QUI6, O04004, O04403, O04404, O22482, O22485, O23758, O65091, P19656, P24296, P27056, P27631, P43217, P55958, P81402, P81651, P82007, P82534, P85206, P85894, P86137, P93224, Q0IQK9, Q0Z8V0, Q14K71, Q1JTN5, Q2PCB7, Q2PCB8, Q2PCD1, Q2PCD2, Q2QCI7, Q2QYL2, Q2QYL3, Q2RBD2, Q2V6D8, Q2XX13, Q2XX14, Q2XX15, Q2XX16, Q2XX17, Q2XX18, Q2XX19, Q2XX21, Q2XX22, Q2XX23, Q2XX24, Q2XX25, Q2XX37, Q2XX39, Q2XX47, Q2XX49, Q39382, Q40905, Q42589, Q43017, Q4A1N0, Q4A1N1, Q4PLT5, Q4PLT6, Q4PLT7, Q4PLT8, Q4PLT9, Q4PLU0, Q4VUZ0, Q5GLH0, Q5IZZ5, Q5IZZ6, Q5J000, Q5J009, Q5J011, Q5J026, Q5NE26, Q5NE27, Q5NE31, Q6EV47, Q6TKQ7, Q7XJ39, Q850K5, Q850K6, Q8GZB0, Q8H2B2, Q8L5S8, Q8RYA8, Q8VX12, Q9ATH2, Q9LED1, Q9M5X6, Q9M5X7, Q9MSX8, Q9S713 and W0U0V5.

### Identification of Bet v 1 Protein Allergens for Generation of Sequence Set 5

The amino acid sequences of the known wild-type protein allergen Bet v 1 (SEQ ID N022) was aligned to known sequences in the ALLERGOME database (http://www.allergome.org/script/tools.php?tool=blaster) using the BLAST algorithm, with the following parameters:
Algorithm: NCBI blastp
Matrix: BLOSUM62
Gap costs: Open: 11 Ext: 1
Expect threshold: 100
Word size: 3

The identified sequences were extracted from the blast search and aligned using Jalview 2.11.2.0 (https://www.jalview.org).

Following alignment of the sequences, the hits were manually curated and sequences, wherein the alignment was less than 80% of the total length, were eliminated from the set used to generate the consensus allergen. For CA5 the sequences available at the following uniport identifier were used to generate the consensus allergen: Q546U3, P15494, Q96366, O24642, Q42499, Q546V0, P15494, Q9SCH8, 023752, Q96371, Q96370, Q9SCI0, Q9SCH9, Q96365, Q9SYW1, Q96367, Q9SCI3, Q9SYW0, Q9SCI2, O23753, Q9AYS2, O23754, O23751, Q96368, P43183, Q9AYS3, Q39431, P43177, Q39426, P43179, P43180, Q9SYW2, Q9SCH5, Q9ZS39, O23748, ref:19841, P43185, Q546V1, P43178, Q9AYS4, ref:10368, O23746, Q9SCH6, O23750, C0IVP0, Q0QKX7, Q0QLT3, C0IVP2, C0IVP5, C0IVQ6, C0IVP3, Q0QLW3, C0IVP6, C0IVQ7, Q9ZS38, C0IVR6, C0IVP1, C0IVP4, C0IVR1, C0IVR7, Q0QLS9, C0IVQ4, Q0QLT1, C0IVR2, C0IVR5, ref:10368, Q0QLS8, Q39429, C0IVP8, Q0QLW1, C0IVS1, Q39428, P43186, C0IVS3, Q0QLV9, Q0QLV8, Q0QLW0, Q39427, C0IVR8, C0IVR9, C0IVR4, C0IVP9, C0IVQ1, C0IVQ2, Q0QKX5, C0IVR0, Q0QLT0, O23749, C0IVQ3, P45431, C0IVS2, C0IVS4, P43184, O23747, C0IVS0, P43176, C0IVQ8, C0IVQ9, C0IVR3, Q9LEP0, Q39430, Q39420, Q39453, C0IVT2, Q0QKX4, C0IVT9, C0IVT4, Q39425, C0IVS9, C0IVS8, Q0QLV3, Q0QLV6, C0IVT8, Q0QLV2, C0IVU1, C0IVU0, C0IVS6, P38948, C0IVU4, Q0QLS7, C0IVS5, C0IVU2, C0IVT7, C0IVU5, Q0QLV0, C0IVT0, Q0QLV5, C0IVT6, Q0QLS6, C0IVX8, C0IVX7, C0IVY4, C0IVW8, Q0QLS2, C0IVU3, C0IVW6, C0IVW2, C0IVY0, C0IVX2, C0IW4, C0IW6, Q0QLS5, C0IVX9, C0IW8, Q0QLU8, C0IVT5, C0IVW0, Q0QKX2, C0IVX4, Q0QLU7, C0IW5, C0IVW7, Q96382, C0IVW9, C0IVX3, C0IVY2, C0IVY3, C0IW2, C0IVX6, C0IW1, C0IVU9, C0IVW3, C0IW0, Q0QKW9, Q0QLU2, C0IW3, C0IVW5, C0IVU7, C0IVU6, Q0QKX1, P38950, Q96381, C0IW11, Q96378, Q96377, B6RQR6, B6RQR9, B6RQR8, B6RQR7, E2GL17, Q08407, B6RQS0, Q96379, Q08407, Q96503, Q08407, P38949, P38949, Q96501, Q9SCI1, Q39415, Q08407, Q9SCH7, Q96380, H9NJ55, H9NJ58, Q39454, H9NJ59, H9NJ57, Q9ZRU8, H9NJ56, Q9SWR4, C0IW05, C0IW03, C0IW04, C0IVZ8, C0IW07, C0IW00, Q0QLT5, C0IVZ5, C0IW09, Q9FPK2, Q93YH9, Q0QLT4, Q9FPK4, Q9FPK3, C0IVZ1, Q0QLT9, C0IVZ3, C0IVZ0, C0IVY9, C0IW10, C0IVY6, C0IVY7, Q0QKW8, A8W7B6, B7TWE8, B7TWE7, C0IVZ4, C0IVZ2, B7TWE6, Q43550, A0A1J0RET5, Q43551, Q43552, A0AUB6, Q4VPL0, F6LXS7, A0AUB7, A0AUB8, B6CQS5, B6CQS6, O24248, Q43549, D7SY82, A5B0T9, O50001, A5C113, B6CQR8 and Q2I6V8.

### Identification of Mal d 1 Protein Allergens for Generation of Sequence Set 6

The amino acid sequences of the known wild-type protein allergen Mal d 1 (SEQ ID NO: 21) was aligned to known sequences in the ALLERGOME database (http://www.allergome.org/script/tools.php?tool=blaster) using the BLAST algorithm, with the following parameters:
Algorithm: NCBI blastp
Matrix: BLOSUM62
Gap costs: Open: 11 Ext: 1
Expect threshold: 100
Word size: 3

The identified sequences were extracted from the blast search and aligned using Jalview 2.11.2.0 (https://www.jalview.org).

Following alignment of the sequences, the hits were manually curated and sequences, wherein the alignment was less than 80% of the total length, were eliminated from the set used to generate the consensus allergen. For CA6 the sequences available at the following uniport identifier were used to generate the consensus allergen:

P43211, Q9SYW3, Q941P6, Q9SYV7, Q9SYV2, Q9SYV6, Q9SYV5, F5CEW9, Q9SYV8, P43211, 065200, Q9S7M5, Q40280, Q9SYV4, Q40280, A0AUB1, Q941P5, Q9SYV3, F6M053, Q9SYV9, Q8L6K9, O24248, B5KVN9, B5KVP1, B6CQR8, Q2I6V8, B6CQR7, O22521, B7VFN6, Q84LA7, Q4VPI9, Q941P8, Q5VJR0, Q4VPI6, Q4VPJ0, Q256S4, Q5VJR1, Q4VPI3, Q4VPJ5, Q4VPI0, Q5VJQ9, Q43549, Q256S7, Q3T923, Q941P7, Q5VJQ8, Q256S2, Q5VJR5, Q4VPI7, Q256S6, B6CQS3, Q5VJR2, B0B0L6, A0AUF9, Q4VPJ8, B0B0L5, A0AUG8, M5XFW3, B6CQS4, Q4VPK5, Q4VPJ9, F6LWG3, F6LWG8, A0AUE3, A0AU76, A0AU75, F6LWG7, B6CQS1, B0B0L9, A0A1J0RET5, Q5VJR3, Q4VPH9, A0AUG9, B0B0M5, Q4VPK0, A0AUF7, E4Z8P8, B6CQS2, F6LWF9, Q4VPJ7, G8E012, B6CQS5, Q5VJR4, B6CQR9, O50001, F6LWG6, ref:23394, D0E0C7, A0AUB7, B6CQS6, A0AUH0, Q4VPL0, Q5VJQ7, Q4VPK7, F6LXS7, A0AUC8, ref:23394, ref:23394, G8H6R0, A0AUB8, A0AUB6, H9NJ57, D0E0C6, ref:23394, Q43552, Q9ZRU8, Q43551, ref:23394, Q43550, B6CQT0, H9NJ59, Q4VPK6, ref:23394, F6KDF1, H9NJ55, ref:23394, H9NJ58, Q93YH9, H9NJ56, ref:23394, ref:23394, M5XTC6, B6CQT1, Q6QHU2, ref:23394, B6CQS7, ref:23394, ref:13687, ref:23394, ref:23394, B6CQS9, ref:23394, B7TWE7, Q6QHU3, B7TWE6, Q6QHU1, Q4VPJ1, A0AU70, B7TWE8, A0AU71, A5C113, A5B0T9, D7SY82, ref:23394, A5C112, D7SY83, Q4VPJ3, Q39454, Q39415, Q9SWR4, A8W7B6, Q9FPK4, Q9FPK2, F6H6U9, Q9FPK3, A5CAV3, B7TWE3, F6H6U5, B7TWE5, A5AQ75, B6RQS2, B7TWE4, Q9FS42, Q39427, D7SY74, A0A2H5CUG2, P43186, Q9LEP0, Q39429, Q39428, O23747, O23749, Q39420, Q0Z8U9, P43176, B6RQS3, P43184, H9NJ54, P45431, Q0QLT4, Q39453, Q9ZS38, C0IW05, C0IW03, C0IW04, C0IVZ8, C0IW07, C0IW00, Q0QLT5, B9RTC1, ref:19841, C0IW09, Q9SYW2, Q9SCH6, C0IW10, Q9SCH5, B6RQS1, Q9ZS39, Q39430, B9RTC5, Q9SCH9, Q96370, O23748, O23751, O23752, Q9SYW0, O23754, Q9AYS2, Q9SCI0, Q39426, Q96365, O24642, P38948, P43180, Q08407, P43177, Q42499, Q96366, O23746, Q546U3, P15494, P43183, Q96371, Q9SCH8, O23753, P43178, Q9AYS4, Q9SCI2, Q9SCI3, C0IVS9, C0IVS8, Q0QLV3, Q96367, P43179, Q0QLU7, Q39425, P43185, C0IVX8, C0IVX7, Q96368, C0IVY2, Q96382, C0IVV8, Q0QLU8, C0IVT2, Q0QKX4, Q9AYS3, C0IVW2, C0IVY0 and C0IVX2.

### Identification of Cross-Recognized Allergens for Generating of Sequence Set 7

The amino acid sequences of the known wild-type protein allergen Act c 8(SEQ ID NO: 23) was aligned to known sequences in the ALLERGOME database (http://www.allergome.org/script/tools.php?tool=blaster) using the BLAST algorithm, with the following parameters:
Algorithm: NCBI blastp
Matrix: BLOSUM62
Gap costs: Open: 11 Ext: 1
Expect threshold: 100
Word size: 3

The identified sequences were extracted from the blast search and aligned using Jalview 2.11.2.0 (https://www.jalview.org).

Following alignment of the sequences, the hits were manually curated and sequences, wherein the alignment was less than 80% of the total length, were eliminated from the set used to generate the consensus allergen. For CA7 the sequences available at the following uniport identifier were used to generate the consensus allergen: B6CQR8, Q2I6V8, B6CQR7, B5KVN9, B5KVP1, O22521, Q9S7M5, Q9SYV4, A0AUB1, F6M053, Q9SYV9, Q941P5, Q9SYV3, Q8L6K9, Q9SYV2, Q9SYV5, Q9SYW3, Q9SYV8, Q9SYV6, Q9SYV7, Q941P6, Q4VPL0, F5CEW9, F6LXS7, Q4VPK7, A0AUB7, B6CQS1, A0AUB6, A0AUB8, A0AUC8, Q43552, M5XFW3, Q4VPK6, B6CQR9, Q43550, Q43551, B6CQS2, Q84LA7, Q5VJQ7, Q4VPI9, B6CQS5, Q4VPI0, Q4VPI6, Q5VJR0, B6CQS6, Q4VPJ0, Q941P8, Q43549, Q5VJR1, Q4VPI3, Q4VPI7, G8H6R0, Q4VPH9, E4Z8P8, Q4VPJ5, H9NJ57, Q5VJQ8, Q941P7, B6CQT0, Q5VJQ9, B6CQS4, B6CQS3, M5XTC6, H9NJ55, B6CQT1, A0A1J0RET5, Q6QHU2, A0AU76, B6CQS7, B7VFN6, H9NJ56, H9NJ58, B6CQS9, Q6QHU3, Q4VPK5, Q9ZRU8, A0AUE3, A0AU75, Q6QHU1, B7TWE7, B0B0L9, Q5VJR5, B0B0M5, B7TWE6, B7TWE8, B0B0L6, A0AUG8, Q4VPJ8, B0B0L5, Q4VPJ9, Q5VJR2, A0AUF9, Q5VJR4, F6LWG3, F6LWG8, D7SY83, A5C113, F6LWG7, A0AUG9, Q93YH9, Q5VJR3, Q4VPK0, F6LWF9, A0AUF7, Q4VPJ7, A5B0T9, G8E012, D7SY82, H9NJ59, F6LWG6, F6KDF1, A0AUH0, A5C112, A0AU70, A0AU71, Q4VPJ1, F6H6U9, B6RQS2, Q39454, A5CAV3, Q4VPJ3, B7TWE4, A5AQ75, Q9FS42, B7TWE3, D7SY74, B7TWE5, A0A2H5CUG2, Q39415, F6H6U5, Q9SWR4, B6RQS3, Q9FPK3, Q39427, Q9FPK4, Q9FPK2, B9RTC1, H9NJ54, O23747, A8W7B6, O23749, Q39429, Q39428, B6RQS1, Q9LEP0, Q39453, Q39420, Q0QLT4, Q9ZS38, Q96370, C0IW09, C0IW05, C0IW03, C0IW04, C0IVZ8, C0IW07, C0IW00, Q0QLT5, Q9ZS39, O23752, Q9SCH6, O23754, Q0Z8U9, C0IW10, O24642, Q9SYW2, Q9SCI2, Q9SCH5, Q42499, Q546U3, O23753, O23748, Q96371, Q96366, Q39426, Q9SCH8, Q39430, Q96382, O23751, Q9SCH9, Q546V0, Q9SYW0, D1YSM4, Q96378, Q9SCI0, Q96365, C0IVT2, Q0QKX4, C0IVS9, C0IVS8, Q0QLV3, Q9AYS2, Q0QLS7, Q39431, Q96367, Q9AYS4, Q9SCI3, C0IVT9, C0IVT4, Q9SYW1, B9RTC5, Q96381, Q0QLV6, Q9AYS3, D1YSM5, E9M219, B6RQR9, B6RQR6, Q96377, B6RQR7, E2GL17, B6RQS0, B6RQR8, Q96503, Q96379, Q39425, K4CWC4, Q96501, E9M220, C0IVZ2, Q96380, C0IVZ5, Q0QLU7, C0IVZ0, C0IVY9, C0IVZ3, C0IVY2, C0IVY6, C0IVY7, Q0QKW8, C0IVZ1, Q0QLT9, Q96368, Q546V1, O23746, O23750, C0IVP0, Q0QKX7, Q0QLT3, C0IVP2, C0IVP5, C0IVQ6, C0IVP3, Q0QLW3, C0IVP6, C0IVQ7, C0IVR6, C0IVP1, C0IVP4, C0IVR1, C0IVR7, Q0QLS9, C0IVQ4, Q0QLT1, C0IVR2, C0IVR5, Q0QLS8, C0IVP8, Q0QLW1, C0IVS1, C0IVS3, Q0QLV9, Q0QLV8, Q0QLW0, C0IVR8, C0IVR9, C0IVR4, C0IVP9, C0IVQ1, C0IVQ2, Q0QKX5, C0IVR0, Q0QLT0, C0IVQ3, C0IVS2, C0IVS4, C0IVS0, C0IVQ8, C0IVQ9, C0IVR3, C0IVT8, Q0QLV2, C0IVU1, C0IVU0, C0IVS6, C0IVU4, C0IVS5, C0IVU2, C0IVT7, C0IVU5, Q0QLV0, C0IVT0, Q0QLV5, C0IVT6, Q0QLS6, C0IVX8, C0IVX7, C0IVY4, C0IVW8, Q0QLS2, C0IVU3, C0IVW6, C0IVW2, C0IVY0, C0IVX2, C0IVV4, C0IW6, Q0QLS5, C0IVX9, C0IW8, Q0QLU8, C0IVT5, C0IVW0, Q0QKX2, C0IVX4, C0IVV5, C0IVW7, C0IVW9, C0IVX3, C0IVY3, C0IVV2, C0IVX6, C0IW1, C0IVU9, C0IVW3, C0IVV0, Q0QKW9, Q0QLU2, C0IVV3, C0IVW5, C0IVU7, C0IVU6, Q0QKX1, C0IW11, Q9SCI1, Q9SCH7 and C0IVZ4.

### Identification of Cross-Recognized Allergens for Generation of Sequence Set 8

The curated list of sequences extracted for building CA5, CA6, and CA7 were merged and duplicated sequences were eliminated from the pool.

The identified sequences were extracted from the blast search and aligned using Jalview 2.11.2.0 (https://www.jalview.org).

Following alignment of the sequences, the hits were manually curated and sequences, wherein the alignment was less than 80% of the total length, were eliminated from the set used to generate the consensus allergen. For CA8 the sequences available at the following uniport identifier were used to generate the consensus allergen: B6CQR8, Q2I6V8, B6CQR7, B5KVN9, B5KVP1, O22521, Q9S7M5, Q9SYV4, A0AUB1, F6M053, Q9SYV9, Q941P5, Q9SYV3, Q8L6K9, Q9SYV2, Q9SYV5, Q9SYW3, Q9SYV8, Q9SYV6, Q9SYV7, Q941P6, Q4VPL0, F5CEW9, F6LXS7, Q4VPK7, A0AUB7, B6CQS1, A0AUB6, A0AUB8, A0AUC8, Q43552, M5XFW3, Q4VPK6, B6CQR9, Q43550, Q43551, B6CQS2, Q84LA7, Q5VJQ7, Q4VPI9, B6CQS5, Q4VPI0, Q4VPI6, Q5VJR0, B6CQS6, Q4VPJ0, Q941P8, Q43549, Q5VJR1, Q4VPI3, Q4VPI7, G8H6R0, Q4VPH9, E4Z8P8, Q4VPJ5, H9NJ57, Q5VJQ8, Q941P7, B6CQT0, Q5VJQ9, B6CQS4, B6CQS3, M5XTC6, H9NJ55, B6CQT1, A0A1J0RET5, Q6QHU2, A0AU76, B6CQS7, B7VFN6, H9NJ56, H9NJ58, B6CQS9, Q6QHU3, Q4VPK5, Q9ZRU8, A0AUE3, A0AU75, Q6QHU1, B7TWE7, B0B0L9, Q5VJR5, B0B0M5, B7TWE6, B7TWE8, B0B0L6, A0AUG8, Q4VPJ8, B0B0L5, Q4VPJ9, Q5VJR2, A0AUF9, Q5VJR4, F6LWG3, F6LWG8, D7SY83, A5C113, F6LWG7, A0AUG9, Q93YH9, Q5VJR3, Q4VPK0, F6LWF9, A0AUF7, Q4VPJ7, A5B0T9, G8E012, D7SY82, H9NJ59, F6LWG6, F6KDF1, A0AUH0, A5C112, A0AU70, A0AU71, Q4VPJ1, F6H6U9, B6RQS2, Q39454, A5CAV3, Q4VPJ3, B7TWE4, A5AQ75, Q9FS42, B7TWE3, D7SY74, B7TWE5, A0A2H5CUG2, Q39415, F6H6U5, Q9SWR4, B6RQS3, Q9FPK3, Q39427, Q9FPK4, Q9FPK2, B9RTC1, H9NJ54, O23747, A8W7B6, O23749, Q39429, Q39428, B6RQS1, Q9LEP0, Q39453, Q39420, Q0QLT4, Q9ZS38, Q96370, C0IW09, C0IW05, C0IW03, C0IW04, C0IVZ8, C0IW07, C0IW00, Q0QLT5, Q9ZS39, O23752, Q9SCH6, O23754, Q0Z8U9, C0IW10, O24642, Q9SYW2, Q9SCI2, Q9SCH5, Q42499, Q546U3, O23753, O23748, Q96371, Q96366, Q39426, Q9SCH8, Q39430, Q96382, O23751, Q9SCH9, Q546V0, Q9SYW0, D1YSM4, Q96378, Q9SCI0, Q96365, C0IVT2, Q0QKX4, C0IVS9, C0IVS8, Q0QLV3, Q9AYS2, Q0QLS7, Q39431, Q96367, Q9AYS4, Q9SCI3, C0IVT9, C0IVT4, Q9SYW1, B9RTC5, Q96381, Q0QLV6, Q9AYS3, D1YSM5, E9M219, B6RQR9, B6RQR6, Q96377, B6RQR7, E2GL17, B6RQS0, B6RQR8, Q96503, Q96379, Q39425, K4CWC4, Q96501, E9M220, C0IVZ2, Q96380, C0IVZ5, Q0QLU7, C0IVZ0, C0IVY9, C0IVZ3, C0IVY2, C0IVY6, C0IVY7, Q0QKW8, C0IVZ1, Q0QLT9, Q96368, Q546V1, O23746, O23750, C0IVP0, Q0QKX7, Q0QLT3, C0IVP2, C0IVP5, C0IVQ6, C0IVP3, Q0QLW3, C0IVP6, C0IVQ7, C0IVR6, C0IVP1, C0IVP4, C0IVR1, C0IVR7, Q0QLS9, C0IVQ4, Q0QLT1, C0IVR2, C0IVR5, Q0QLS8, C0IVP8, Q0QLW1, C0IVS1, C0IVS3, Q0QLV9, Q0QLV8, Q0QLW0, C0IVR8, C0IVR9, C0IVR4, C0IVP9, C0IVQ1, C0IVQ2, Q0QKX5, C0IVR0, Q0QLT0, C0IVQ3, C0IVS2, C0IVS4, C0IVS0, C0IVQ8, C0IVQ9, C0IVR3, C0IVT8, Q0QLV2, C0IVU1, C0IVU0, C0IVS6, C0IVU4, C0IVS5, C0IVU2, C0IVT7, C0IVU5, Q0QLV0, C0IVT0, Q0QLV5, C0IVT6, Q0QLS6, C0IVX8, C0IVX7, C0IVY4, C0IVW8, Q0QLS2, C0IVU3, C0IVW6, C0IVW2, C0IVY0, C0IVX2, C0IVV4, C0IVV6, Q0QLS5, C0IVX9, C0IVV8, Q0QLU8, C0IVT5, C0IVW0, Q0QKX2, C0IVX4, C0IVV5, C0IVW7, C0IVW9, C0IVX3, C0IVY3, C0IVV2, C0IVX6, C0IW1, C0IVU9, C0IVW3, C0IVV0, Q0QKW9, Q0QLU2, C0IVV3, C0IVW5, C0IVU7, C0IVU6, Q0QKX1, C0IW11, Q9SCI1, Q9SCH7 and C0IVZ4.

### Example 2

Generation of CA1-4 from Sequence Set 1-4

To obtain CA1 of SEQ ID NO: 1, sequence set 1 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

To obtain CA2 of SEQ ID NO: 2, sequence set 2 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

To obtain CA3 of SEQ ID NO: 3, sequence set 2 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

To obtain CA4 of SEQ ID NO: 4, sequence set 2 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

Generation of CA5-5 from Sequence Set 5-8

To obtain CA5 (PR10-3) of SEQ ID NO: 22, sequence set 5 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

To obtain CA6 (PR10-2) of SEQ ID NO: 21, sequence set 6 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

To obtain CA7 (PR10-4) of SEQ ID NO: 23, sequence set 7 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

To obtain CA8 (PR10-1) of SEQ ID NO: 20, sequence set 8 was aligned and the consensus sequence was determined based on the incidence number of each amino acid in each specific position in the aligned sequence. When two amino acids were equally represented in a position, or in case of a gap in the aligned sequence, the amino acid with the highest molecular volume, as defined in table 3, was selected as the conserved amino acid. In the cases where the two amino acids remain equally represented following selection of the amino acid with the highest molecular volume, the conserved amino acid was selected based on the physicochemical properties of amino acid according based on the selection criteria defined in Table 2.

### Example 3

In this experiment, we demonstrate that the designed consensus allergens (CAs) conserve epitopes that can be recognized by IgEs present in the serum of Peach-Cypress syndrome patients.

### Materials and Methods

CAs cDNA sequences optimized for yeast expression (GeneScript) were purchased (Eurofins genomics) and subcloned into the pPICZαA vector and electroporated into Pichia pastoris KM71H electrocompetent cells. After 72 h of methanol induction, recombinant CAs were isolated from extracellular culture and purified throughout a cation-exchange chromatography (UNO-SphereS—BioRad) and a C-18 reverse phase column anchored to a reverse phase high-performance liquid chromatography (RP-HPLC) with the same conditions as those described for the natural ones.

### Serum Samples

Individual blood samples from 10 allergic patients with a proven allergy to nsLTPs were collected. The diagnosis of IgE-mediated tomato allergy was made based on a well-defined clinical history of nsLTPs allergy and a positive skin prick test (SPT) to nsLTPs together with evidence of specific IgE antibodies (sIgE). The total nsLTPs sIgE levels in serum samples were determined by ImmunoCAP-FEIA according to manufacturer's instructions (Thermo Fisher Scientific, Uppsala, Sweden).

### IgE Immunoblotting.

CAs were diluted in tricine sample buffer (Bio-Rad, Richmond, CA), with 5% (v/v) of β-mercaptoethanol (β-ME) and were heated at 95° C. for 5 min. Samples were loaded and analyzed on Precast Criterion XT 16.5% Tris-Tricine gels (Bio-Rad). Separations were carried out at 100 V during 1 h and 45 min, using a Tris-Tricine running buffer (Bio-rad) in the Criterion cell.

After SDS-PAGE separation, gels were soaked in transfer buffer (48 mM Tris, 39 mM glycine, 20% methanol, pH 9.2) for 20 min and subjected to semidry transfer in a Trans-Blot SD (Bio-Rad) for 30 min at 18 V. The nitrocellulose membranes were blocked with 3% skim milk powder in phosphate buffered saline, pH 7.4 containing 0.05% Tween 20 PBS-T. The membrane was incubated with a 1:5 dilution of serum form allergic patients. Briefly, the binding of human IgE was detected by using the mouse anti-human IgE antibodies (diluted 1:5000) kindly donated by Alk-Abelló, followed by horseradish peroxidase (HRP)-labeled rabbit anti-mouse IgG (diluted 1:5000; DAKO, Glostrup, Denmark). The signal was developed with a chemiluminescent ECL-Western blotting reagent (GE Healthcare, Chicago, IL).

### Results

CA1 can be recognized by the IgEs present on the samples of 10 different allergic patients, demonstrating that the consensus design conserve the IgE reactive epitopes present in the natural nsLTPs. This demonstrates that the consensus sequences can be used to desensitize allergic patients against natural allergens.

### Example 4

The following may be used to demonstrate how the ability of the CAs to promote class switching from IgE-producing B-cells to IgG producing B-cells on patients sensitized against CAs, specifically CA1-4, can be done.

### Materials and Methods

### Generation of Monocyte-Derived Human DC

Dendritic cells (DCs) could be generated by differentiating monocyte cells (PBMC). PBMC can then be isolated from heparinized blood from healthy donors by Ficoll-Paque 1.077 density centrifugation and subsequently differentiated by incubation with were incubated in 3 ml/well IMDM supplemented with 1% heat-inactivated autologous plasma, 1000 U/ml IL-4 and 200 U/ml GM-CSF. The immature DCs are then to be treated with CAs formulated as a vaccine of the present invention. Ature DC expressing high levels of CD80, CD83, CD86 and MHC class II molecules controlled by flow cytometry (>90%) can be harvested 48 hr after stimulation, washed twice and used for T-cell stimulation assays.

### Purification of T and B Cells.

Autologous CD4+, CD8+ T cells and CD19+ B cells may be obtained from PBMC using antibody-coated paramagnetic MicroBeads according to the protocol of the manufacturer.

### Cytokine Production Assays.

Coculture of T cells and autologous allergen-pulsed or allergen-DNA transfected DC for cytokine production assays, T cells could be co-cultured with primed DCs in 1 ml IMDM supplemented with 5% heat-inactivated autologous plasma On day 7, T cells are to be restimulated with 5×10⁴ newly generated autologous allergen-pulsed or allergen-DNA transfected DC, and supernatants may be collected 24 hr later.

### Immunoglobulin Production Assay.

To measure immunoglobulin production, B cells, T cells, DCs and CD40L transfected L cells will be cocultured in the presence of IL.4. After 12 days supernatants will be collected and amount of total IgE, allergen specific IgE, total and allergen specific IgG4 will be measured by ELISA.

### Quantification of Cytokine and Immunoglobulin Production by ELISA.

Human IL-4, IL-5, IL-10, IFN-γ, total IgE and IgG4 could be measured by ELISA according to the instructions of the distributors of the employed pairs of antibodies.

### Expected Results

The expected outcome would be increased levels of IFN-γ, IL-10 (anti-inflammatory cytokines linked to tolerance) and increased levels of IL-4 and IL-5, since this cytokine is involved in stimulating antibody production.

We expect that the total and allergen specific IgE levels are reduced when the cells are treated with CAs, while the amount of allergen specific IgG4s increases.

### Example 5

In this prophesized example it is demonstrated how CAs could offer a prophylactic broad protection against allergy.

### Materials and Methods

BALB/c mice may be vaccinated with CAs in DNA, mRNA and polypeptide format or vehicle intradermally. Subsequently, animal may be sensitized by means of inhalation of a natural allergen/alum, followed by airways provocation with natural allergens from different sources.

IgG1/IgG2a/IgE titters could be determined by using ELISAs. Measurement of cytokines in splenocyte cultures and bronchoalveolar lavage fluids could be performed by ELISA.

### Expected Results

It is to be expected that vaccination proving its antiallergic efficacy in terms of IG subclass distribution, suppression of allergen specific IgEs, reduction of IL-4 and IL-5 levels, and induction of IFN-γ-producing cells.

### Example 6

In this example it is demonstrated how CAs in polypeptide or nucleotide format could be tested a broad-protective treatment for allergy in an animal allergy model.

### Materials and Methods

Four- to five-week-old BALB/c female mice could be separated in various groups including DNA, mRNA, polypeptide treated and non-sensitized groups. Mice sensitization could be carried out using Pru p 3 (SEQ ID NO: 17, a representative nsLTP), or a similar protein allergen in combination with the adjuvant lipopolysaccharides via intranasal administration. The same schedule will be followed but with PBS (phosphate buffer saline buffer) for the non-sensitized group. Once sensitized, at week 6, mice could be treated by subdermal administration of DNA, mRNA or polypeptide CAs.

To assess anaphylaxis, sensitized mice could be challenge one week after the last sensitization dose with one intraperitoneal dose of Pru p 3 and for example Mal d 3 (another relevant nsLTP, SEQ ID NO: 19). To assess tolerance, mice could be challenge with one intraperitoneal dose of Pru p 3 and for example Mal d 3 either one or three weeks after the last doses of vaccination (week 14 or 16 respectively).

### In Vivo Evaluation of the Response.

The appearance of systemic anaphylaxis could be evaluated at weeks 14 and 16, within approximately 30-40 min after challenge with one intraperitoneal dose of natural allergens by measuring change in body temperature with a rectal probe.

Allergen specific IgE, IgG1, IgG2 in the mice sera could be evaluated using ELISA.

### Expected Results

It is to be expect that the mice treated with CAs are protected from anaphylaxis following natural allergen challenge, in example by not showing change in body temperature. A reduced level of allergen-specific IgE and IgG1, while higher levels of IgG2a compared to the untreated group is an expected outcome.

### Example 7

In this example it is demonstrated how CAs in polypeptide or nucleotide format could be tested a broad-protective treatment for allergy in a naïve animal model.

### Materials and Methods

mRNA-LNPs Preparation

The mRNA encoding CA1 was purchased to RiboPro, including 150 nt Poly-A tail and Cap1. The mRNA was kept at −80 C until encapsulation and fresh LNPs were prepared prior to each injection. The LNP formulation used for the study is based on the Pfizer/BioNTech vaccine Comirnaty (BNT162b2), relying on the ionizable lipid ALC-0315. The PEGylated lipid ALC-0159 lipid is however exchanged with DMG-PEG. The molar ratio of lipid and the lipid-to-mRNA ratio is kept as in BNT162b2. Lipids are dissolved in ethanol. mRNA is dissolved in Sodium Acetate (pH 4.0). Particles are formed by mixing the two phases at a 1:3 ratio using the Ignite microfluidic mixer at a final concentration of 100 μg/mL mRNA. Following mixing, the buffer is exchanged into HBS by discontinuous diafiltration (with spin columns). In the same step, the particles are concentrated to 0.35 μg/μL.

The particles are characterized with DLS and PALS to determine size, polydispersity, and zeta potential. The particles are also characterized by Ribogreen assay, to determine mRNA encapsulation efficiency and final mRNA yield and concentration. The particles are diluted in sucrose to the desired concentrations and volumes and kept at −80 C until use.

### Polypeptide Preparation

CAs cDNA and protein was obtained as described in Example 3.

The purity of the CA tested by diluting the proteins in tricine sample buffer (Bio-Rad, Richmond, CA), with 5% (v/v) of β-mercaptoethanol (β-ME) and were heated at 95° C. for 5 min. Samples were loaded and analyzed on Precast Criterion XT 16.5% Tris-Tricine gels (Bio-Rad). Separations were carried out at 100 V during 1 h and 45 min, using a Tris-Tricine running buffer (Bio-rad) in the Criterion cell. The SDS-PAGE was stained with staining solution (0.1% (w/v) Coomassie Brilliant Blue R-250, 50% ethanol and 10% acetic acid) for 30 min and de-stained with the same solution but without Coomasie Brilliant Blue R-250.

The CA1 was dialyzed against NH₄HCO₅ 50 mM pH 7.0 to equilibrium and then lyophilized. The powder was stored at −20° C. until use.

The protein was dissolved in sterile PBS containing 50 μg/mL of Polyinosinic-polycytidylic acid sodium salt (Poly (Poly (I:C) #P1530, Sigma-Aldrich). The protein solution was filtered through a 0.22 μm filter (Milipore) in sterial conditions prior to injection to the mice. Sterile protein solution was kept for maximum 1 day before administration to mice.

Four- to five-week-old BALB/c female mice were separated in groups or three mice each. Mice were immunised with three different doses of mRNA-LNP (9, 3, 0.6 mg), polypeptide treated (24, 12 or 6 mg). Mice sensitization was carried out using CA1 (SEQ ID NO: 1, a consensus nsLTP), via subdermal administration. The same schedule will be followed but with PBS (phosphate buffer saline buffer) for the non-sensitized group.

Mice are immunised on day 1, day 21, and day 42 by subcutaneous administration of three different doses of, mRNA (9, 3, 0.6 mg) or polypeptide CA1 (24, 12 or 6 mg). On day 63, mice are sacrificed by cardiac puncture.

### Mice Blood Samples

Prior to every immunization (day 0, 21, 42) and before sacrifice of the mice (day 63) blood was extracted from mice and transferred to Eppendorf tubes. After 30 min on ice, the clotted blood was centrifuged at 1000×g for 10 min at 4° C. The supernatant (serum) was transferred to new tubes, aliquoted and frozen at −20° C. until use.

### Human Blood Samples

Individual blood samples from 4 allergic patients with a proven allergy to nsLTPs were collected. The diagnosis of IgE-mediated peach allergy was made based on a well-defined clinical history of nsLTPs allergy and a positive skin prick test (SPT) to nsLTPs together with evidence of specific IgE antibodies (sIgE). The total nsLTPs sIgE levels in serum samples were determined by ImmunoCAP-FELA according to manufacturer's instructions (Thermo Fisher Scientific, Uppsala, Sweden).

### Evaluation of Antigen Specific IgG1, IgG2a, and IgE

Half-area 96-well High binding plates were coated with CA1 polypeptide by adding 50 μL of a 10 μg/mL solution and incubating over night at 4° C. The plates were washed three times with PBS containing 0.1% (v/v) Tween 20 and then three times with PBS. Then the wells were blocked with PBS containing 0.1% (w/v) of Bovine Serum Albumin (#A3294 Sigma-Aldrich) for 2 h at room temperature. The plates were then washed three times with PBS containing 0.1% (v/v) Tween 20 and then three times with PBS. 25 μL of PBS containing 0.05% (w/v) of Bovine Serum Albumin was added to all wells. For the IgG1 and IgG2a titration, serum was diluted 1:500 (mRNA, PBS) or 1:1000 (protein). 25 μL of serum was added to the wells and incubated for 1 h at room temperature. After the incubation time, the plates were washed again as described above and 1:250 diluted anti-mouse specific antibodies supplied in Ig Isotyping Mouse Uncoated ELISA Kit (#88-50630, Invitrogen) were added to wells and incubated for 1 h at room temperature. Plates were washed as described, and 1:500 diluted Goat anti-Rat IgG (H+L) Cross-Adsorbed Secondary Antibody, HRP (#A10549, Invitrogen) was added to wells and incubated for 1 h. The plates were washed as described. For detection of bound antibodies, 50 μL Tetramethylbenzidine (TMB) substrate solution was added to all wells and incubated for 15 min at room temperature. To stop the reaction, 1M H2SO4 was added, and the plate was read at 450 nm.

For determination of allergen specific IgE in mice, ELISA MAX™ Standard Set Mouse IgE (#432401, Biolegend) was used with a modified protocol based on the manufacturer's indications. Half-area 96-well High binding plates were coated with CA1 polypeptide by adding 50 μL of a 10 μg/mL solution and incubated over night at 4° C. After washing and blocking steps, as described above, 50 μL of mouse serum diluted 1:10 was added to the wells and incubated for 2 h. After washing of the plate, 50 μL of 1:200 diluted anti-mouse IgE specific antibody supplied in the kit was added to the wells and incubated for 1 h at room temperature. Plates were washed as described, and 50 μL of 1:200 diluted detection antibody was added to wells and incubated for 1 h at room temperature. The plate was washed, and 50 μL of 1:1000 diluted Avidin-HRP solution was added to wells and incubated for 30 min at room temperature. The plates were washed as described. For detection of bound antibodies, 50 μL Tetramethylbenzidine (TMB) substrate solution was added to all wells and incubated for 15 min at room temperature. To stop the reaction, 1M H2SO4 was added, and the plate was read at 450 nm.

### Evaluation of Inhibition Capacity of Mouse Serum Antibodies for Human IgE

Half-area 96-well High binding plates were coated with CA1 polypeptide by adding 50 μL of a 10 μg/mL solution and incubating over night at 4° C. The plates were washed three times with PBS containing 0.1% (v/v) Tween 20 and then three times with PBS. Then the wells were blocked with PBS containing 0.1% (w/v) of Bovine Serum Albumin (#A3294 Sigma-Aldrich) for 2 h at room temperature. The plates were then washed three times with PBS containing 0.1% (v/v) Tween 20 and then three times with PBS. 25 ul of PBS containing 0.05% (w/v) of Bovine Serum Albumin was added to all wells followed by addition of 25 ul of serum from mice immunized with mRNA-LNP and protein in varying dilutions (1:10, 1:20, 1:100, 1:500, 1:1000, 1:5000, 1:15000). Plates were incubated for 1 h at room temperature. After the incubation time, the plates were washed again as described above. 25 μL of PBS containing 0.05% (w/v) of Bovine Serum Albumin was added to all wells followed by addition of 25 μL 1:10 diluted serum from severely allergic human donors. The plate was incubated for 1 h at room temperature and washed as described. Goat 1:2000 anti-Human IgE Secondary Antibody, HRP (#A18793, Invitrogen) were added to wells and incubated for 1 h at room temperature. The plates were washed and 50 μL Tetramethylbenzidine (TMB) substrate solution was added to all wells and incubated for 15 min at room temperature. To stop the reaction, 1M H₂SO₄ was added, and the plate was read at 450 nm.

### In Vivo Evaluation of the Response

The appearance of secondary effects such as fever or discomfort was followed by observation within approximately 30-40 min after subcutaneous. No secondary effects following injections were observed in any of the mice.

Allergen specific IgE, IgG1, IgG2a in the mice sera was evaluated using ELISA.

The serum samples from mice immunised with the highest dose of mRNA-LNPs and protein were testes for the capacity of blocking the binding of IgEs present in the serum of patients severely allergic to peaches to CA1 or Pru p3 (major nsLTP allergen from peach).

### Results

### Immunization of Naïve Mice

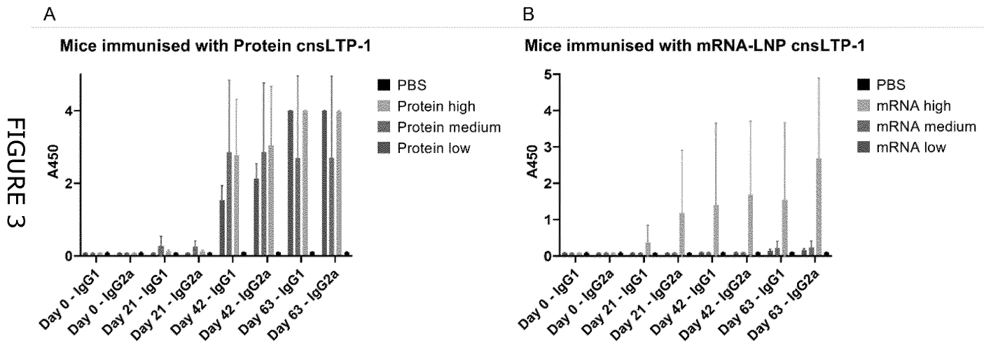

21 days after the first injection of naïve mice with mRNA encoding CA1 or CA1 protein, the mice treated with mRNA-LNP, showed an increase in the CA1 specific IgG1 and IgG2a compared to the untreated group (FIG. 3B). This was not observed for the mice treated with CA1 protein (FIG. 3A). The mice treated with CA1 proteins did not show a response until after the second injection at 42 days, suggesting the need for multiple injections of CA1 protein is required to obtain the desired immunological response, which was to a large extend independent on the protein concentration injected (FIG. 3A).

Upon injection of the subsequent booster doses, the IgG titers increase for both protein and mRNA-LNP treated groups, reaching higher titers in the protein group than in the mRNA-LNP group (FIG. 3).

During the immunization of mice with mRNA encoding CA1 or CA1 protein (FIG. 3) the mice did not develop any visible discomfort or temperature change after injections with any of the CA formats or doses assayed.

The ratio of IgG1/IgG2a may be used as an indicator for the type of immunological response obtained by the immunization. In general, an IgG1/IgG2a ratio >1 indicates a T-helper cell type 2 (Th2) driven humoral immune response, while IgG1/IgG2a ratio <1 ratio tends to indicate a T-helper cell type 1 (Th1) type cellular immune response. In the context of vaccine development and allergy, the Th2 response is related to allergic responses, where allergen exposure, such as exposure to pollen or dust mites, stimulates the Th2 immune response, which release cytokines that promote the production of IgE antibodies, which bind to mast cells and basophils. Upon subsequent exposure to the same allergen, these IgE-coated cells release inflammatory mediators like histamine, leading to allergic symptoms such as itching, swelling, and respiratory distress. Accordingly, an elevated IgG1/IgG2a ratio i.e., >1 is often associated with an elevated IgE response.

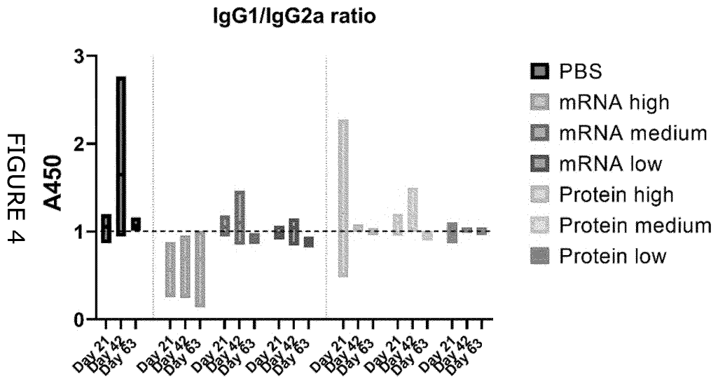

For all the immunization conditions the IgG1/IgG2a ratio was calculated (FIG. 4), and it was found that for the high dose of mRNA-CA1, the IgG1/IgG2a ratio was <1 indicating a Th1 response in the mRNA treated group, while the protein-based immunization with CA1 resulted in a IgG1/IgG2a ratio of about 1 (FIG. 4), suggesting an inconclusive Th1/Th2 balance response.

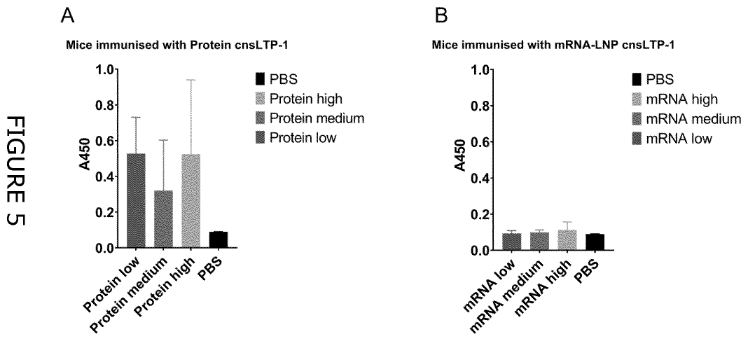

This tendency was also validated when measuring the levels of the allergen-specific (CA1, cnsLTP-1 in FIG. 5) IgEs, where mice immunised with the CA1 protein showed an induced production of allergen-specific IgEs, while the mRNA-LNP immunised group do not show the production of IgEs (FIG. 5).

Accordingly, this shows that the mRNA based CA1 allergy vaccine promotes a beneficial immunological response in naïve mice, without inducing production of high levels of IgE antibodies.

### Human Antibody Recognition

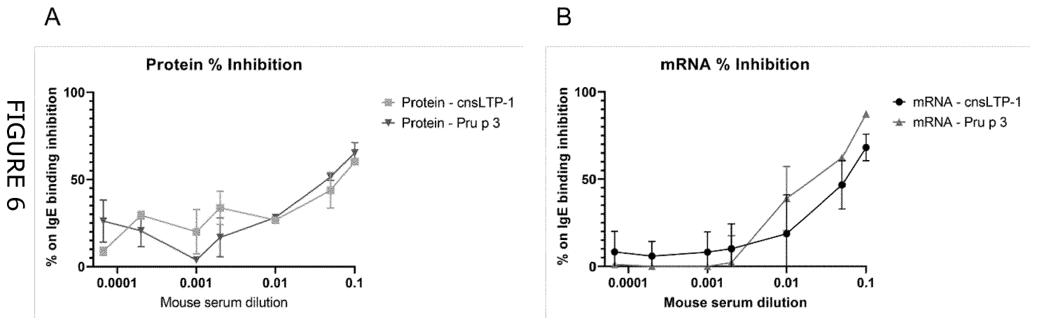

To test if the antibodies produced by the immunised mice could block the binding of human IgEs from allergic patients to CA1 (cnsLTP-1 in FIG. 6), a dose response blocking experiments was conducted, with immobilized CA1, where the mouse IgE's were used to block the binding of human IgE's obtained from severely allergic patients.

As is shown in FIG. 6, the antibodies produced by the mice immunised with mRNA-LNPs inhibit up to 87% of the IgE binding while the immunisation with proteins reaches a max 70% inhibition.

In addition, it was also tested if the mouse produced IgE were able to block the binding of the wild-type allergen Pru P 3, i.e., if the antibodies produced against the CA1 protein were able to recognize the natural allergen Pru P 3, and effectively block the binding of human IgE antibodies.

As is shown in FIG. 6, the antibodies produced via immunization with LNP-mRNA-CA1 and protein CA1 recognised the wild-type allergen Pru P 3 and was able to block the binding of human IgE, strongly suggesting an epitope overlap between the mouse and human antibody.

In summary, the immunization of naïve mice produced CA1 specific antibodies which were able to recognise the wild-type allergen Pru P 3, and the produced antibodies were able to block the recognition of human antibodies to both CA1 and to the natural allergen Pru P 3, suggesting that the consensus allergen-based approach described herein may be used to generate allergy vaccines which are efficacious towards multiple sequence related allergens.

### Example 8

In this experiment, we demonstrate that the designed consensus allergens (CAs) conserve epitopes that can be recognized by IgGs raised against the Bet V1 allergen from Betula pendula, confirming that the consensus allergens can be recognised by epitope specific antibodies.

### Materials and Methods

CAs cDNA sequences optimized for E. coli expression (GeneScript) were purchased (Eurofins genomics) and subcloned into the pET His6 MBP TEV LIC cloning vector in phase with Maltose Binding Protein. The vector was transformed into E. coli BL21DE3 cells and colonies harbouring the plasmid were selected by Kanamycin resistance. The protein was expressed by culturing E. coli in autoinduction media in the presence of Kanamycin for 24 h. The cells were harvested, lysed by sonication, and the soluble cellular fraction was collected by centrifugation. The CAs were purified with an Cytiva 5 mL MBP-Trap, samples with a positive signal in an SDS-PAGE were pooled and treated with TEV protease to eliminate the MBP tag. Finally, the CAs were separated from TEV protease and MBP via size-exclusion chromatograph. CAs were freeze-dried and stored at −20 until use.

CAs were resuspended in PBS and prepared for SDS-PAGE with a reducing loading buffer. 5 μg of proteins were loaded on a 4-12% Bis-Tris polyacrylamide gel. The SDS-PAGE was run according to the manufacturer conditions. The proteins were transferred to a PVDF membrane, blocked with Tris buffered Salt buffer (TBS) containing 0.1% (v/v) of Tween 20 (TBST) and 5% of skimmed milk (TBST-M). The membrane was washed with TBST and incubated with the primary antibody in TBST 0.1% Milk 5% (Ab00648-23.0 anti bet v 1 1/1000). After 1 h incubation, the membrane was washed and incubated with HRP-conjugated diluted in TBST 0.1% Milk 5% (Cayman Goat-anti-Rabbit 1/4000) for 1 h. The result was revealed with and Chemiluminescent substrate.

### Results

As shown in FIG. 2, CA5 (PR10-3), CA6 (PR10-2) and CA8 (PR10-1) can all be recognized by the IgGs specific towards the wild-type bet vi allergen (PR10), demonstrating that the consensus design conserve the IgG recognised epitopes present in the natural PR10 allergens.

1. An allergy vaccine comprising a consensus allergen or a nucleic acid sequence encoding said consensus allergen, wherein the consensus allergen comprises at least 60 amino acids and is derived from a consensus sequence of the amino acid sequences of at least five (5) protein allergens, and wherein said protein allergens share at least 20% amino acid sequence identity.

2-29. (canceled)

30. The allergy vaccine according to claim 1, wherein the consensus allergen is not a wild-type protein allergen.

31. The allergy vaccine according to claim 1, wherein the allergens share at least 20%, 30%, 40%, 50%, 60%, 70%, or at least 80% amino acid sequence identity over a sequence length of at least 60, 65, 70, 85, 90, 95, 100, 110, or 115 amino acids.

32. A method of making the allergy vaccine of claim 1 comprising:
a) selecting at least five (5) amino acid sequences of allergens as defined in claim 1,
b) performing an alignment of the amino acid sequences of said allergens, and
c) determining the consensus sequence of said allergens from said alignment, wherein the selection of amino acids in the consensus sequence is based on the incidence number of the specific amino acid in each specific position (n) in the sequence of said alignment.

33. The method according to claim 32, wherein in said selection of conserved amino acids in the consensus sequence, when two or more amino acids are equally represented in a position (n) in the aligned sequence, the amino acid with the highest molecular volume, according to table 3, is selected as the conserved amino acid, and optionally wherein further when two amino acids remain equally represented in a position (n) following selection of the amino acid with the highest molecular volume, the conserved amino acid is selected based on the physicochemical properties of amino acid according to the following groups:
group 1 [polar] comprising Asn, Gln, Ser, and Thr,
group 2 [aliphatic] comprising Val, Ala, Leu, Ile, and Met,
group 3 [basic] comprising Lys, Arg, and His,
group 4 [acidic] comprising Asp and Glu,
group 5 [aromatic] comprising Phe, Trp, and Tyr,
group 6 comprising Pro,
group 7 comprising Gly, and
group 8 comprising Cys, and
wherein the conserved amino acid in the specific position is selected based on the selection criteria defined in table 2.

34. The allergy vaccine according to claim 1, wherein the vaccine is hypoallergenic.

35. The allergy vaccine according to claim 1, wherein the vaccine comprises a nucleotide construct encoding the consensus allergen.

36. The allergy vaccine according to claim 35, wherein the nucleic acid construct comprises at least one further nucleic acid sequence encoding a polypeptide.

37. The allergy vaccine according to claim 35, wherein the vaccine is an mRNA vaccine.

38. The allergy vaccine according to claim 1, wherein the vaccine comprises a polypeptide comprising the consensus allergen.

39. The allergy vaccine according to claim 1, wherein the vaccine comprises at least one or more adjuvants.

40. The allergy vaccine according to claim 1, wherein said consensus allergen is derived from the consensus sequence of non-specific Lipid Transfer Proteins (nsLTP).

41. The allergy vaccine according to claim 1, wherein said consensus allergen is derived from the consensus sequence of Pathogenic Related proteins family 10 (PR-10) proteins.

42. The allergy vaccine according to claim 1, wherein the consensus allergen comprises an amino acid sequence selected from the group consisting of SEQ ID NOs 1, 2, 3, 4, 20, 21, 22 and 23 and functional homologues thereof with an amino acid sequence which is at least 80%, 85%, 90%, 95%, 97%, 98%, or 99% identical to any one of SEQ ID NOs 1, 2, 3, 4, 20, 21, 22 or 23.

43. The allergy vaccine according to claim 1, wherein the vaccine is formulated for intramuscular, intradermal, intravenous, transdermal, topical, sublingual, subcutaneous, oral, nasal, ophthalmic, or biolistic administration.

44. The allergy vaccine according to claim 1, wherein the vaccine is administered in a dose in the range of 1-1000 ug pr. dose.

45. The allergy vaccine according to claim 37, wherein the vaccine is administered in a dose in the range of 5-50 ug pr. dose.

46. The allergy vaccine according to claim 38, wherein the vaccine is administered in a dose in the range of 10-100 ug pr. dose.

47. The allergy vaccine according to claim 1, wherein the vaccine is administered once or repeatedly.

48. The allergy vaccine according to claim 1, wherein the vaccine is administered by subcutaneous administration.

49. A method of using the allergy vaccine of claim 1 for producing an immunogenic response in a subject comprising administering the allergy vaccine of claim 1 to a subject.

50. The method according to claim 49, wherein the vaccine is administered to said subject as a prophylactic therapy.

51. The method according to claim 49, wherein the vaccine is administered to said subject as an allergy therapy.

52. The method according to claim 51, wherein the subject has a peach-cypress allergy or birch-apple syndrome.

53. The method of claim 49, wherein the vaccine is administered to said subject to ameliorate allergy symptoms.

54. A method for providing a consensus allergen for use in a vaccine, the method comprising:
a) selecting at least five (5) amino acid sequences of allergens as defined in claim 2,
b) performing an alignment of the amino acid sequences of said allergens, and
c) determining the consensus sequence of said allergens from said alignment, wherein the selection of amino acids in the consensus sequence is based on the incidence number of the specific amino acid in each specific position (n) in the sequence of said alignment.

55. The method for providing a consensus allergen for use in a vaccine according to claim 54, wherein in said selection of conserved amino acids in the consensus sequence, when two or more amino acids are equally represented in a position (n) in the aligned sequence, the amino acid with the highest molecular volume, according to table 3, is selected as the conserved amino acid, and optionally wherein further when two amino acids remain equally represented in a position (n) following selection of the amino acid with the highest molecular volume, the conserved amino acid is selected based on the physicochemical properties of amino acid according to the following groups:
group 1 [polar] comprising Asn, Gln, Ser, and Thr,
group 2 [aliphatic] comprising Val, Ala, Leu, Ile, and Met,
group 3 [basic] comprising Lys, Arg, and His,
group 4 [acidic] comprising Asp and Glu,
group 5 [aromatic] comprising Phe, Trp, and Tyr,
group 6 comprising Pro,
group 7 comprising Gly, and
group 8 comprising Cys, and
wherein the conserved amino acid in the specific position is selected based on the selection criteria defined in table 2.

56. An isolated polypeptide comprising a consensus allergen, wherein said consensus allergen is prepared by the method of claim 54.

57. A nucleotide vaccine comprising a nucleic acid construct encoding a consensus allergen, wherein said consensus allergen is prepared by the method of claim 54.
